SEQUENCE LISTING (1010 sequences)

SEQ ID NO: 1-505 - skipped (505 entries)

SEQ ID NO: 506 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507-515 - skipped (9 entries)

SEQ ID NO: 516 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517-519 - skipped (3 entries)

SEQ ID NO: 520 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521-605 - skipped (85 entries)

SEQ ID NO: 606 - protein, 120 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 607-615 - skipped (9 entries)

SEQ ID NO: 616 - protein, 107 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 617-619 - skipped (3 entries)

SEQ ID NO: 620 - protein, 120 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 621-623 - skipped (3 entries)

SEQ ID NO: 624 - protein, 107 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 625-705 - skipped (81 entries)

SEQ ID NO: 706 - protein, 125 aa, Artificial Sequence
  source: /note=“Description of Artificial Sequence: Synthetic polypeptide”
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 707-717 - skipped (11 entries)

SEQ ID NO: 718 - protein, 107 aa, Artificial Sequence
  source: /note=“Description of Artificial Sequence: Synthetic polypeptide”
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 719-723 - skipped (5 entries)

SEQ ID NO: 724 - protein, 125 aa, Artificial Sequence
  source: /note=“Description of Artificial Sequence: Synthetic polypeptide”
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 725-729 - skipped (5 entries)

SEQ ID NO: 730 - protein, 107 aa, Artificial Sequence
  source: /note=“Description of Artificial Sequence: Synthetic polypeptide”
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 731-761 - skipped (31 entries)

SEQ ID NO: 762 - protein, 447 aa, Artificial Sequence
  source: /note=“Description of Artificial Sequence: Synthetic polypeptide”
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 763 - protein, 214 aa, Artificial Sequence
  source: /note=“Description of Artificial Sequence: Synthetic polypeptide”
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 446 aa, Artificial Sequence
  source: /note=“Description of Artificial Sequence: Synthetic polypeptide”
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 220 aa, Artificial Sequence
  source: /note=“Description of Artificial Sequence: Synthetic polypeptide”
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766-805 - skipped (40 entries)

SEQ ID NO: 806 - protein, 118 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807-815 - skipped (9 entries)

SEQ ID NO: 816 - protein, 111 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817-821 - skipped (5 entries)

SEQ ID NO: 822 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823-825 - skipped (3 entries)

SEQ ID NO: 826 - protein, 111 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827-829 - skipped (3 entries)

SEQ ID NO: 830 - protein, 114 aa, Artificial Sequence
  source: /note=“Description of Artificial Sequence: Synthetic polypeptide”
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, Artificial Sequence
  source: /note=“Description of Artificial Sequence: Synthetic polypeptide”
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, Artificial Sequence
  source: /note=“Description of Artificial Sequence: Synthetic polypeptide”
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, Artificial Sequence
  source: /note=“Description of Artificial Sequence: Synthetic polypeptide”
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834-900 - skipped (67 entries)

SEQ ID NO: 901 - protein, 121 aa, Artificial Sequence
  source: /note=“Description of Artificial Sequence: Synthetic polypeptide”
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 902 - protein, 107 aa, Artificial Sequence
  source: /note=“Description of Artificial Sequence: Synthetic polypeptide”
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 903-919 - skipped (17 entries)

SEQ ID NO: 920 - protein, 124 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 921 - protein, 107 aa, Artificial Sequence
  source: /note=“Description of Artificial Sequence: Synthetic polypeptide”
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 922-1000 - skipped (79 entries)

SEQ ID NO: 1001 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1002 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 1003 - protein, 114 aa, Artificial Sequence
  source: /note=“Description of Artificial Sequence: Synthetic polypeptide”
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 1004 - protein, 297 aa, Artificial Sequence
  source: /note=“Description of Artificial Sequence: Synthetic polypeptide”
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1005 - protein, 114 aa, Artificial Sequence; identical to SEQ ID NO: 1001
  source: /note=“Description of Artificial Sequence: Synthetic polypeptide”
  VARIANT (93)..(93): /replace=“Lys”
  MISC_FEATURE (1)..(114): /note=“Variant residues given in the sequence have no preference with respect to those in the annotations for variant positions”

SEQ ID NO: 1006 - protein, 77 aa, Artificial Sequence
  source: /note=“Description of Artificial Sequence: Synthetic polypeptide”
ITCPPPMSVEHADIWVKSYSLYSRERYICNSGFKRKAGTSSLTECVLNKATNVAHWTTPSLKCIRDPALVHQRPAPP

SEQ ID NO: 1007 - protein, 214 aa, Artificial Sequence
  source: /note=“Description of Artificial Sequence: Synthetic polypeptide”
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1008 - protein, 448 aa, Artificial Sequence
  source: /note=“Description of Artificial Sequence: Synthetic polypeptide”
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1009 - protein, 445 aa, Artificial Sequence
  source: /note=“Description of Artificial Sequence: Synthetic polypeptide”
QVQLQESGPGLVKPSQTLSLTCSFSGFSLSTSGMGVGWIRQPSGKGLEWLAHIWWDGDESYNPSLKSRLTISKDTSKNQVSLKITSVTAADTAVYFCARNRYDPPWFVDWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 1010 - protein, 214 aa, Artificial Sequence
  source: /note=“Description of Artificial Sequence: Synthetic polypeptide”
DIQMTQSTSSLSASVGDRVTITCRASQDISNYLSWYQQKPGKAVKLLIYYTSKLHSGVPSRFSGSGSGTDYTLTISSLQQEDFATYFCLQGKMLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0056123 A1
(43) Pub. Date: Feb. 24, 2022

(54) USE OF IL-1BETA BINDING ANTIBODIES

(71) Applicant: NOVARTIS AG, Basel (CH)

(72) Inventors: Mark SHILKRUT, Hoboken, NJ (US); Michelle TSAI, Basel (CH); Connie WONG, Arlington, MA (US)

(21) Appl. No.: 17/415,298

(22) PCT Filed: Dec. 18, 2019
(86) PCT No.: PCT/IB2019/001347

§ 371 Date: Jun. 17, 2021

Related U.S. Application Data
(60) Provisional application No. 62/783,642, filed on Dec. 21, 2018.
(60) Provisional application No. 62/916,497, filed on Oct. 17, 2019.

(51) Int. Cl. C07K 16/24; A61K 45/06; A61P 35/04

(52) U.S. Cl. CPC C07K 16/245; A61K 2039/505; A61P 35/04; A61K 45/06

(57) ABSTRACT

Use of an IL-1β binding antibody or a functional fragment thereof, especially canakinumab or a functional fragment thereof, or gevokizumab or a functional fragment thereof, and biomarkers for the treatment and/or prevention of a cancer having at least a partial inflammatory basis.

### TECHNICAL FIELD

The present invention relates to the use of an IL-1β binding antibody or a functional fragment thereof, for the treatment and/or prevention of cancers, e.g., cancers having at least a partial inflammatory basis.

### BACKGROUND OF THE DISCLOSURE

The majority of cancers is still incurable. There remains a continued need to develop new treatment options for cancers.

### SUMMARY OF THE DISCLOSURE

The present invention/disclosure relates to the use of an IL-1β binding antibody or a functional fragment thereof, suitably canakinumab, suitably gevokizumab, for the treatment and/or prevention of cancers, e.g., cancers that have at least a partial inflammatory basis. Typically cancers, e.g., cancers that have at least a partial inflammatory basis include lung cancer, especially NSCLC, colorectal cancer (CRC), melanoma, gastric cancer (including esophageal cancer), renal cell carcinoma (RCC), breast cancer, prostate cancer, head and neck cancer (including oral), bladder cancer, hepatocellular carcinoma (HCC), ovarian cancer, cervical cancer, endometrial cancer, pancreatic cancer, neuroendocrine cancer, hematological cancer (particularly multiple myeloma, acute myeloblastic leukemia (AML)), and biliary tract cancer.

In another aspect, the present invention/disclosure relates to a particular clinical dosage regimen for the administration of an IL-1β binding antibody or a functional fragment thereof, suitably canakinumab, suitably gevokizumab, for the treatment and/or prevention of cancer, e.g., cancers having at least a partial inflammatory basis. In one embodiment, the preferred dose of canakinumab for a patient with cancer that has at least a partial inflammatory basis is about 200 mg every 3 weeks or monthly, preferably subcutaneously. In one embodiment, patient receives gevokizumab about 30 mg to about 120 mg per treatment every 3 weeks or monthly, preferably intravenously.

In another aspect, the subject with cancer, e.g., cancer having at least a partial inflammatory basis, is administered with one or more anti-cancer therapeutic agent (e.g., a chemotherapeutic agent) and/or have received/will receive debulking procedures in addition to the administration of an IL-1β binding antibody or a functional fragment thereof, suitably canakinumab, suitably gevokizumab.

There are also provided methods of treating or preventing cancers, e.g., cancers having at least a partial inflammatory basis in a human subject comprising administering to the subject a therapeutically effective amount of an IL-1β binding antibody or a functional fragment thereof.

The term “therapeutically effective amount” refers to an amount of a drug that will elicit the desired biological and/or medical response of a tissue, system or an animal (including man) that is being sought by a researcher or a clinician. Suitably the term “therapeutically effective amount” refers to an amount of a drug that will elicit the desired biological and/or medical response in a patient in need thereof or in a subject in need thereof, that is being sought by a researcher or a clinician. Another aspect of the invention/disclosure is the use of an IL-1β binding antibody or a functional fragment thereof for the preparation of a medicament for the treatment of cancers, e.g., cancers having at least a partial inflammatory basis.

The present invention/disclosure also provides a pharmaceutical composition comprising a therapeutically effective amount of an IL-1β binding antibody or a functional fragment thereof, suitably canakinumab or gevokizumab, for use in the treatment and/or prevention of cancers, e.g., cancers having at least a partial inflammatory basis. In one embodiment, the pharmaceutical composition comprising a therapeutically effective amount of an IL-1β binding antibody or a functional fragment thereof, e.g., canakinumab, e.g., gevokizumab, is loaded in an auto-injector. In one embodiment, about 200 mg of canakinumab is loaded in an auto-injector. In one embodiment, about 250 mg of canakinumab is loaded in an auto-injector.

The present invention also relates to high sensitivity C-reactive protein (hsCRP) for use as a biomarker in the diagnosis, patient selection, and/or prognosis of cancer treatment, e.g., cancer having at least a partial inflammatory basis. The present invention also relates to high sensitivity C-reactive protein (hsCRP) for use as a biomarker in treatment and/or prevention of cancer having at least a partial inflammatory basis. In a further aspect, the invention relates to high sensitivity C-reactive protein (hsCRP) for use as a biomarker in the treatment and/or prevention of cancer having at least a partial inflammatory basis in a patient, wherein said patient is treated with an IL-1β inhibitor, an IL-1β binding antibody or a functional fragment thereof (e.g., canakinumab or gevokizumab). In one aspect, the patient has hsCRP equal to or greater than about 2.2 mg/L, equal to or greater than about 4.2 mg/L, equal to or greater than about 6.2 mg/L, or equal to or greater than about 10.2 mg/L, before first administration of an IL-1β inhibitor, e.g., an IL-1β binding antibody or functional fragment thereof (e.g., canakinumab or gevokizumab).

In one aspect, the present invention provides an IL-1β binding antibody or a functional fragment thereof (e.g., canakinumab or gevokizumab), for use in a patient in the treatment and/or prevention of a cancer, e.g., a cancer having at least partial inflammatory basis, e.g., a cancer described herein but excluding lung cancer, especially excluding NSCLC. Furthermore, a cancer described herein, but excludes breast cancer. Furthermore, a cancer described herein, but excludes CRC. Each and every embodiments disclosed in this application applies, separately or in combination, to this aspect.

In one aspect the present invention provides an IL-1β binding antibody or a functional fragment thereof (e.g., canakinumab or gevokizumab), for use in a patient in need thereof in the treatment and/or prevention of a cancer selected from a list consisting of lung cancer, especially NSCLC, colorectal cancer (CRC), melanoma, gastric cancer (including esophageal cancer), renal cell carcinoma (RCC), breast cancer, prostate cancer, head and neck cancer (including oral), bladder cancer, hepatocellular carcinoma (HCC), ovarian cancer, cervical cancer, endometrial cancer, pancreatic cancer, neuroendocrine cancer, hematological cancer (particularly multiple myeloma, acute myeloblastic leukemia (AML)), and biliary tract cancer.

### FIGURE LEGENDS

FIG. 1. In vivo model of spontaneous human breast cancer metastasis to human bone predicts a key role for IL-1β signaling in breast cancer bone metastasis. Two 0.5 cm³ pieces of human femoral bone were implanted subcutaneously into 8-week old female NOD SCID mice (n=10/group). 4 weeks later luciferase labelled MDA-MB-231-luc2-TdTomato or T47D cells were injected into the hind mammary fat pads. Each experiment was carried out 3-separate times using bone form a different patient for each repeat. Histograms showing fold change of IL-1B, IL-1R1, Caspase 1 and IL-1Ra copy number (dCT) compared with GAPDH in tumour cells grown in vivo compared with those grown in a tissue culture flask (a i); mammary tumours that metastasise compared with mammary tumours that do not metastasise (a ii); circulating tumour cells compared with tumour cells that remain in the fat pad (a iii) and bone metastases compared with the matched primary tumour (a iv). Fold change in IL-1β protein expression is shown in (b) and fold change in copy number of genes associated with EMT (E-cadherin, N-cadherin and JUP) compared with GAPDH are shown in (c). *=P<0.01**=P<0.001, ***=P<0.0001, \hat{ }\hat{ }\hat{ }=P<0.001 compared with naïve bone.

FIG. 2. Stable transfection of breast cancer cells with IL-1B. MDA-MB-231, MCF7 and T47D breast cancer cells were stably transfected with IL-1B using a human cDNA ORF plasmid with a C-terminal GFP tag or control plasmid. a) shows pg/ng IL-1β protein from IL-1β-positive tumour cell lysates compared with scramble sequence control. b) shows pg/ml of secreted IL-1β from 10,000 IL-1β+ and control cells as measured by ELISA. Effects of IL-1B overexpression on proliferation of MDA-MB-231 and MCF7 cells are shown in (c and d) respectively. Data shown are mean+/−SEM, *=P<0.01, **=P<0.001, ***=P<0.0001 compared with scramble sequence control.

FIG. 3. Tumour derived IL-1β induces epithelial to mesenchymal transition in vitro. MDA-MB-231, MCF7 and T47D cells were stably transfected with to express high levels of IL-1B, or scramble sequence (control) to assess effects of endogenous IL-1B on parameters associated with metastasis. Increased endogenous IL-1B resulted tumour cells changing from an epithelial to mesenchymal phenotype (a). b) shows fold-change in copy number and protein expression of IL-1B, IL-1R1, E-cadherin, N-cadherin and JUP compared with GAPDH and β-catenin respectively. Ability of tumour cells to invade towards osteoblasts through Matrigel and/or 8 μM pores, are shown in (c) and capacity of cells to migrate over 24 and 48 h is shown using a wound closure assay (d). Data are shown as mean+/−SEM, *=P<0.01, **=P<0.001, ***=P<0.0001.

FIG. 4. Pharmacological blockade of IL-1B inhibits spontaneous metastasis to human bone in vivo. Female NOD-SCID mice bearing two 0.5 cm³ pieces of human femoral bone received intra-mammary injections of MDA-MB-231Luc2-TdTomato cells. One week after tumour cell injection mice were treated with 1 mg/kg/day IL-1Ra, 20 mg/kg/14-days canakinumab, or placebo (control) (n=10/group). All animals were culled 35 days following tumour cell injection. Effects on bone metastases (a) were assessed in vivo and immediately post-mortem by luciferase imaging and confirmed ex vivo on histological sections. Data are shown as numbers of photons per second emitted 2 minutes following sub-cutaneous injection of D-luciferin. Effects on numbers of tumour cells detected in the circulation are shown in (b). *=P<0.01. **=P<0.001. ***=P<0.0001.

FIG. 5. Tumour derived IL-1B promotes breast cancer bone homing in vivo. 8-week old female BALB/c nude mice were injected with control (scramble sequence) or IL-1B overexpressing MDA-MB-231-IL-1B+ cells via the lateral tail vein. Tumour growth in bone and lung were measured in vivo by GFP imaging and findings confirmed ex vivo on histological sections. a) shows tumour growth in bone; b) shows representative pCT images of tumour bearing tibiae and the graph shows bone volume (BV)/tissue volume (TV) ratio indicating effects on tumour induced bone destruction; c) shows numbers and size of tumours detected in lungs from each of the cell lines. *=P<0.01, **=P<0.001, ***=P<0.0001. (B=bone, T=tumour, L=lung) FIG. 6. Tumour cell-bone cell interactions stimulate IL-1B production cell proliferation. MDA-MB-231 or T47D human breast cancer cell lines were cultured alone or in combination with live human bone, HS5 bone marrow cells or OB1 primary osteoblasts. a) shows the effects of culturing MDA-MB-231 or T47D cells in live human bone discs on IL-1β concentrations secreted into the media. The effect of co-culturing MDA-MB-231 or T47D cells with HS5 bone cells on IL-1β derived from the individual cell types following cell sorting and the proliferation of these cells are shown in b) and c). Effects of co-culturing MDA-MB-231 or T47D cells with OB1 (osteoblast) cells on proliferation are shown in d). Data are shown as mean+/−SEM, *=P<0.01, **=P<0.001, ***=P<0.0001.

FIG. 7. IL-1β in the bone microenvironment stimulates expansion of the bone metastatic niche. Effects of adding 40 pg/ml or 5 ng/ml recombinant IL-1β to MDA-MB-231 or T47D breast cancer cells is shown in (a) and effects on adding 20 pg/ml, 40 pg/ml or 5 ng/ml IL-1B on proliferation of HS5, bone marrow, or OB1, osteoblasts, are shown in b) and c) respectively. (d) IL-1 driven alterations to the bone vasculature was measured following CD34 staining in the trabecular region of the tibiae from 10-12-week old female IL-1R1 knockout mice. (e) BALB/c nude mice treated with 1 mg/ml/day IL-1Ra for 31 days and (f) C57BL/6 mice treated with 10 μM canakinumab for 4-96 h. Data are shown as mean+/−SEM, *=P<0.01, **=P<0.001, ***=P<0.0001.

FIG. 8. Suppression of IL-1β signalling affects bone integrity and vasculature. Tibiae and serum from mice that do not express IL-1R1 (IL-1R1 KO), BALB/c nude mice treated daily with 1 mg/kg per day of IL-1R antagonist for 21 and 31 days and C57BL/6 mice treated with 10 mg/kg of canakinumab (Ilaris) of 0-96 h were analysed for bone integrity by pCT and vasculature using ELISA for Endothelin 1 and pan VEGF. a) shows the effects of IL-1R1 KO; b) effects of Anakinra and c) effects of canakinumab on bone volume compared with tissue volume (i), concentration of Endothelin 1 (ii) and concentrations of VEGF secreted into the serum. Data shown are mean+/−SEM, *=P<0.01, **=P<0.001, ***=P<0.0001 compared with control.

FIG. 9. Tumour derived IL-1β predicts future recurrence and bone relapse in patients with stage II and III breast cancer. ˜1300 primary breast cancer samples from patients with stage II and III breast cancer with no evidence of metastasis were stained for 17 kD active IL-1β. Tumours were scored for IL-1β in the tumour cell population. Data shown are Kaplan Meyer curves representing the correlation between tumour derived IL-1β and subsequent recurrence a) at any site or b) in bone over a 10-year time period.

FIG. 10. Simulation of canakinumab PK profile and hsCRP profile. a) shows canakinumab concentration time profiles. Solid line and band: median of individual simulated concentrations with 2.5-97.5% prediction interval (300 mg Q12W (bottom line), 200 mg Q3W (middle line), and 300 mg Q4W (top line). b) shows the proportion of month 3 hsCRP being below the cut point of 1.8 mg/L for three different populations: all CANTOS patients (scenario 1), confirmed lung cancer patients (scenario 2), and advanced lung cancer patients (scenario 3) and three different dose regimens. c) is similar to b) with the cut point being 2 mg/L. d) shows the median hsCRP concentration over time for three different doses. e) shows the percent reduction from baseline hsCRP after a single dose.

FIG. 11. Gene expression analysis by RNA sequencing in colorectal cancer patients receiving PDR001 in combination with canakinumab, PDR001 in combination with everolimus and PDR001 in combination with others. In the heatmap figure, each row represents the RNA levels for the labelled gene. Patient samples are delineated by the vertical lines, with the screening (pre-treatment) sample in the left column, and the cycle 3 (on-treatment) sample in the right column. The RNA levels are row-standardized for each gene, with black denoting samples with higher RNA levels and white denoting samples with lower RNA levels. Neutrophil-specific genes FCGR3B, CXCR2, FFAR2, OSM, and G0S2 are boxed.

FIG. 12. Clinical data after gevokizumab treatment (panel a) and its extrapolation to higher doses (panels b, c, and d). Adjusted percent change from baseline in hsCRP in patients in a). The hsCRP exposure-response relationship is shown in b) for six different hsCRP base line concentrations. The simulation of two different doses of gevokizumab is shown in b) and c).

FIG. 13. Effect of anti-IL-1β treatment in two mouse models of cancer. a), b), and c) show data from the MC38 mouse model, and d) and e) show data from the LL2 mouse model.

FIG. 14. Efficacy of canakinumab in combination with pembrolizumab in inhibiting tumor growth.

FIG. 15. Preclinical data on the efficacy of canakinumab in combination with docetaxel in the treatment of cancer.

FIG. 16. Mice were implanted with 4T1 cells sc and treated with the indicated treatments on days 8 and 15 post tumor implant. There were 10 mice in each group.

FIG. 17. Neutrophils (top) and monocytes (bottom) in 4T1 tumors 5 days after a single dose of docetaxel, 01BSUR, or the combination of docetaxel and 01BSUR.

FIG. 18. Granulocytic (top) and monocytic (bottom) MDSC in 4T1 tumors 5 days after a single dose of docetaxel, 01BSUR, or the combination of docetaxel and 01BSUR.

FIG. 19. TIM-3+ CD4⁺ (top) and CD8⁺ (bottom) T cells in 4T1 tumors 4 days after a second dose of docetaxel, 01BSUR, or the combination of docetaxel and 01BSUR.

FIG. 20. TIM-3 expressing Tregs in 4T1 tumors 4 days after a second dose of docetaxel, 01BSUR, or the combination of docetaxel and 01BSUR.

FIG. 21 (a) IL-1β blockade results in delayed tumor growth in NSCLC, TNBC and CRC humanized BLT models. (b) Canakinumab demonstrates immunomodulatory effects including an increase in CD8 TILs in NSCLC H358 model. (c) Gevokizumab/anti-VEGF combination modulates peripheral myeloid populations, including a decrease in tolerogenic DC-10 population in CRC CW480 model.

FIG. 22 (a) Anti-IL-1β modulates myeloid and T cell responses in 4T1 model of TNBC. (b) Docetaxel/anti-IL-1β combo slows tumor growth vs monotherapies and decreases immunosuppressive myeloid cells.

FIG. 23 (a) Tumor volume reduction seen in the combination arm with anti-VEGF is driven by anti-VEGF (b) IL-1β/VEGF blockade remodels the TME differentially as a combination or as single agents (c) IL-1β blockade downregulates FoxP3+ Tregs and improves Teff responses within the tumor.

FIG. 24. A. Schematic of anti-IL-1β and anti-PD-1 antibody treatment regimen. Treatment was initiated one week post orthotopic implantation of KPC cells. Green arrows indicate anti-PD-1 antibody administration while red arrows correspond to anti-IL-1β antibody treatment. B. Graph represents quantification of analysis in A, indicating tumor weight (N=8). Error bars indicate SD; P-values determined by the Student t test (two-tailed, unpaired). Data representative of 2 independent experiments. C. Representative flow cytometry plots (left) of KPC tumors treated with vehicle control, anti-PD-1 antibody alone, anti-IL-1β antibody alone or bothanti-PD-1 and anti-IL-1β antibody, indicating tumor infiltrating CD8⁺ T cells. Graphs depict quantitation of FACS analysis, represented as either percentage of CD45⁺ immune cells (top right, N=8) or absolute number of CD8⁺ T cells relative to tumor weight (bottom right, N=7). Error bars indicate SD; P-values determined by the Student t test (two-tailed, unpaired). Data representative of 2 independent experiments. *p<0.05; **p<0.01; ***p<0.001; ****p<0.0001.

### DETAILED DESCRIPTION OF THE DISCLOSURE

Many malignancies arise in areas of chronic inflammation (1) and inadequate resolution of inflammation is hypothesized to play a major role in tumor invasion, progression, and metastases (Voronov E, et al, PNAS 2003).

As reported in Rikder et al. (Lancet, 2017), a randomised, double-blind, placebo-controlled trial of canakinumab in 10061 patients with atherosclerosis who had had a myocardial infarction, were free of previously diagnosed cancer, and had concentrations of high-sensitivity C-reactive protein (hsCRP) of 2 mg/L or greater was completed in June, 2017 (CANTOS trial). To assess dose-response effects, patients were randomly assigned by computer-generated codes to three canakinumab doses (50 mg, 150 mg, and 300 mg, subcutaneously every 3 months) or placebo.

Baseline concentrations of hsCRP (median 6.0 mg/L vs 4.2 mg/L; p<0.0001) and interleukin 6 (3.2 vs 2.6 ng/L; p<0.0001) were significantly higher among participants subsequently diagnosed with lung cancer than among those not diagnosed with cancer. During median follow-up of 3.7 years, compared with placebo, canakinumab was associated with dose-dependent reductions in concentrations of hsCRP of 26-41% and of interleukin 6 of 25-43% (p<0.0001 for all comparisons). Total cancer mortality (n=196) was significantly lower in the pooled canakinumab group than in the placebo group (p=0.0007 for trend across groups), but was significantly lower than placebo only in the 300 mg group individually (hazard ratio [HR]0 49 [95% CI 0.31-0.75]; p=0.0009). Incident lung cancer (n=129) was significantly less frequent in the 150 mg (HR 0 61 [95% CI 0.39-0.97]; p=0.034) and 300 mg groups (HR 0 33 [95% CI 0.18-0.59]; p<0.0001; p<0.0001 for trend across groups). Lung cancer mortality was significantly less common in the canakinumab 300 mg group than in the placebo group (HR 0.23 [95% CI 0.10-0-54]; p=0.0002) and in the pooled canakinumab population than in the placebo group (p=0.0002 for trend across groups).

Biomarker analysis of patients of non-lung cancers from the CANTOS trial, especially of the GI/GU cancers, has revealed that they have elevated baseline hsCRP level and IL-6 level. In addition, GI/GU cancer patients with higher baseline level of hsCRP and IL-6 seems to have a shorter time to cancer diagnosis than patients having lower baseline level (EXAMPLE 12), suggesting the likelihood of the involvement of IL-1β mediated inflammation in broader cancer indications, besides lung cancer, which warranties targeting IL-1β in the treatment of those cancers. In addition hsCRP level and IL-6 level in GI/GU patients were reduced in the range comparable to other patients in the CANTOS trial treatment group, suggesting inhibition of IL-1β signaling in those patients. Inhibition of IL-1β alone or preferably in combination with other anti-cancer agents could results in clinical benefit in treating cancer, e.g., cancer having at least partial inflammatory basis, as further supported by data presented in EXAMPLEs.

### Cancers, e.g., Cancers Having at Least a Partial Inflammatory Basis

Thus in one aspect, the present invention provides the use of an IL-1β binding antibody or a functional fragment thereof (for reason of simplicity, the term “an IL-1β binding antibody or a functional fragment thereof” is sometimes referred as “DRUG of the invention” in this application, which should be understood as identical term), suitably canakinumab or a functional fragment thereof (included in “DRUG of the invention”), gevokizumab or a functional fragment thereof (included in “DRUG of the invention”), for the treatment and/or prevention of cancers, e.g., cancers that have at least a partial inflammatory basis, e.g., a cancer described herein.

Advanced studies in delineating the interaction between tumor and the tumor microenvironment have revealed that chronic inflammation can promote tumor development, and tumor fuels inflammation to facilitate tumor progression and metastasis. The inflammatory microenvironment with cellular and non-cellular secreted factors provides a sanctuary for tumor progression by inducing angiogenesis; recruiting tumor promoting, immune suppressive cells and inhibiting immune effector cell mediated anti-tumor immune response. One of the major inflammatory pathways supporting tumor development and progression is IL-1β, a pro-inflammatory cytokine produced by tumor and tumor associated immune suppressive cells including neutrophils and macrophages in tumor microenvironment.

Accordingly, the present disclosure provides method of treating cancer using an IL-1β binding antibody or a functional fragment thereof, wherein such IL-1β binding antibodies or functional fragments thereof can reduce inflammation and/or improve tumor microenvironment, e.g., can inhibit IL-1β mediated inflammation and IL-1β mediated immune suppression in the tumor microenvironment. An example of using an IL-1β binding antibody in modulating the tumor microenvironment is shown in Example 5 herein. In some embodiments, an IL-1β binding antibody or a functional fragment thereof is used alone as a monotherapy. In some embodiments, an IL-1β binding antibody or a functional fragment thereof is used in combination with another therapy, such as with a check point inhibitor and/or with one or more chemotherapeutic agents. As discussed herein, inflammation can promote tumor development, an IL-1β binding antibody or a functional fragment thereof, either alone or in combination with another therapy, can be used to treat any cancer that can benefit from (in terms of clinical benefit) the reduced IL-1β mediated inflammation and/or improved tumor environment. Inflammation component is universally present, albeit to different degrees, in the cancer development.

As used herein, “cancer” is meant to include all types of cancerous growths or oncogenic processes, metastatic tissues or malignantly transformed cells, tissues, or organs, irrespective of histopathologic type or stage of invasiveness. Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. In one embodiment, the cancer is a melanoma, e.g., an advanced stage melanoma. Metastatic lesions of the aforementioned cancers can also be treated or prevented using the methods and compositions of the invention.

Exemplary cancers whose growth can be inhibited using the antibodies molecules disclosed herein include cancers typically responsive to immunotherapy. Non-limiting examples of preferred cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). Additionally, refractory or recurrent malignancies can be treated using the antibody molecules described herein.

Examples of other cancers that can be treated include myeloproliferative neoplasms, bone cancer, pancreatic cancer, skin cancer, cancer of the head or neck, cutaneous or intraocular malignant melanoma, uterine cancer, ovarian cancer, rectal cancer, anal cancer, gastro-esophageal, stomach cancer, liposarcoma, testicular cancer, uterine cancer, carcinoma of the fallopian tubes, carcinoma of the endometrium, carcinoma of the cervix, carcinoma of the vagina, carcinoma of the vulva, Merkel cell cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, multiple myeloma, myelodysplastic syndromes, cancer of the kidney or ureter, carcinoma of the renal pelvis, neoplasm of the central nervous system (CNS), primary CNS lymphoma, tumor angiogenesis, spinal axis tumor, brain stem glioma, glioblastoma, pituitary adenoma, Kaposi's sarcoma, epidermoid cancer, squamous cell cancer, T-cell lymphoma, environmentally induced cancers including those induced by asbestos (e.g., mesothelioma), and combinations of said cancers. In certain embodiments, the cancer is a skin cancer, e.g., a Merkel cell carcinoma or a melanoma. In one embodiment, the cancer is a Merkel cell carcinoma. In other embodiments, the cancer is a melanoma. In other embodiments, the cancer is a breast cancer, e.g., a triple negative breast cancer (TNBC) or a HER2-negative breast cancer. In other embodiments, the cancer is kidney cancer, e.g., a renal cell carcinoma (e.g., clear cell renal cell carcinoma (CCRCC) or a non-clear cell renal cell carcinoma (nccRCC)). In other embodiments, the cancer is a thyroid cancer, e.g., an anaplastic thyroid carcinoma (ATC). In other embodiments, the cancer is a neuroendocrine tumor (NET), e.g., an atypical pulmonary carcinoid tumor or an NET in pancreas, gastrointestinal (GI) tract, or lung. In certain embodiments, the cancer is a lung cancer, e.g., a non-small cell lung cancer (NSCLC) (e.g., a squamous NSCLC or a non-squamous NSCLC). In certain embodiments, the cancer is a leukemia (e.g., an acute myeloid leukemia (AML), e.g., a relapsed or refractory AML or a de novo AML). In certain embodiments, the cancer is a myelodysplastic syndrome (MDS) (e.g., a high risk MDS).

In some embodiments, the cancer is chosen from a lung cancer, a squamous cell lung cancer, a melanoma, a renal cancer, a liver cancer, a myeloma, a prostate cancer, a breast cancer, an ER+ breast cancer, an IM-TN breast cancer, a colorectal cancer, a colorectal cancer with high microsatellite instability, an EBV+ gastric cancer, a pancreatic cancer, a thyroid cancer, a hematological cancer, a non-Hogdkin's lymphoma, or a leukemia, or a metastatic lesion of the cancer. In some embodiments, the cancer is chosen from a non-small cell lung cancer (NSCLC), a NSCLC adenocarcinoma, a NSCLC squamous cell carcinoma, or a hepatocellular carcinoma.

The meaning of “cancers that have at least a partial inflammatory basis” or “cancer having at least a partial inflammatory basis” is well known in the art and as used herein refers to any cancer in which IL-1β mediated inflammatory responses contribute to tumor development and/or propagation, including but not necessarily limited to metastasis. Such cancer generally has concomitant inflammation activated or mediated in part through activation of the Nod-like receptor protein 3 (NLRP3) inflammasome with consequent local production of interleukin-1β. In a patient with such cancer, the expression, or even the overexpression of IL-1β can be generally detected, commonly at the site of the tumor, especially in the surrounding tissue of the tumor, in comparison to normal tissue. The expression of IL-1β can be detected by routine methods known in the art, such as immunostaining, ELISA based assays, ISH, RNA sequencing or RT-PCR in the tumor as well as in serum/plasma. The expression or higher expression of IL-1β can be concluded, for example, against negative control, usually normal tissue at the same site or can be concluded if it is higher than normal level of IL-1β in serum/plasma of a healthy person (reference level). Simultaneously or alternatively, a patient with such cancer has generally chronic inflammation, which is manifested, typically, by higher than normal level of hsCRP (or CRP), IL-6 or TNFα, preferably by hsCRP or IL-6, preferably by IL-6. This is because IL-6 is immediate downstream of IL-1β. hsCRP is further downstream and can be influenced by other factors as well. Cancers, particularly cancers that have at least a partial inflammatory basis, include but not limited to lung cancer, particularly NSCLC, colorectal cancer, melanoma, gastric cancer (including gastric and intestinal cancer, cancer of the esophagus, particularly the lower part of the esophagus, renal cell carcinoma (RCC), breast cancer, prostate cancer, head and neck cancer (including oral cancer, including HPV, EBV and tobacco and/or alcohol induded head and neck cancer), bladder cancer, liver cancer such as hepatocellular carcinoma (HCC), pancreatic cancer, especially pancreatic ductal adenocarcinoma (PDAC), ovarian cancer, cervical cancer, endometrial cancer, neuroendocrine cancer and biliary tract cancer (including but not limited to bile duct and gallbladder cancers) and hematologic cancers such as acute myeloblastic leukemia (AML), myelofibrosis and multiple myeloma (MM). Cancers also include cancers that may not express IL-1β until after previous treatment of such cancer, e.g., including treatment with a chemotherapeutic agent, e.g., as described herein, which contribute to the expression of IL-1B in the tumor and/or tumor microenvironment. In some embodiments, the methods and use comprise treating a patient whose cancer is relapsed or recurring after treatment with such agent. In other embodiments, the agent is associated with IL-1β expression and the IL-1β antibody or functional fragment thereof is given in combination with such agent.

Inhibition of IL-1β resulted in reduced inflammation status, including but not limited to reduced hsCRP or IL-6 level. Thus the effect of the present invention in cancer patients can be measured by reduced inflammation status, including but not limited to reduced hsCRP or IL-6 level.

The term “cancers that have at least a partial inflammatory basis” or “cancer having at least a partial inflammatory basis” also includes cancers that benefit from the treatment of an IL-1β binding antibody or a functional fragment thereof. As inflammation in general contributes to tumor growth at already an early stage, administration of IL-1β binding antibody or a functional fragment thereof (canakinumab or gevokizumab) could potentially stop tumor growth effectively at the early stage or delay tumor progression effectively at the early stage, even though the inflammation status, such as expression or overexpression IL-1β, or the elevated level of CRP or hsCRP, IL-6 or TNFα, is still not apparent or measurable. Furthermore in patient whose cancer has just been resected, the inflammation could be reduced, shown by lowered IL-1β, hsCRP, IL-6 or TNFα level. However patients having early stage cancers or patients have tumor removed still can benefit from the treatment of IL-1β binding antibody or a functional fragment, which can be shown in clinical trials. The clinical benefit can be measured by, including but not limited to, disease-free survival (DFS), progression-free survival (PFS), Overall response rate (ORR), disease control rate (DCR), duration of response (DOR) and overall survival (OS), preferably in a clinical trial setting, against proper control group, for example against the effects achieved by standard of care (SoC) drugs, either by added on top of SoC or without SoC. If a patient treated with the DRUG of the invention has shown any improvement in one or more of the above parameters in comparison to the control, the patient is considered to have benefited from the treatment according to the present invention. Accordingly the cancer that benefit from an IL-1β binding antibody or a functional fragment thereof (canakinumab or gevokizumab) treatment is considered as cancer having at least partial inflammatory basis.

The term “Overall survival (OS)” is typically defined as the time of randomization to death due to any cause. Patients still alive at the time of an analysis will be considered censored at their date of last contact.

The term “Progression-free survival (PFS)” is typically defined as the time from randomization to clinically determined progression or death from any cause.

The term “Overall tumor response (ORR)” includes both complete response (CR) and partial response (PR).

The term “Duration of ORR” is typically defined as the time from the date of response to the date of clinically determined disease progression or death from any cause.

Available techniques known to the skilled person in the art allow detection and quantification of IL-1β in tissue as well as in serum/plasma, particularly when the IL-1β is expressed to a higher than normal level. For example, Using the R&D Systems high sensitivity IL-1β ELISA kit, IL-1β cannot be detected in majority of healthy donor serum samples, as shown in the following Table 1.

TABLE 1
SAMPLE VALUES
Serum/Plasma-Samples from apparently healthy
volunteers were evaluated for the presence
of human IL-1β in this assay. No medical histories
were available for the donors used in this study.

| | Mean of | % | Range |
|---|---|---|---|
| Sample Type | Detectable (pg/mL) | Detectable | (pg/mL) |
| Serum (n = 50) | 0.357 | 10 | ND-0.606 |
| EDTA plasma (n = 50) | 0.292 | 12 | ND-0.580 |
| Heparin plasma (n = 50) | 0.448 | 14 | ND-1.08 |

ND = Non-detectable

Thus in a healthy person the IL-1β level is barely detectable or just above the detection limit according to this test with the high sensitivity R&D® IL-1β ELISA kit. It is expected that in a patient with cancer having at least partial inflammatory basis in general has higher than normal level of IL-1β and can be detected by the same kit. Taking the IL-1β expression level in a healthy person as the normal level (reference level), the term “higher than normal level of IL-1β” means an IL-1β level that is higher than the reference level. Normally at least about 2 fold, at least about 5 fold, at least about 10 fold of the reference level is considered as higher than normal level. Alternatively taking the IL-1β expression level in a healthy person as the normal level (reference level), the term “higher than normal level of IL-1β” means an IL-1β level that is higher than the reference level, normally higher than about 0.8 pg/ml, higher than about 1 pg/ml, higher than about 1.3 pg/ml, higher than about 1.5 pg/ml, higher than about 2 pg/ml, higher than about 3 pg/ml, as determined preferably by the R&D kit mentioned above. Blocking the IL-1β pathway normally triggers the compensating mechanism leading to more production of IL-1β. Thus the term “higher than normal level of IL-1β” also means and includes the level of IL-1β either post, or more preferably, prior to the administration of an IL-1β binding antibody or a fragment thereof. Treatment of cancer with agents other than IL-1β inhibitors, such as some chemotherapeutic agents, can result in production of IL-1β in the tumor microenvironment. Thus the term “higher than normal level of IL-1β” also refers to the level of IL-1β either prior to or post the administration of such an agent.

When using staining, such as immunostaining, to detect IL-1β expression in a tissue preparation, the term “higher than normal level of IL-1β” means that the staining signal generated by specific IL-1β protein or IL-1β RNA detecting molecule is distinguishably stronger than staining signal of the surrounding tissue not expressing IL-1β.

Available techniques known to the skilled person in the art allow detection and quantification of IL-6 in tissue as well as in serum/plasma, particularly when the IL-6 is expressed to a higher than normal level. For example, using the R&D Systems (world wide web.R&DSystems.com) “high quantikine HS ELISA, human IL-6 Immnunoassay”, IL-6 can be detected in majority of healthy donor serum samples, as shown in the following Table 2.

TABLE 2
SAMPLE VALUES
Samples from apparently healthy volunteers were
evaluated for the presence of human IL-6 in this assay.
No medical histories were available for the
donors used in this study.

| | Mean of | % | Range |
|---|---|---|---|
| Sample Type | Detectable (pg/mL) | Detectable | (pg/mL) |
| Serum (n = 52) | 1.77 | 100 | 0.447-9.96 |
| EDTA plasma (n = 35) | 1.49 | 100 | 0.428-8.87 |
| Citrate plasma (n = 16) | 1.57 | 100 | 0.435-9.57 |
| Urine (n = 14) | 1.67 | 93 | ND-6.76 |

ND = Non-detectable

It is expected that in a patient with cancer having at least partial inflammatory basis in general has higher than normal level of IL-6 and can be detected by the same kit. Taking the IL-6 expression level in a healthy person as the normal level (reference level), the term “higher than normal level of IL-6” means an IL-6 level that is higher than the reference level, normally higher than about 1.9 pg/ml, higher than about 2 pg/ml, higher than about 2.2 pg/ml, higher than about 2.5 pg/ml, higher than about 2.7 pg/ml, higher than about 3 pg/ml, higher than about 3.5 pg/ml, or higher than about 4 pg/ml, as determined preferably by the R&D kit mentioned above. Blocking the IL-1β pathway normally triggers the compensating mechanism leading to more production of IL-1β3. Thus the term “higher than normal level of IL-6” also means and includes the level of IL-6 either post, or more preferably, prior to the administration of an IL-1β binding antibody or a fragment thereof. Treatment of cancer with agents other than IL-1β3 inhibitors, such as some chemotherapeutic agents, can result in production of IL-1β3 in the tumor microenvironment. Thus the term “higher than normal level of IL-6” also refers to the level of IL-6 either prior to or post the administration of such an agent.

When using staining, such as immunostaining, to detect IL-6 expression in a tissue preparation, the term “higher than normal level of IL-6” means that the staining signal generated by specific IL-6 protein or IL-6 RNA detecting molecule is distinguishably stronger than staining signal of the surrounding tissue not expressing IL-6.

As used herein, the terms “treat”, “treatment” and “treating” refer to the reduction or amelioration of the progression, severity and/or duration of a disorder, e.g., a proliferative disorder, or the amelioration of one or more symptoms, suitably of one or more discernible symptoms, of the disorder resulting from the administration of one or more therapies. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count. As far as cancers as discussed here, taking lung cancer as an example, the term treatment refers to at least one of the following: alleviating one or more symptoms of lung cancer, delaying progression of lung cancer, shrinking tumor size in lung cancer patient, inhibiting lung cancer tumor growth, prolonging overall survival, prolonging progression free survival, preventing or delaying lung cancer tumor metastasis, reducing (such as eradiating) pre-existing lung cancer tumor metastasis, reducing incidence or burden of pre-existing lung cancer tumor metastasis, or preventing recurrence of lung cancer.

In one embodiment, cancers, e.g., cancers having at least partial inflammatory basis, is selected from a list consisting of lung cancer, especially NSCLC, colorectal cancer (CRC), melanoma, gastric cancer (including esophageal cancer), renal cell carcinoma (RCC), breast cancer, prostate cancer, head and neck cancer (including oral), bladder cancer, hepatocellular carcinoma (HCC), ovarian cancer, cervical cancer, pancreatic cancer, especially PDAC, hematological cancer (particularly multiple myeloma, acute myeloblastic leukemia (AML).

In one embodiment, cancers, e.g., cancers having at least partial inflammatory basis, is selected from a list consisting of lung cancer, especially NSCLC, colorectal cancer (CRC), melanoma, gastric cancer (including esophageal cancer), renal cell carcinoma (RCC), breast cancer, prostate cancer, head and neck cancer (including oral), bladder cancer, hepatocellular carcinoma (HCC), ovarian cancer, cervical cancer, pancreatic cancer, especially PDAC.

In one embodiment, cancers, e.g., cancers having at least partial inflammatory basis, is selected from a list consisting of lung cancer, especially NSCLC, colorectal cancer (CRC), melanoma, gastric cancer (including esophageal cancer), renal cell carcinoma (RCC), breast cancer, prostate cancer, head and neck cancer (including oral), bladder cancer, cervical cancer, pancreatic cancer, especially PDAC.

In one embodiment, cancers, e.g., cancers having at least partial inflammatory basis, is a cancer from the gastrointestinal tract, including but not limited to gastric cancer (including esophageal cancer), CRC and pancreatic cancer, especially PDAC. In one embodiment, cancers, e.g., cancers having at least partial inflammatory basis, is a cancer from the genitourinary system, including but not limited to RCC, bladder cancer and prostate cancer.

### IL-1β Inhibitors, Especially IL-1β Binding Antibody or a Fragment Thereof

As used herein, IL-1β inhibitors include but are not limited to, canakinumab or a functional fragment thereof, gevokizumab or a functional fragment thereof, Anakinra, diacerein, Rilonacept, IL-1 Affibody (SOBI 006, Z-FC (Swedish Orphan Biovitrum/Affibody)) and Lutikizumab (ABT-981) (Abbott), CDP-484 (Celltech), LY-2189102 (Lilly).

In one embodiment, of any use or method of the invention, said IL-1β binding antibody is canakinumab. Canakinumab (ACZ885) is a high-affinity, fully human monoclonal antibody of the IgG1/k to interleukin-1β, developed for the treatment of IL-1β driven inflammatory diseases. It is designed to bind to human IL-1β and thus blocks the interaction of this cytokine with its receptors.

In other embodiments of any use or method of the invention, said IL-1β binding antibody is gevokizumab. Gevokizumab (XOMA-052) is a high-affinity, humanized monoclonal antibody of the IgG2 isotype to interleukin-1β, developed for the treatment of IL-1β driven inflammatory diseases. Gevokizumab modulates IL-1β binding to its signaling receptor.

In one embodiment, said IL-1β binding antibody is LY-2189102, which is a humanised interleukin-1 beta (IL-1β) monoclonal antibody.

In one embodiment, said IL-1β binding antibody or a functional fragment thereof is CDP-484 (Celltech), which is an antibody fragment blocking IL-1β.

In one embodiment, said IL-1β binding antibody or a functional fragment thereof is IL-1 Affibody (SOBI 006, Z-FC (Swedish Orphan Biovitrum/Affibody)).

An antibody, as used herein, refers to an antibody having the natural biological form of an antibody. Such an antibody is a glycoprotein and consists of four polypeptides—two identical heavy chains and two identical light chains, joined to form a “Y”-shaped molecule. Each heavy chain is comprised of a heavy chain variable region (VH) and a heavy chain constant region. The heavy chain constant region is comprised of three or four constant domains (CH1, CH2, CH3, and CH4, depending on the antibody class or isotype). Each light chain is comprised of a light chain variable region (VL) and a light chain constant region, which has one domain, CL. Papain, a proteolytic enzyme, splits the “Y” shape into three separate molecules, two so called “Fab” fragments (Fab=fragment antigen binding), and one so called “Fc” fragment (Fc=fragment crystallizable). A Fab fragment consists of the entire light chain and part of the heavy chain. The VL and VH regions are located at the tips of the “Y”-shaped antibody molecule. The VL and VH each have three complementarity-determining regions (CDRs).

By “IL-1β binding antibody” is meant any antibody capable of binding to the IL-1β specifically and consequently inhibiting or modulating the binding of IL-1β to its receptor and further consequently inhibiting IL-1β function. Preferably an IL-1β binding antibody does not bind to IL-1α.

Preferably an IL-1β binding antibody includes:

(1) An antibody comprising three VL CDRs having the amino acid sequences RASQSIGSSLH (SEQ ID NO: 1), ASQSFS (SEQ ID NO: 2), and HQSSSLP (SEQ ID NO: 3) and three VH CDRs having the amino acid sequences VYGMN (SEQ ID NO: 5), IIWYDGDNQYYADSVKG (SEQ ID NO: 6), and DLRTGP (SEQ ID NO: 7);

(2) An antibody comprising three VL CDRs having the amino acid sequences RASQDISNYLS (SEQ ID NO: 9), YTSKLHS (SEQ ID NO: 10), and LQGKMLPWT (SEQ ID NO: 11), and three VH CDRs having the amino acid sequences TSGMGVG (SEQ ID NO: 13), HIWWDGDESYNPSLK (SEQ ID NO: 14), and NRYDPPWFVD (SEQ ID NO: 15); and

(3) An antibody comprising the six CDRs as described in either (1) or (2), wherein one or more of the CDR sequences, preferably at most two of the CDRs, preferably only one of the CDRs, differ by one amino acid from the corresponding sequences described in either (1) or (2), respectively.

Preferably an IL-1β binding antibody includes:

(1) An antibody comprising three VL CDRs having the amino acid sequences RASQSIGSSLH (SEQ ID NO: 1), ASQSFS (SEQ ID NO: 2), and HQSSSLP (SEQ ID NO: 3) and comprising the VH having the amino acid sequence specified in SEQ ID NO: 8;

(2) An antibody comprising the VL having the amino acid sequence specified in SEQ ID NO: 4 and comprising three VH CDRs having the amino acid sequences VYGMN (SEQ ID NO: 5), IIWYDGDNQYYADSVKG (SEQ ID NO: 6), and DLRTGP (SEQ ID NO: 7);

(3) An antibody comprising three VL CDRs having the amino acid sequences RASQDISNYLS (SEQ ID NO: 9), YTSKLHS (SEQ ID NO: 10), and LQGKMLPWT (SEQ ID NO: 11), and comprising the VH having the amino acid sequences specified in SEQ ID NO: 16;

(4) An antibody comprising the VL having the amino acid specified in SEQ ID NO: 12, and comprising three VH CDRs having the amino acid sequences TSGMGVG (SEQ ID NO: 13), HIWWDGDESYNPSLK (SEQ ID NO: 14), and NRYDPPWFVD (SEQ ID NO: 15);

(5) An antibody comprising three VL CDRs and the VH sequence as described in either (1) or (3), wherein one or more of the VL CDR sequences, preferably at most two of the CDRs, preferably only one of the CDRs, differ by one amino acid from the corresponding sequences described in (1) or (3), respectively, and wherein the VH sequence is at least 90% identical to the corresponding sequence described in (1) or (3), respectively; and

(6) An antibody comprising the VL sequence and three VH CDRs as described in either (2) or (4), wherein the VL sequence is at least 90% identical to the corresponding sequence described in (2) or (4), respectively, and wherein one or more of the VH CDR sequences, preferably at most two of the CDRs, preferably only one of the CDRs, differ by one amino acid from the corresponding sequences described in (2) or (4), respectively.

Preferably an IL-1β binding antibody includes:

(1) An antibody comprising the VL having the amino acid sequence specified in SEQ ID NO: 4 and comprising the VH having the amino acid sequence specified in SEQ ID NO: 8;

(2) An antibody comprising the VL having the amino acid specified in SEQ ID NO: 12, and comprising the VH having the amino acid sequences specified in SEQ ID NO: 16; and

(3) An antibody described in either (1) or (2), wherein the constant region of the heavy chain, the constant region of the light chain or both has been changed to a different isotype as compared to canakinumab or gevokizumab.

Preferably an IL-1β binding antibody includes:

(1) Canakinumab (SEQ ID NO:17 and 18); and

(2) Gevokizumab (SEQ ID NO:19 and 20).

An IL-1β binding antibody as defined above has substantially identical or identical CDR sequences as those of canakinumab or gevokizumab. It thus binds to the same epitope on IL-1β and has similar binding affinity as canakinumab or gevokizumab. The clinical relevant doses and dosing regimens that have been established for canakinumab or gevokizumab as therapeutically efficacious in the treatment of cancer, especially cancer having at least partial inflammatory basis, would be applicable to other IL-1β binding antibodies.
Additionally or alternatively, an IL-1β antibody refers to an antibody that is capable of binding to IL-1β specifically with affinity in the similar range as canakinumab or gevokizumab. The Kd for canakinumab in WO2007/050607 is referenced with 30.5 pM, whereas the Kd for gevokizumab is 0.3 pM. Thus affinity in the similar range refers to between about 0.05 pM to 300 pM, preferably 0.1 pM to 100 pM. Although both binding to IL-IP, canakinumab directly inhibits the binding to IL-1 receptor, whereas gevokizumab is an allosteric inhibitor. It does not prevent IL-1β from binding to the receptor but prevent receptor activation. Preferably an IL-1β antibody has the binding affinity in the similar range as canakinumab, preferably in the range of 1 pM to 300 pM, preferably in the range of 10 pM to 100 pM, wherein preferably said antibody directly inhibits binding. Preferably an IL-1β antibody has the binding affinity in the similar range as gevokizumab, preferably in the range of 0.05 pM to 3 pM, preferably in the range of 0.1 pM to 1 pM, wherein preferably said antibody is an allosteric inhibitor.

As used herein, the term “functional fragment” of an antibody as used herein, refers to portions or fragments of an antibody that retain the ability to specifically bind to an antigen (e.g., IL-1β). Examples of binding fragments encompassed within the term “functional fragment” of an antibody include single chain Fv (scFv), a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989), which consists of a V_{H} domain; and an isolated complementarity determining region (CDR); and one or more CDRs arranged on peptide scaffolds that can be smaller, larger, or fold differently to a typical antibody.

The term “functional fragment” might also refer to one of the following:
bispecific single chain Fv dimers (PCT/US92/09965)
“diabodies” or “triabodies”, multivalent or multispecific fragments constructed by gene fusion (Tomlinson I & Hollinger P (2000) Methods Enzymol. 326: 461-79; WO94113804; Holliger P et al., (1993) Proc. Natl. Acad. Sci. USA, 90: 6444-48)
scFv genetically fused to the same or a different antibody (Coloma M J & Morrison S L (1997) Nature Biotechnology, 15(2): 159-163)
scFv, diabody or domain antibody fused to an Fc region
scFv fused to the same or a different antibody
Fv, scFv or diabody molecules may be stabilized by the incorporation of disulphide bridges linking the VH and VL domains (Reiter, Y. et al, (1996) Nature Biotech, 14, 1239-1245).
Minibodies comprising a scFv joined to a CH3 domain may also be made (Hu, S. et al, (1996) Cancer Res., 56, 3055-3061).
Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group

Typically and preferably an functional fragment of an IL-1β binding antibody is a portion or a fragment of an “IL-1β binding antibody” as defined above.

### Dosing Regimen of the Present Invention

If an IL-1β inhibitor, such as an IL-1β antibody or a functional fragment thereof, is administered in a dose range that can effectively reduce hsCRP level in a patient with cancer having at least partial inflamatory basis, treatment effect of said cancer can possibly be achieved. Dose range, of a particular IL-1β inhibitor, preferably IL-1β antibody or a functional fragment thereof, that can effectively reduce hsCRP level is known or can be tested in a clinical setting.

Thus In one embodiment, the present invention comprises administering the IL-1β binding antibody or a functional fragment thereof to a patient with cancer, e.g., cancer that has at least a partial inflammatory basis, in the range of about 20 mg to about 400 mg per treatment, preferably in the range of about 30 mg to about 400 mg per treatment, preferably in the range of about 30 mg to about 200 mg per treatment, preferably in the range of about 60 mg to about 200 mg per treatment. In one embodiment, the patient receives each treatment every two weeks, every three weeks, every four weeks (monthly), every 6 weeks, bimonthly (every 2 months), every nine weeks or quarterly (every 3 months). In one embodiment, patient receives each treatment every 3 weeks. In one embodiment, patient receives each treatment every 4 weeks. The term “per treatment”, as used in this application and particularly in this context, should be understood as the total amount of drug received per hospital visit or per self administration or per administration helped by a health care giver. Normally and preferably the total amount of drug received per treatment is administered to a patient is within 2 hours, preferably within one hour, or within half hour. In one preferred embodiment the term “per treatment” is understood as the drug is administered with one injection, preferably in one dosage.

In practice some times the time interval can not be strictly kept due to the limitation of the availability of doctor, patient or the drug/facility. Thus the time interval can slightly vary, normally between ±5 days, +4 days, +3 days, +2 days or preferably ±1 day.

Some times it is desirable to quickly reduce inflammation. IL-1β auto-induction has been shown in human mononuclear blood, human vascular endothelial, and vascular smooth muscle cells in vitro and in rabbits in vivo where IL-1 has been shown to induce its own gene expression and circulating IL-1β level (Dinarello et al. 1987, Warner et al. 1987a, and Warner et al. 1987b).

This induction period over 2 weeks by administration of a first dose followed by a second dose two weeks after administration of the first dose is to assure that auto-induction of IL-1β pathway is adequately inhibited at initiation of treatment. The complete suppression of IL-1β related gene expression achieved with this early high dose administration, coupled with the continuous canakinumab treatment effect which has been proven to last the entire quarterly dosing period used in CANTOS, is to minimize the potential for IL-1β rebound. In addition, data in the setting of acute inflammation suggests that higher initial doses of canakinumab that can be achieved through induction are safe and provide an opportunity to ameliorate concern regarding potential auto-induction of IL-1β and to achieve greater early suppression of IL-1β related gene expression.

Thus In one embodiment, the present invention, while keeping the above described dosing schedules, especially envisages the second administration of DRUG of the invention is one weel later or at most two weeks, preferably two weeks apart from the first administration. Then the third and the further administration will following the schedule of every 2 weeks, every 3 weeks, every 4 weeks (monthly), every 6 weeks, bimonthly (every 2 months), every 9 weeks or quarterly (every 3 months).

In one embodiment, the IL-1β binding antibody is canakinumab, wherein canakinumab is administered to a patient with cancer, e.g., cancer that has at least a partial inflammatory basis, in the range of about 100 mg to about 400 mg, preferably about 200 mg per treatment. In one embodiment, the patient receives each treatment every 2 weeks, every 3 weeks, every 4 weeks (monthly), every 6 weeks, bimonthly (every 2 months), every 9 weeks or quarterly (every 3 months). In one embodiment, the patient receives canakinumab monthly or every three weeks. In one embodiment, the preferred dose of canakinumab for patient is about 200 mg every 3 weeks. In one embodiment, the preferred dose of canakinumab for is about 200 mg monthly. When safety concern raises, the dose can be down-titrated, preferably by increasing the dosing interval, preferably by doubling or tripling the dosing interval. For example about 200 mg monthly or every 3 weeks regimen can be changed to every 2 month or every 6 weeks respectively or every 3 month or every 9 weeks respectively. In an alternative embodiment the patient receives canakinumab at a dose of about 200 mg every two month or every 6 weeks in the down-titration phase or in the maintenance phase independent from any safety issue or throughout the treatment phase. In an alternative embodiment the patient receives canakinumab at a dose of 200 mg every 3 month or every 9 weeks in the down-titration phase or in the maintenance phase independent from any safety issue or throughout the treatment phase.

In one embodiment canakinumab is administered to a patient with cancer, e.g., cancer that has at least a partial inflammatory basis, in the range of about 100 mg to about 400 mg, in the range of 150 mg to 300 mg per treatment, suitably 250 mg per treatment. preferably about 200 mg per treatment, every 2 weeks, every 3 weeks, every 4 weeks (monthly), every 6 weeks, bimonthly (every 2 months), every 9 weeks or quarterly (every 3 months). In one embodiment canakinumab is administered 250 mg per treatment every 4 weeks (monthly).

Suitably the above dose and dosing apply to the use of a functional fragment of canakinumab according to the present invention.

Canakinumab or a functional fragment thereof can be administered intravenously or subcutaneously, preferably subcutaneously.

The dosing regimens disclosed herein is applicable in each and every canakinumab related embodiments disclosed in this application, including but not limited to monotherapy or in combination with one or more anti-cancer therapeutic agents, used in adjuvant setting or in the first line, 2^{nd} line or 3^{rd} line treatment.

In one embodiment, the present invention comprises administering gevokizumab to a patient with cancer, e.g., cancer that has at least a partial inflammatory basis, in the range of about 20 mg to about 240 mg per treatment, preferably in the range of about 20 mg to about 180 mg, preferably in the range of about 30 mg to about 120 mg, preferably about 30 mg to about 60 mg, preferably about 60 mg to about 120 mg per treatment. In one embodiment, patient receives about 30 mg to about 120 mg per treatment. In one embodiment, patient receives about 30 mg to about 60 mg per treatment. In one embodiment, patient receives about 30 mg, 60 mg, 90 mg, 120 mg or 180 mg per treatment. In one embodiment, the patient receives each treatment every 2 weeks, every 3 weeks, monthly (every 4 weeks), every 6 weeks, bimonthly (every 2 months), every 9 weeks or quarterly (every 3 months). In one embodiment, the patient receives each treatment every 3 weeks. In one embodiment, the patient receives each treatment every 4 weeks.

When safety concern raises, the dose can be down-titrated, preferably by increasing the dosing interval, preferably by doubling or tripling the dosing interval. For example 60 mg monthly or every 3 weeks regimen can be doubled to every 2 month or every 6 weeks respectively or tripled to every 3 month or every 9 weeks respectively. In an alternative embodiment the patient receives gevokizumab at a dose of about 30 mg to about 120 mg every 2 month or every 6 weeks in the down-titration phase or in the maintenance phase independent from any safety issue or throughout the treatment phase. In an alternative embodiment the patient receives gevokizumab at a dose of about 30 mg to about 120 mg every 3 month or every 9 weeks in the down-titration phase or in the maintenance phase independent from any safety issue or throughout the treatment phase.

Suitably the above dose and dosing apply to the use of a functional fragment of gevokizumab according to the present invention.

Gevokizumab or a functional fragment thereof can be administered intravenously or subcutaneously, preferably intravenously.

The dosing regimens disclosed herein is applicable in each and every gevokizumab related embodiments disclosed in this application, including but not limited to monotherapy or in combination with one or more anti-cancer therapeutic agents, used in adjuvant setting or in the first line, 2^{nd} line or 3^{rd} line treatment.

When canakinumab or gevokizumab is used in combination with one or more anti-cancer therapeutical agents, e.g., a chemotherapeutic agent or a check point inhibitor, especially when the one or more therapeutical agents is the SoC of the cancer indication, the dosing interval of canakinumab or gevokizumab can be adjusted to be aligned with the combination partner for the sake of patient convenience. Normally there is no need to change the canakinumab or gevokizumab dose per treatment. For example canakinumab 200 mg is administered every 3 weeks in combination with pembrolizumab, for example in NSCLC. For example canakinumab 200 mg is administered every 4 weeks in combination with FOLFOX, for example in CRC.

### Biomarkers

In one aspect, the present invention provides the use of an IL-1β binding antibody or a functional fragment thereof, suitably canakinumab or gevokizumab, in the treatment and/or prevention of cancer, e.g., cancer having at least a partial inflammatory basis, in a patient who has a higher than normal level of C-reactive protein (hsCRP). In one further embodiment, this patient is a smoker. In one further embodiment, the patient is a current smoker. Typically cancers, e.g., cancers that have at least a partial inflammatory basis, that possibly have patients exhibiting higher than normal hsCRP levels include, but are not limited to, lung cancer, especially NSCLC, colorectal cancer (CRC), melanoma, gastric cancer (including esophageal cancer), renal cell carcinoma (RCC), breast cancer, prostate cancer, head and neck cancer (including oral), bladder cancer, hepatocellular carcinoma (HCC), ovarian cancer, cervical cancer, pancreatic cancer, especially PDAC and multiple myeloma.

As used herein, “C-reactive protein” and “CRP” refers to serum or plasma C-reactive protein, which is typically used as an indicator of the acute phase response to inflammation. Nonetheless, CRP level may become elevated in chronic illnesses such as cancer. The level of CRP in serum or plasma may be given in any concentration, e.g., mg/dl, mg/L, nmol/L. Levels of CRP may be measured by a variety of wellknown methods, e.g., radial immunodiffusion, electroimmunoassay, immunoturbidimetry (e.g., particle (e.g., latex)-enhanced turbidimetric immunoassay), ELISA, turbidimetric methods, fluorescence polarization immunoassay, and laser nephelometry. Testing for CRP may employ a standard CRP test or a high sensitivity CRP (hsCRP) test (i.e., a high sensitivity test that is capable of measuring lower levels of CRP in a sample, e.g., using immunoassay or laser nephelometry). Kits for detecting levels of CRP may be purchased from various companies, e.g., Calbiotech, Inc, Cayman Chemical, Roche Diagnostics Corporation, Abazyme, DADE Behring, Abnova Corporation, Aniara Corporation, Bio-Quant Inc., Siemens Healthcare Diagnostics, Abbott Laboratories etc.

As used herein, the term “hsCRP” refers to the level of CRP in the blood (serum or plasma) as measured by high sensitivity CRP testing. For example, Tina-quant C-reactive protein (latex) high sensitivity assay (Roche Diagnostics Corporation) may be used for quantification of the hsCRP level of a subject. Such latex-enhanced turbidimetric immunoassay may be analysed on the Cobas® platform (Roche Diagnostics Corporation) or Roche/Hitachi (e.g., Modular P) analyzer. In the CANTOS trial the hsCRP level was measured by Tina-quant C-reactive protein (latex) high sensitivity assay (Roche Diagnostics Corporation) on the Roche/Hitachi Modular P analyzer, which can be used typically and preferably as the method in measuring hsCRP level. Alternatively the hsCRP level can be measured by another method, for example by another approved companion diagnostic kit, the value of which can be calibrated against the value measured by the Tina-quant method.

Each local laboratory employ a cut-off value for abnormal (high) CRP or hsCRP based on that laboratory's rule for calculating normal maximum CRP, i.e. based on that laboratory's reference standard. A physician generally orders a CRP test from a local laboratory, and the local laboratory determines CRP or hsCRP value and reports normal or abnormal (low or high) CRP using the rule that particular laboratory employs to calculate normal CRP, namely based on its reference standard. Thus whether a patient has a higher than normal level of C-reactive protein (hsCRP) can be determined by the local laboratory where the test is conducted.

It is plausible that an IL-1β antibody or a fragment thereof, such as canakinumab or gevokizumab, is effective in treating and/or preventing other cancer having at least partially inflammatory basis in a patient, especially when said patient has higher than normal level of hsCRP. Like canakinumab, gevokizumab binds to IL-1β specifically. Unlike canakinumab directly inhibiting the binding of IL-1β to its receptor, gevokizumab is an allosteric inhibitor. It does not inhibit IL-1β from binding to its receptor but prevent the receptor from being activated by IL-1β. Like canakinumab, gevokizumab was tested in a few inflammation based indications and has been shown to effectively reduce inflammation as indicated, for example, by the reduction of hsCRP level in those patients. Furthermore from the available IC50 value, gevokizumab seems to be a more potent IL-1β inhibitor than canakinumab.

Furthermore, the present invention provides effective dosing ranges, within which the HsCRP level can be reduced to certain threshold, below which more patients with cancer having at least partially inflammatory basis can become responder or below which the same patient can benefit more from the great therapeutic effect of the Drug of the invention with negligible or tolerable side effects.

In one aspect, the present invention provides high sensitivity C-reactive protein (hsCRP) or CRP for use as a biomarker in the treatment and/or prevention of cancer, e.g., cancer having at least a partial inflammatory basis, with an IL-1β inhibitor, e.g., IL-1β binding antibody or a functional fragment thereof. The level of hsCRP is possibly relevant in determining whether a patient with diagnosed or undiagnosed cancer or is at risk of developing cancer should be treated with an IL-1β binding antibody or a functional fragment thereof. In one embodiment, patient is eligible for the treatment and/or prevention if the level of hsCRP is equal to or higher than 2.5 mg/L, or equal to or higher than 4.5 mg/L, or equal to or higher than 7.5 mg/L, or equal to or higher than 9.5 mg/L, as assessed prior to the administration of the IL-1R binding antibody or a functional fragment thereof.

In one embodiment, the present invention provides the use of an IL-1β binding antibody or a functional fragment thereof, suitably canakinumab or gevokizumab, for the treatment and/or prevention of cancer, e.g., cancer that has at least a partial inflammatory basis, in a patient who has high sensitivity C-reactive protein (hsCRP) level equal to or higher than about 2.2 mg/L, equal to or higher than about 4.2 mg/L, equal to or higher than about 6.2 mg/L equal to or higher than about 10.2 mg/L, preferably before first administration of said IL-1β binding antibody or functional fragment thereof. Preferably said patient has a hsCRP level equal to or higher than about 4.2 mg/L. Preferably said patient has a hsCRP level equal to or higher than about 6.2 mg/L. Preferably said patient has a hsCRP level equal to or higher than about 10 mg/L. Preferably said patient has a hsCRP level equal to or higher than about 20 mg/L. In one further embodiment, this patient is a smoker. In one further embodiment, this patient is a current smoker.

In one aspect the present invention provides an IL-1β binding antibody or a functional fragment thereof for use in the treatment and/or prevention of cancer, e.g., cancer having at least a partial inflammatory basis in a patient, wherein the efficacy of the treatment correlates with the reduction of hsCRP in said patient, comparing to prior treatment. In one embodiment, the present invention provides an IL-1β binding antibody or a functional fragment thereof for use in the treatment of cancer, e.g., cancer having at least a partial inflammatory basis, wherein hsCRP level, of said patient has reduced to below about 5.2 mg/L, preferably to below about 3.2 mg/L, preferably to below about 2.2 mg/L, about 6 months, or preferably about 3 months from the first administration of said IL-1β binding antibody or a functional fragment thereof at a proper dose, preferably according to the dosing regimen of the present invention.

In one aspect the present invention provides an IL-1β binding antibody or a functional fragment thereof (e.g., canakinumab or gevokizumab) for use in the treatment and/or prevention of cancers that have at least a partial inflammatory basis in a patient, wherein the hsCRP level of said patient has reduced by at least about 35% or at least about 50% or at least about 60% 6 months, or preferably 3 month from the first administration of said IL-1β binding antibody or a functional fragment thereof at a proper dose, preferably according to the dosing regimen of the present invention, as compared to the hsCRP level just prior to the first administration of the IL-1β binding antibody or a functional fragment thereof, canakinumab or gevokizumab). Further preferably the hsCRP level of said patient has reduced by at least about 20%, at least about 25%, at least about 25 and up to about 34%, at least about 34% and up to about 45%, at least about 20% and up to about 34%, or at least about 50% or at least about 60% after the first administration of the DRUG of the invention according to the dose regimen of the present invention.

In one aspect, the present invention provides IL-6 use as a biomarker in the treatment and/or prevention of cancer, e.g., cancer having at least a partial inflammatory basis, with an IL-1β inhibitor, e.g., IL-1β binding antibody or a functional fragment thereof. The level of IL-6 is possibly relevant in determining whether a patient with diagnosed or undiagnosed cancer or is at risk of developing cancer should be treated with an IL-1β binding antibody or a functional fragment thereof. In one embodiment, patient is eligible for the treatment and/or prevention if the level of IL-6 is equal to or higher than about 1.9 pg/ml, higher than about 2 pg/ml, higher than about 2.2 pg/ml, higher than about 2.5 pg/ml, higher than about 2.7 pg/ml, higher than about 3 pg/ml, higher than about 3.5 pg/ml, as assessed prior to the administration of the IL-1β binding antibody or a functional fragment thereof. Preferably the patient has an IL-6 level equal to or higher than about 2.5 mg/L

In one aspect the present invention provides an IL-1β binding antibody or a functional fragment thereof for use in the treatment and/or prevention of cancer, e.g., cancer having at least a partial inflammatory basis in a patient, wherein the efficacy of the treatment correlates with the reduction of IL-6 in said patient, comparing to prior treatment. In one embodiment, the present invention provides an IL-1β binding antibody or a functional fragment thereof for use in the treatment of cancer, e.g., cancer having at least a partial inflammatory basis, wherein hsCRP level, of said patient has reduced to below about 2.2 pg/ml, preferably to below about 2 pg/ml, preferably to below about 1.9 pg/ml about 6 months, or preferably about 3 months from the first administration of said IL-1β binding antibody or a functional fragment thereof at a proper dose, preferably according to the dosing regimen of the present invention.

In one aspect the present invention provides an IL-1β binding antibody or a functional fragment thereof (e.g., canakinumab or gevokizumab) for use in the treatment and/or prevention of cancers, e.g., cancers that have at least a partial inflammatory basis in a patient, wherein the IL-6 level of said patient has reduced by at least about 20%, at least about 25%, at least about 25 and up to about 34%, at least about 34% and up to about 45%, at least about 20% and up to about 34%, or at least about 50% or at least about 60% about 6 months, or preferably about 3 months from the first administration of said IL-1β binding antibody or a functional fragment thereof (e.g., canakinumab or gevokizumab) at a proper dose, preferably according to the dosing regimen of the present invention, as compared to the IL-6 level just prior to the first administration. Further preferably the IL-6 level of said patient has reduced by least about 35% or at least about 50% or at least about 60% after the first administration of the DRUG of the invention according to the dose regimen of the present invention.

The reduction of the level of hsCRP and the reduction of the level of IL-6 can be used separately or in combination to indicate the efficacy of the treatment or as prognostic markers.

### Inhibition of Angiogenesis

In one aspect, the present invention provides an IL-1β binding antibody or a functional fragment thereof, suitably canakinumab or gevokizumab, for use in a patient in need thereof in the treatment of cancer, e.g., a cancer having at least partial inflammatory basis, wherein a therapeutically amount is administered to inhibit angiogenesis in said patient. Without wishing to be bound by theory, it is hypothesized that the inhibition of IL-1β pathway can lead to inhibition or reduction of angiogenesis, which is a key event for tumor growth and for tumor metastasis. In clinical settings the inhibition or reduction of angiogenesis can be measured by tumor shrinkage, no tumor growth (stable disease), prevention of metastasis or delay of metastasis.

All the disclosed uses throughout this application, including but not limited to, doses and dosing regimens, combinations, route of administration and biomarkers can be applied to the aspect of inhibition or reduction of angiogenesis. In one embodiment, canakinumab or gevokizumab used in combination of one or more anti-cancer therapeutic agents. In one embodiment, the one or more chemotherapeutic agents is an anti-Wnt inhibitor, preferably Vantictumab. In one embodiment, the one or more therapeutic agents is a VEGF inhibitor, preferably bevacizumab or ramucirumab.

### Inhibition of Metastasis

Without wishing to be being bound by theory, it is hypothesized that the inhibition of IL-1β pathway can lead to inhibition or reduction of tumor metastasis. Until now there have been no reports on the effects of canakinumab on metastasis. Data presented in example 1 demonstrate that IL-1β activates different pro-metastatic mechanisms at the primary site compared with the metastatic site: Endogenous production of IL-1β by breast cancer cells promotes epithelial to mesenchymal transition (EMT), invasion, migration and organ specific homing. Once tumor cells arrive in the bone environment contact between tumor cells and osteoblasts or bone marrow cells increase IL-1β secretion from all three cell types. These high concentrations of IL-1β cause proliferation of the bone metastatic niche by stimulating growth of disseminated tumor cells into overt metastases. These pro-metastatic processes are inhibited by administration of anti-IL-1β treatments, such as canakinumab or gevokizumab.

Therefore, targeting IL-1β with an IL-1β binding antibody represents a novel therapeutic approach for cancer patients at risk of progressing to metastasis by preventing seeding of new metastases from established tumors and retaining tumor cells already disseminated in the bone in a state of dormancy. The models described have been designed to investigate bone metastasis and although the data show a strong link between IL-1β expression and bone homing, it does not exclude IL-1β involvement in metastasis to other sites.

Accordingly, in one aspect, the present invention provides an IL-1β binding antibody or a functional fragment thereof, suitably canakinumab or gevokizumab, for use in a patient in the treatment of cancer, e.g., a cancer having at least partial inflammatory basis, wherein a therapeutically amount is administered to inhibit metastasis in said patient.

All the disclosed uses throughout this application, including but not limited to, doses and dosing regimens, combinations, route of administration and biomarkers can be applied to the embodiment of metastasis inhibition.

### Prevention

In one aspect the present invention provides the use of an IL-1β binding antibody or a functional fragment thereof, suitably canakinumab or gevokizumab, in the prevention of cancer, e.g., cancers that have at least a partial inflammatory basis in a patient. The term “prevent”, “preventing” or “prevention” as used herein means the prevention or delay the occurrence of cancer in a subject who is otherwise at high risk of developing cancer.

Without wishing to be bound by the theory, it is hypothesized that chronic inflammation, either local or systematic, especially local inflammation, creates an immunosuppresive microenvironment that promotes tumor growth and dissemination. IL-1β binding antibody or a functional fragment thereof reduces chronic inflammation, especially IL-1β mediated chronic inflammation, and thereby prevents or delays the occurrence of cancer in a subject who has otherwise local or systematic chronic inflammation.

One way of determining local or systematic chronic inflammation is through measuring the level of C-reactive protein (hsCRP). In one embodiment, the present invention provides an IL-1β binding antibody or a functional fragment thereof, suitably canakinumab or gevokizumab, for use in the prevention of cancer, e.g., cancers that have at least a partial inflammatory basis, including lung cancer, in a subject with a high sensitive C-reactive protein (hsCRP) equal to or higher than 4.2, equal or higher than 6.5 mg/L, equal to or higher than 8.5 mg/L, or higher than 11 mg/L as assessed prior to the administration of the IL-1β binding antibody or functional fragment thereof.

In one embodiment, the present invention provides the use of an IL-1β binding antibody or a functional fragment thereof in the prevention of lung cancer, especially NSCLC in a patient, wherein said patient is a heavy smoker. The term “heavy smoker” as used here, refers to a person who smokes or who had smoked for at least 3 years in a row at least 20, or at least 30 cigarettes per day. In one embodiment, the heavy smoker is above 65 years old.

In one embodiment, the present invention provides the use of an IL-1β binding antibody or a functional fragment thereof in the prevention of cancer, e.g., cancers that have at least a partial inflammatory basis, especially lung cancer, especially NSCLC in a patient, wherein said patient has chronic lung inflammation indicated by higher than normal hsCRP level, suitably equal or higher than 6 mg/L.

In the prevention setting, it is possible that IL-1β binding antibody or a functional fragment thereof is administered as monotherapy.

In the prevention setting, it is possible that the dose of IL-1β binding antibody or a functional fragment thereof per treatment is not the same as, likely less than, that in the treatment setting. The prevention dose is likely at most half, preferably half of the treatment dose. The interval between the prevention doses is likely not the same as, likely longer than, that between the treatment doses. It is likely the interval is doubled or tripled. It is likely that the dose per treatment is the same as in the treatment settings but the dosing interval is elongated. This is preferred as longer dosing interval provides convenience and hence higher compliance. It is likely that both the dose per treatment is reduced and the dosing interval is elongated.

In one preferred embodiment, canakinumab is administered at a dose of about 100 mg to about 400 mg, preferably about 200 mg monthly, every other month or quarterly, preferably subcutaneously or preferably about 100 mg monthly, every other month or quarterly, preferably subcutaneously. In another embodiment, said IL-1β binding antibody is gevokizumab or a functional fragment thereof. In one preferred embodiment, gevokizumab is administered at a dose of about 15 mg to about 60 mg. In one preferred embodiment, gevokizumab is administered monthly, every other month or quarterly. In one preferred embodiment, gevokizumab is administered at a dose of about 15 mg monthly, every other month or quarterly. In one preferred embodiment, gevokizumab is administered at a dose of about 30 mg monthly, every other month or quarterly. In one embodiment,s gevokizumab is administered subcutaneously. In one embodiment,s gevokizumab is administered introvenously. In one embodiment, canakimab or gevokizumab is administered by an auto-injector.

In one embodiment, the risk of developing cancer, especially lung cancer, in patients receiving the prevention treatment according to the present invention is reduced by at least about 30%, preferably at least about 50%, preferably at least about 60%, preferably compared to not receiving Treatment of the Invention in the prevention settings.

Neo-Adjuvant

The term neo-adjuvant is understood as radio or chemotherapy prior to surgery. The purpose of neo-adjuvant is normally to reduce the tumor size for easy or more complete resection of the tumor.

Chronic inflammation and IL-1β have been associated with a poor histological response to neo-adjuvant therapy and risk of developing cancer (Delitto et al., BMC cancer. 2015'15:783). Without wishing to be bound by the theory, by reducing inflammation, IL-1β binding antibody or a functional fragment thereof helps improving the cancer treatment effect, especially synergizing the radiotherapy effect or chemotherapy effect in causing tumor shrinkage.

In one aspect, the present invention provides an IL-1β binding antibody or a functional fragment thereof, suitably canakinumab or gevokizumab, for use, alone or preferably in combination with radiotherapy, or in combination with one or more therapeutic agents, in the treatment of cancer prior to surgery. In one embodiment, the one or more therapeutic agents is the SoC treatment in the neo-adjuvant setting in that cancer indication. In one embodiment, the one or more therapeutic agents is a check point inhibitor, preferably selected from group consisting of nivolumab, pembrolizumab, atezolizumab, avelumab, durvalumab and spartalizumab, preferred pembrolizumab or nivolumab. In one embodiment, the one or more therapeutic agents is a chemotherapeutic agent. In one embodiment, the one or more therapeutic agents is a chemotherapeutic agent, wherein the chemotherapeutic agent is not an agent used in targeted therapy.

Neo-adjuvant treatment is normally common in treating breast cancer, gastric cancer, CRC, lung cancer, pancreatic cancer and prostate cancer, preferably those cancers are resectable cancers.

Major pathological response (MPR), defined as ≤10% residual viable tumor, was demonstrated to positively correlate to disease free survival (DFS) and overall survival (OS) (Pataer et al 2012; Hellmann et al 2014) and thus is considered as a surrogate efficacy endpoint for neo-adjuvant studies. In one embodiment, patient has at least 10%, at least 20%, at least 30%, at least 40% chance of having MPR after having completed the neo-adjuvant treatment.

In one embodiment, patient is treated with 2 cycles of canakinumab, 3 weeks or 4 weeks for each cycle. In one embodiment, patient is treated with 2 cycles of gevokizumab, 3 weeks or 4 weeks for each cycle. In one embodiment, the one or more therapeutic agents is pembrolizumab. In one embodiment, the one or more therapeutic agents is nivolumab.

In one embodiment, the cancer is NSCLC, suitably Stage I-IIIA resectabal NSCLC, wherien patient is treatment naive. In one embodiment, DRUG of the invention is canakinumab or gevolizumab, to be used alone or in combination with one or more therapeutical agents in the neo-adjuvant treatment of NSCLC. In one embodiment, the one or more therapeutical agents is platinum-based chemotherapy (cisplatin or carboplatin, combined with other agents). In one embodiment, the one or more therapeutical agents is a check point inhibitor, preferably pembrolizumab. In one embodiment, about 200 mg canakinumab is administered for 2 cycles, 3 week for one cycle, alone or in combination with pembrolizumab, preferably about 200 mg. In one embodiment, patient has at least 10%, at least 20%, at least 30%, at least 40% chance of having MPR after having completed the neo-adjuvant treatment.

### Adjuvant Treatment

In one aspect, the present invention provides an IL-1β binding antibody or a functional fragment thereof, suitably canakinumab or gevokizumab, for use, alone or in combination with one or more therapeutic agents, in the prevention of recurrence or relapse of cancer, e.g., cancer having at least a partial inflammatory basis, which has been surgically removed (resected “adjuvant chemotherapy”).

Without wishing to be bound by the theory, after tumor has been surgically removed, it is possible that the inflammation is greatly reduced due to surgery. The IL-1β or the hsCRP level is no longer higher than normal. It is however reasonable to expect that the DRUG of the invention can prevent or delay the recurrence or relapse of cancer by keeping inflammation under control and thereby preventing the re-formation of IL-1β mediated immune suppressive tumormicroenvironment that promote tumor growth and metastasis. Furthermore after tumor has been surgically removed, the patient's immune system can regain its surveillance function in eliminating remaining tumor loci/cells. By reducing inflammation, IL-1β binding antibody or a functional fragment thereof helps maintaining or improving the surveillance function of the immune system and thereby prevents or delays tumor recurrence or relapse of cancer.

In one embodiment, the one or more therapeutic agent is the standard of care adjuvant (other than the treatment of DRUG of the invention) treatment in that cancer indication. Standard of Care (SoC) adjuvant treatment varies depending on the cancer. Suitably the SoC adjuvant treatment is a chemotherapy, a radiotherapy, a targeted therapy or a checkpoint inhibitor therapy. Often SoC drug in the adjuvant treatment is the same drug as SoC in the first line treatment, only that in adjuvant setting the drug is administered for a short period, normally not longer than 6 months for chemotherapies. Normally not longer than 12 months for check point inhibitors. For example in NSCLC, SoC adjuvant treatment is cisplatin-based doublet chemotherapy, normally taking for 4 cycles. For example in RCC, the SoC adjuvant treatment is pembrolizumab for one year.

In one embodiment, DRUG of the invention is administered after the patient has completed the SoC adjuvant treatment, suitably chemotherapy or radiotherapy, suitably as single agent.

In one embodiment, IL-1β binding antibody or a functional fragment thereof, suitably canakinumab or gevokizumab, is added on top of the SoC adjuvant treatment, preferably administered at the beginning of the patient's SoC adjuvant treatment. In one embodiment, the SoC adjuvant treatment is a targeted therapy or a immunotherapy. Suitably the combination treatment last for 6 months to one year.

In one embodiment, the patient receives DRUG of the invention, suitably canakinumab or gevokizumab, for at least 6 months, preferably for at least 12 months, preferably for 12 months. Due to the good safety profile it is possible that DRUG of the invention, suitably canakinumab or gevokizumabm is administered longer than one year, for example for 2 years, for 3 years or for 5 years or till the recurrence or relapse of cancer, either in combination with SoC adjuvant treatment or preferably as a single agent.

In one embodiment, DRUG of the invention, suitably canakinumab or gevokizumab, is the sole post-surgery adjuvant treatment, in a patient who does not receive other adjuvant treatment or could not have completed the SoC adjuvant treatment. Chemotherapy or check point inhibitors results in many undesired side effects. Thus the present invention provides an alternative post-surgery adjuvant treatment, preferably with very low or much better tolerated side effects.

In the adjuvant settings, DRUG of the invention, suitably canakinumab or gevokizumab, is administered according to the dosing regiment of the present invention. When used as monotherapy, the dosing interval can be flexible. For example, canakinumab or gevokizumab can be administered in the loading phase and in the maintenance phase, wherein a lower amount of drug is given during the maintenance phase. For example canakinumab or gevokizumab can be administered every 3 weeks or monthly post surgery in the loading phase. The dose interval can be doubled or tripled in the maintenance phase. In one embodiment, the loading phase is at least 6 months, preferably at least 12 months, preferably 12 months. In one embodiment, the maintenance dose is at least 12 months or at least 24 months, or till the recurrence or relapse of the cancer.

In one embodiment, the present invention provides an IL-1β binding antibody or a functional fragment thereof, suitably canakinumab or gevokizumab, for use in the prevention of recurrence or relapse of cancer, e.g., cancer having at least a partial inflammatory basis, which has been surgically removed (resected “adjuvant chemotherapy”), wherein the disease free survival (DFS) in patients receiving Treatment of the Invention is at least 6 months or at least 9 months, or at least 12 months longer than not receiving Treatment of the Invention in the adjuvant setting. DFS is defined as the time from the date of randomization to the date of detection of first disease recurrence. In one embodiment, patient is followed up every 12 weeks after the completion of the adjuvant treatment of the present invention. In one embodiment, detection of first disease recurrence will be done by clinical evaluation that includes physical examination, and radiological tumor measurements as determined by the investigator. In one embodiment, patient not receiving Treatment of the Invention did not receive any treatment. In one embodiment, patient not receiving Treatment of the Invention received considered SoC treatment at the time of trial for the tested cancer indication.

Normally after resection of cancer, patient is in the disease free status (DFS), which will end at the time of cancer progression or recurrence. In one embodiment, the hazard ratio (HR) of the patient in losing the DFS status is reduced by at least about 20%, at least about 30%, by upto about 50%, by upto about 70%, or by about 20% to about 30%, by about 30% to about 40%, compare to not receiving Treatment of the Invention.

In one embodiment, the DFS of the patient receiving Treatment of the Invention is at least 24 months, preferably at least 48 months.

In the adjuvant setting patients are considered healthy. To improve patients convenience and quality of life, canakinumab or gevokizumab is administered subcutaneously, by a prefilled syringe or preferably by an auto-injector, preferably at patients' home.

### First Line Treatment

In one embodiment, the present invention provides an IL-1β antibody or a functional fragment thereof, suitably canakinumab or gevokizumab, for use as the first line treatment of cancer, e.g., cancer having at least a partial inflammatory basis. The term “first line treatment” means said patient is given the IL-1β antibody or a functional fragment thereof before the patient develops resistance to the initial treatment with one or more other therapeutic agents. Preferably one or more other therapeutic agents is a platinum-based mono or combination therapy, a targeted therapy, such a tyrosine inhibitor therapy, a checkpoint inhibitor therapy or any combination thereof. As first line treatment, the IL-1β antibody or a functional fragment thereof, such as canakinumab or gevokizumab, can be administered to patient as monotherapy or preferably in combination with one or more therapeutic agents, such as a check point inhibitor, particularly a PD-1 or PD-L1 inhibitor, preferably pembrolizumab, with or without one or more small molecule chemotherapeutic agent. In one embodiment, as first line treatment, the IL-1β antibody or a functional fragment thereof, such as canakinumab or gevokizumab, can be administered to patient in combination with the standard of care therapy for that cancer. Preferably canakinumab or gevokizumab is administered as the first line treatment until disease progression.

### Second Line Treatment

In one embodiment, the present invention provides an IL-1β antibody or a functional fragment thereof, suitably canakinumab or gevokizumab, for use as the second or third line treatment of cancer, e.g., cancer having at least a partial inflammatory basis. The term “the second or third line treatment” means IL-1β antibody or a functional fragment thereof is administered to a patient with cancer progression on or after one or more other therapeutic agent treatment, especially disease progression on or after FDA-approved first line therapy for that cancer. Preferably one or more other therapeutic agent is a chemotherapeutic agent, such as platinum-based mono or combination therapy, a targeted therapy, such a tyrosine inhibitor therapy, a checkpoint inhibitor therapy or any combination thereof. As the second or third line treatment, the IL-13 antibody or a functional fragment thereof can be administered to the patient as monotherapy or preferably in combination with one or more therapeutic agent, including the continuation of the early treatment with the same one or more therapeutic agent. Preferably canakinumab or gevokizumab is administered as the 2^{nd}/3^{rd} line treatment until disease progression.

### Continuous Treatment

In one aspect the present invention also provides an IL-1β binding antibody or a functional fragment thereof, suitably gevokizumab or canakinumab, for use in the treatment of cancer, e.g., cancer having at least partial inflammatory basis, wherein IL-1β binding antibody or a functional fragment thereof is administered to a patient in more than one line of treatment.

Without wishing to be bound by the theory, it is hypothesized that, unlike chemotherapeutic agents or targeted therapy, which have a direct killing or inhibiting effect on the cancer cells and thereby selecting resist cells, DRUG of the invention works on the tumor-microenvironment and does not seem to lead to drug resistance. Furthermore unlike chemotherapeutic agents or check point inhibitors, IL-1β binding antibody or a functional fragment thereof, such as gevokizumab or canakinumab, has much less undesired side effects. Patients unlikely develop intolerance and therefore can continue receive DRUG of the invention and continue the benefit of elimination or reduction of IL-1β mediated inflammation in the course of cancer treatment.

In one embodiment, DRUG of the invention, suitably canakinumab or gevokizumab, can be used in 2, 3 or all lines of the treatment of cancer in the same patient. Treatment line typically includes but not limited to neo-adjuvant treatment, adjuvant treatment, first line treatment, 2^{nd} line treatment, 3^{rd} line treatment and further line of treatment. Patient normally changes lines of treatment after surgery, after disease progression or after developing drug resistance to the current treatment. In one embodiment, DRUG of the invention is continued after patient develops resistant to the current treatment. In one embodiment, DRUG of the invention is continued to the next line of treatment. In one embodiment, DRUG of the invention is continued after disease progression. In one embodiment, DRUG of the invention is continued until death or until palliative care.

In one embodiment, the present invention provides DRUG of the invention, suitably canakinumab or gevokizumab, for use in re-treating cancer in a patient, wherein the patient was treated with the same DRUG of the invention in the previous treatment. In one embodiment, the previous treatment is the neo-adjuvant treatment. In one embodiment, the previous treatment is the adjuvant treatment. In one embodiment, the previous treatment is the first line treatment. In one embodiment, the previous treatment is the second line treatment.

In one embodiment, the cancer is lung cancer, especially NSCLC, the IL-1β binding antibody is canakinumab, wherein canakinumab is administered to the patient, wherein the patient was treated with canakinumab in the previous treatment. In one embodiment, the previous treatment is the neo-adjuvant treatment. In one embodiment, the previous treatment is the adjuvant treatment. In one further embodiment the adjuvant treatment is for patients with stage II to IIIA and IIIB (T>5 cm N2) non-small cell lung cancer following complete surgical resection. In one embodiment, the previous treatment is the first line treatment. In one further embodiment the first line treatment is canakinumab in combination with pembrolizumab and platinum based chemotherapy, for the treatment of patients with locally advanced or metastatic non-small cell lung cancer. In one embodiment, the previous treatment is the second line treatment. In one further embodiment the second line treatment is canakinumab in combination with docetaxel for the treatment of patients with locally advanced or metastatic non-small cell lung cancer previously treated with PD-(L)1 inhibitors and platinum-based chemotherapy, with or without canakinumab.

### Combination

In one aspect, the present invention provides an IL-1β binding antibody or a functional fragment thereof, suitably canakinumab or gevokizumab, for use in a patient in need thereof in the treatment of a cancer, particularly cancer having at least partial inflammatory basis, in combination with a radiotherapy, in combination with a cell-based therapy, or in combination with one or more therapeutic agents, e.g., chemotherapeutic agents or e.g., a check point inhibitor, or in combination with both radiotherapy and one or more therapeutic agents.

Without wishing to be bound by the theory, it is believed that typical cancer development requires two steps. Firstly, gene alteration results in cell growth and proliferation no longer subject to regulation. Secondly, the abnormal tumor cells evade surveillance of the immune system. Inflammation plays important role in the second step. Therefore, control of inflammation, can stop cancer development at the early or earlier stage. Thus it is expected that blocking IL-1β pathway to reduce inflammation would have a general benefit, particularly improvement of the treatment efficacy on top of the standard of care, which is normally mainly to directly inhibit the growth and proliferation of the malignant cells. In one embodiment, the one or more therapeutic agents, e.g., chemotherapeutic agents is the standard of care agents of said cancer, particularly cancer having at least partial inflammatory basis.

Check point inhibitors de-suppress the immune system through a mechanism different from IL-1β inhibitors. Thus the addition of IL-1β inhibitors, particularly IL-1β binding antibodies or a functional fragment thereof to the standard Check point inhibitors therapy will further active the immune response, particularly at the tumor microenvironment.

In one embodiment, the one or more therapeutic agents is nivolumab.

In one embodiment, the one or more therapeutic agents is pembrolizumab.

In one embodiment, the one or more therapeutic agents is spartalizumab (PDR001).

In one embodiment, the one or more therapeutic agent, e.g., chemotherapeutic agents is nivolumab and ipilimumab.

In one embodiment, the one or more chemotherapeutic agents is cabozantinib, or a pharmaceutically acceptable salt thereof.

In one embodiment, the or more therapeutic agent, e.g., chemotherapeutic agent is Atezolizumab plus bevacizumab.

In one embodiment, the one or more chemotherapeutic agents is bevacizumab.

In one embodiment, the one or more chemotherapeutic agents is FOLFIRI, FOLFOX or XELOX.

In one embodiment, the one or more chemotherapeutic agent is FOLFIRI plus bevacizumab or FOLFOX plus bevacizumab.

In one embodiment, the one or more chemotherapeutic agent is platinum-based doublet chemotherapy (PT-DC).

In one embodiment, the one or more chemotherapeutic agent is MBG453.

In one embodiment, the one or more chemotherapeutic agent is NIS793.

Therapeutic agents are cytotoxic and/or cytostatic drugs (drugs that kill malignant cells, or inhibit their proliferation, respectively) as well as check point inhibitors. Chemotherapeutic agents can be, for example, small molecule agents, biologics agents (e.g., antibodies, cell and gene therapies, cancer vaccines), hormones or other natural or synthetic peptide or polypeptides. Commonly known chemotherapeutic agent includes, but is not limited to, platinum agents (e.g., cisplatin, carboplatin, oxaliplatin, nedaplatin, triplatin, lipoplatin, satraplatin, picoplatin), antimetabolites (e.g., methotrexate, 5-Fluorouracil, gemcitabine, pemetrexed, edatrexate), mitotic inhibitors (e.g., paclitaxel, albumin-bound paclitaxel, docetaxel, taxotere, docecad), alkylating agents (e.g., cyclophosphamide, mechlorethamine hydrochloride, ifosfamide, melphalan, thiotepa), vinca alkaloids (e.g., vinblastine, vincristine, vindesine, vinorelbine), topoisomerase inhibitors (e.g., etoposide, teniposide, topotecan, irinotecan, camptothecin, doxorubicin), antitumor antibiotics (e.g., mitomycin C) and/or hormone-modulating agents (e.g., anastrozole, tamoxifen). Examples of anti-cancer agents used for chemotherapy include Cyclophosphamide (Cytoxan®), Methotrexate, 5-Fluorouracil (5-FU), Doxorubicin (Adriamycin®), Prednisone, Tamoxifen (Nolvadex®), Paclitaxel (Taxol®), Albumin-bound paclitaxel (nab-paclitaxel, Abraxane®), Leucovorin, Thiotepa (Thioplex®), Anastrozole (Arimidex®), Docetaxel (Taxotere®), Vinorelbine (Navelbine®), Gemcitabine (Gemzar®), Ifosfamide (Ifex®), Pemetrexed (Alimta®), Topotecan, Melphalan (L-Pam®), Cisplatin (Cisplatinum®, Platinol®), Carboplatin (Paraplatin®), Oxaliplatin (Eloxatin®), Nedaplatin (Aqupla®), Triplatin, Lipoplatin (Nanoplatin®), Satraplatin, Picoplatin, Carmustine (BCNU; BiCNU®), Methotrexate (Folex®, Mexate®), Edatrexate, Mitomycin C (Mutamycin®), Mitoxantrone (Novantrone®), Vincristine (Oncovin®), Vinblastine (Velban®), Vinorelbine (Navelbine®), Vindesine (Eldisine®), Fenretinide, Topotecan, Irinotecan (Camptosar®), 9-amino-camptothecin [9-AC], Biantrazole, Losoxantrone, Etoposide, and Teniposide.

In one embodiment, the preferred combination partner for the IL-1β binding antibody or a functional fragment thereof (e.g., canakinumab or gevokizumab) is a mitotic inhibitor, preferably docetaxel. In one embodiment, the preferred combination partner for canakinumab is a mitotic inhibitor, preferably docetaxel. In one embodiment, the preferred combination partner for gevokizumab is a mitotic inhibitor, preferably docetaxel. In one embodiment, said combination is used for the treatment of lung cancer, especially NSCLC.

In one embodiment, the preferred combination partner for the IL-1β binding antibody or a functional fragment thereof (e.g., canakinumab or gevokizumab) is a platinum agent, preferably cisplatin. In one embodiment, the preferred combination partner for canakinumab is a platinum agent, preferably cisplatin. In one embodiment, the preferred combination partner for gevokizumab is a platinum agent, preferably cisplatin. In one embodiment, the one or more chemotherapeutic agent is a platinum-based doublet chemotherapy (PT-DC).

Chemotherapy may comprise the administration of a single anti-cancer agent (drug) or the administration of a combination of anti-cancer agents (drugs), for example, one of the following, commonly administered combinations of: carboplatin and taxol; gemcitabine and cisplatin; gemcitabine and vinorelbine; gemcitabine and paclitaxel; cisplatin and vinorelbine; cisplatin and gemcitabine; cisplatin and paclitaxel (Taxol); cisplatin and docetaxel (Taxotere); cisplatin and etoposide; cisplatin and pemetrexed; carboplatin and vinorelbine; carboplatin and gemcitabine; carboplatin and paclitaxel (Taxol); carboplatin and docetaxel (Taxotere); carboplatin and etoposide; carboplatin and pemetrexed. In one embodiment, the one or more chemotherapeutic agent is a platinum-based doublet chemotherapy (PT-DC).

Another class of chemotherapeutic agents are the inhibitors, especially tyrosine kinase inhibitors, that specifically target growth promoting receptors, especially VEGF-R, EGFR, PFGF-R and ALK, or their downstream members of the signalling transduction pathway, the mutation or overproduction of which results in or contributes to the oncogenesis of the tumor at the site (targeted therapies). Exemplary of targeted therapies drugs approved by the Food and Drug administration (FDA) for the targeted treatment of lung cancer include but not limited bevacizumab (Avastin®), crizotinib (Xalkori®), erlotinib (Tarceva®), gefitinib (Iressa®), afatinib dimaleate (Gilotrif®), ceritinib (LDK378/Zykadia™), everolimus (Afinitor®), ramucirumab (Cyramza®), osimertinib (Tagrisso™), necitumumab (Portrazza™), alectinib (Alecensa®), atezolizumab (Tecentriq™), brigatinib (Alunbrig™), trametinib (Mekinist®), dabrafenib (Tafinlar®), sunitinib (Sutent®) and cetuximab (Erbitux®).

In one embodiment, the one or more chemotherapeutic agent to be combined with the IL-1β binding antibody or fragment thereof, suitably canakinumab or gevokizumab, is the agent that is the standard of care agent for lung cancer, including NSCLC and SCLC. Standard of care, can be found, for example from American Society of Clinical Oncology (ASCO) guideline on the systemic treatment of patients with stage IV non-small-cell lung cancer (NSCLC) or American Society of Clinical Oncology (ASCO) guideline on Adjuvant Chemotherapy and Adjuvant Radiation Therapy for Stages I-IIIA Resectable Non-Small Cell Lung Cancer.

In one embodiment, the one or more chemotherapeutic agent to be combined with the IL-1β binding antibody or fragment thereof, suitably canakinumab or gevokizumab, is a platinum containing agent or a platinum-based doublet chemotherapy (PT-DC). In one embodiment, said combination is used for the treatment of lung cancer, especially NSCLC. In one embodiment, one or more chemotherapeutic agent is a tyrosine kinase inhibitor. In one preferred embodiment said tyrosine kinase inhibitor is a VEGF pathway inhibitor or an EGF pathway inhibitor. In one embodiment, the one or more chemotherapeutic agent is check-point inhibitor, preferably pembrolizumab. In one embodiment, said combination is used for the treatment of lung cancer, especially NSCLC.

In one embodiment, the one or more therapeutic agent to be combined with the IL-1 binding antibody or fragment thereof, suitably canakinumab or gevokizumab, is a check-point inhibitor. In one further embodiment, said check-point inhibitor is nivolumab. In one embodiment, said check-point inhibitor is pembrolizumab. In one further embodiment, said check-point inhibitor is atezolizumab. In one further embodiment, said check-point inhibitor is PDR-001 (spartalizumab). In one embodiment, said check-point inhibitor is durvalumab. In one embodiment, said check-point inhibitor is avelumab. Immunotherapies that target immune checkpoints, also known as checkpoint inhibitors, are currently emerging as key agents in cancer therapy. The immune checkpoint inhibitor can be an inhibitor of the receptor or an inhibitor of the ligand. Examples of the inhibiting targets include but not limited to a co-inhibitory molecule (e.g., a PD-1 inhibitor (e.g., an anti-PD-1 antibody molecule), a PD-L1 inhibitor (e.g., an anti-PD-L1 antibody molecule), a PD-L2 inhibitor (e.g., an anti-PD-L2 antibody molecule), a LAG-3 inhibitor (e.g., an anti-LAG-3 antibody molecule), a TIM-3 inhibitor (e.g., an anti-TIM-3 antibody molecule), an activator of a co-stimulatory molecule (e.g., a GITR agonist (e.g., an anti-GITR antibody molecule), a cytokine (e.g., IL-15 complexed with a soluble form of IL-15 receptor alpha (IL-15Ra), an inhibitor of cytotoxic T-lymphocyte-associated protein 4 (e.g., an anti-CTLA-4 antibody molecule) or any combination thereof.

### PD-1 Inhibitors

In one aspect of the invention, the IL-1β inhibitor or a functional fragment thereof is administered together with a PD-1 inhibitor. In one some embodiment the PD-1 inhibitor is chosen from PDR001 (spartalizumab) (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHRI210 (Incyte), or AMP-224 (Amplimmune).

In one embodiment, the PD-1 inhibitor is an anti-PD-1 antibody. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

TABLE A
Amino acid and nucleotide sequences of exemplary
anti-PD-1 antibody molecules

| | | |
|---|---|---|
| BAP049-Clone-B HC | | |
| SEQ ID NO: 506 | VH | EVQLVQSGAEVKKPGESLRISCKGSGYTFTTYWMHWVRQAT |
| | | GQGLEWMGNIYPGTGGSNFDEKFKNRVTITADKSTSTAYMEL |
| | | SSLRSEDTAVYYCTRWTTGTGAYWGQGTTVTVSS |
| BAP049-Clone-B LC | | |
| SEQ ID NO: 516 | VL | EIVLTQSPATLSLSPGERATLSCKSSQSLLDSGNQKNFLTWYQQ |
| | | KPGKAPKLLIYWASTRESGVPSRFSGSGSGTDFTFTISSLQPEDI |
| | | ATYYCQNDYSYPYTFGQGTKVEIK |
| BAP049-Clone-E HC | | |
| SEQ ID NO: 506 | VH | EVQLVQSGAEVKKPGESLRISCKGSGYTFTTYWMHWVRQAT |
| | | GQGLEWMGNIYPGTGGSNFDEKFKNRVTITADKSTSTAYMEL |
| | | SSLRSEDTAVYYCTRWTTGTGAYWGQGTTVTVSS |
| BAP049-Clone-E LC | | |
| SEQ ID NO: 520 | VL | EIVLTQSPATLSLSPGERATLSCKSSQSLLDSGNQKNFLTWYQQ |
| | | KPGQAPRLLIYWASTRESGVPSRFSGSGSGTDFTFTISSLEAEDA |
| | | ATYYCQNDYSYPYTFGQGTKVEIK |

In one embodiment, the anti-PD-1 antibody is spartalizumab.

In one embodiment, the anti-PD-1 antibody is Nivolumab.

In one embodiment, the anti-PD-1 antibody molecule is Pembrolizumab.

In one embodiment, the anti-PD-1 antibody molecule is Pidilizumab.

In one embodiment, the anti-PD-1 antibody molecule is MED10680 (Medimmune), also known as AMP-514. MEDI0680 and other anti-PD-1 antibodies are disclosed in U.S. Pat. No. 9,205,148 and WO 2012/145493, incorporated by reference in their entirety. Other exemplary anti-PD-1 molecules include REGN2810 (Regeneron), PF-06801591 (Pfizer), BGB-A317/BGB-108 (Beigene), INCSHRI210 (Incyte) and TSR-042 (Tesaro).

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

### PD-L1 Inhibitors

In one aspect of the invention, the IL-1β inhibitor or a functional fragment thereof is administered together with a PD-L1 inhibitor. In some embodiments, the PD-L1 inhibitor is chosen from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises a VH comprising the amino acid sequence of SEQ ID NO: 606 and a VL comprising the amino acid sequence of SEQ ID NO: 616. In one embodiment, the anti-PD-L1 antibody molecule comprises a VH comprising the amino acid sequence of SEQ ID NO: 620 and a VL comprising the amino acid sequence of SEQ ID NO: 624.

TABLE B
Amino acid and nucleotide sequences of exemplary
anti-PD-L1 antibody molecules

| | | |
|---|---|---|
| BAP058-Clone O HC | | |
| SEQ ID NO: 606 | VH | EVQLVQSGAEVKKPGATVKISCKVSGYTFTSYWMYWVRQA |
| | | RGQRLEWIGRIDPNSGSTKYNEKFKNRFTISRDNSKNTLYLQ |
| | | MNSLRAEDTAVYYCARDYRKGLYAMDYWGQGTTVTVSS |
| BAP058-Clone O LC | | |
| SEQ ID NO: 616 | VL | AIQLTQSPSSLSASVGDRVTITCKASQDVGTAVAWYLQKPGQ |
| | | SPQLLIYWASTRHTGVPSRFSGSGSGTDFTFTISSLEAEDAATY |
| | | YCQQYNSYPLTFGQGTKVEIK |
| BAP058-Clone N HC | | |
| SEQ ID NO: 620 | VH | EVQLVQSGAEVKKPGATVKISCKVSGYTFTSYWMYWVRQA |
| | | TGQGLEWMGRIDPNSGSTKYNEKFKNRVTTTADKSTSTAYME |
| | | LSSLRSEDTAVYYCARDYRKGLYAMDYWGQGTTVTVSS |
| BAP058-Clone N LC | | |
| SEQ ID NO: 624 | VL | DVVMTQSPLSLPVTLGQPASISCKASQDVGTAVAWYQQKPG |
| | | QAPRLLIYWASTRHTGVPSRFSGSGSGTEFTLTISSLQPDDFAT |
| | | YYCQQYNSYPLTFGQGTKVEIK |

In one embodiment, the anti-PD-L1 antibody molecule is Atezolizumab (Genentech/Roche), also known as MPDL3280A, RG7446, RO5541267, YW243.55.S70, or TECENTRIQ™. Atezolizumab and other anti-PD-L1 antibodies are disclosed in U.S. Pat. No. 8,217,149, incorporated by reference in its entirety.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety.

In one embodiment, the anti-PD-L1 antibody molecule is Durvalumab (MedImmune/AstraZeneca), also known as MED14736. Durvalumab and other anti-PD-L1 antibodies are disclosed in U.S. Pat. No. 8,779,108, incorporated by reference in its entirety.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

### LAG-3 Inhibitors

In one aspect of the invention, the IL-1β inhibitor or a functional fragment thereof is administered together with a LAG-3 inhibitor. In some embodiments, the LAG-3 inhibitor is chosen from LAG525 (Novartis), BMS-986016 (Bristol-Myers Squibb), TSR-033 (Tesaro), IMP731 or GSK2831781 and IMP761 (Prima BioMed).

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

TABLE C
Amino acid and nucleotide sequences of exemplary
anti-LAG-3 antibody molecules

| | | |
|---|---|---|
| BAP050-Clone I HC | | |
| SEQ ID NO: 706 | VH | QVQLVQSGAEVKKPGASVKVSCKASGFTLTNYGMNWVRQAR |
| | | GQRLEWIGWINTDTGEPTYADDFKGRFVFSLDTSVSTAYLQISS |
| | | LKAEDTAVYYCARNPPYYYGTNNAEAMDYWGQGTTVTVSS |
| BAP050-Clone I LC | | |
| SEQ ID NO: 718 | VL | DIQMTQSPSSLSASVGDRVTITCSSSQDISNYLNWYLQKPGQSP |
| | | QLLIYYTSTLHLGVPSRFSGSGSGTEFTLTISSLQPDDFATYYCQ |
| | | QYYNLPWTFGQGTKVEIK |
| BAP050-Clone J HC | | |
| SEQ ID NO: 724 | VH | QVQLVQSGAEVKKPGASVKVSCKASGFTLTNYGMNWVRQAP |
| | | GQGLEWMGWINTDTGEPTYADDFKGRFVFSLDTSVSTAYLQI |
| | | SSLKAEDTAVYYCARNPPYYYGTNNAEAMDYWGQGTTVTVS |
| | | S |
| BAP050-Clone J LC | | |
| SEQ ID NO: 730 | VL | DIQMTQSPSSLSASVGDRVTITCSSSQDISNYLNWYQQKPGKAP |
| | | KLLIYYTSTLHLGIPPRFSGSGYGTDFTLTINNIESEDAAYYFCQ |
| | | QYYNLPWTFGQGTKVEIK |

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table D.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table D.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE D
Amino acid sequences of exemplary anti-LAG-3
antibody molecules

| | | |
|---|---|---|
| BMS-986016 | | |
| SEQ ID NO: 762 | Heavy chain | QVQLQQWGAGLLKPSETLSLTCAVYGGSFSDYYWNWIRQPPGKG |
| | | LEWIGEINHRGSTNSNPSLKSRVTLSLDTSKNQFSLKLRSVTAADTA |
| | | VYYCAFGYSDYEYNWFDPWGQGTLVTVSSASTKGPSVFPLAPCSR |
| | | STSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLGK |
| SEQ ID NO: 763 | Light chain | EIVLTQSPATLSLSPGERATLSCRASQSISSYLAWYQQKPGQAPRLL |
| | | IYDASNRATGIPARFSGSGSGTDFTLTISSLEPEDFAVYYCQQRSNW |
| | | PLTFGQGTNLEIKRTVAAPSVFIFPPSDEQLKSGTASVVCLLNNFYP |
| | | REAKVQWKVDNALQSGNSQESVTEQDSKDSTYSLSSTLTLSKADY |
| | | EKHKVYACEVTHQGLSSPVTKSFNRGEC |
| IMP731 | | |
| SEQ ID NO: 764 | Heavy chain | QVQLKESGPGLVAPSQSLSITCTVSGFSLTAYGVNWVRQPPGKGLE |
| | | WLGMIWDDGSTDYNSALKSRLSISKDNSKSQVFLKMNSLQTDDTA |
| | | RYYCAREGDVAFDYWGQGTTLTVSSASTKGPSVFPLAPSSKSTSG |
| | | GTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKE |
| | | YKCKVSNKALPAPIEKTISKAKGQPREPQVYTLPPSRDELTKNQVS |
| | | vLTCLVKGFYPSDIAVEWESNGQPENNYKTTPPVLDSDGSFFLYSKL |
| | | TVDKSRWQQGNVFSCSVMHEALHNHYTQKSLSLSPGK |
| SEQ ID NO: 765 | Light chain | DIVMTQSPSSLAVSVGQKVTMSCKSSQSLLNGSNQKNYLAWYQQ |
| | | KPGQSPKLLVYFASTRDSGVPDRFIGSGSGTDFTLTISSVQAEDLAD |
| | | YFCLQHFGTPPTFGGGTKLEIKRTVAAPSVFIFPPSDEQLKSGTASV |
| | | VCLLNNFYPREAKVQWKVDNALQSGNSQESVTEQDSKDSTYSLSS |
| | | TLTLSKADYEKHKVYACEVTHQGLSSPVTKSFNRGEC |

### TIM-3 Inhibitors

In one aspect of the invention, the IL-1β inhibitor or a functional fragment thereof is administered together with a TIM-3 inhibitor. In some embodiments, the TIM-3 inhibitor is MBG453 (Novartis) or TSR-022 (Tesaro). Historically MBG453 is often misspelt as MGB453.

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof.” incorporated by reference in its entirety.

In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment the anti-TIM-3 antibody is MBG453 comprising a VH comprising the amino acid sequence of SEQ ID NO: 806 and a VL comprising the amino acid sequence of SEQ ID NO: 816.

The antibody molecules described herein can be made by vectors, host cells, and methods described in US 2015/0218274, incorporated by reference in its entirety.

TABLE E
Amino acid and nucleotide sequences of exemplary
anti-TIM-3 antibody molecules

| | | |
|---|---|---|
| ABTIM3-hum11 | | |
| SEQ ID NO: 806 | VH | QVQLVQSGAEVKKPGSSVKVSCKASGYTFTSYNMHWVRQA |
| | | PGQGLEWMGDIYPGNGDTSYNQKFKGRVTITADKSTSTVY |
| | | MELSSLRSEDTAVYYCARVGGAFPMDYWGQGTTVTVSS |
| SEQ ID NO: 816 | VL | AIQLTQSPSSLSASVGDRVTITCRASESVEYYGTSLMQWYQQ |
| | | KPGKAPKLLIYAASNVESGVPSRFSGSGSGTDFTLTISSLQPE |
| | | DFATYFCQQSRKDPSTFGGGTKVEIK |
| ABTIM3-hum03 | | |
| SEQ ID NO: 822 | VH | QVQLVQSGAEVKKPGASVKVSCKASGYTFTSYNMHWVRQ |
| | | APGQGLEWIGDIYPGQGDTSYNQKFKGRATMTADKSTSTVY |
| | | MELSSLRSEDTAVYYCARVGGAFPMDYWGQGTLVTVSS |
| SEQ ID NO: 826 | VL | DIVLTQSPDSLAVSLGERATINCRASESVEYYGTSLMQWYQ |
| | | QKPGQPPKLLIYAASNVESGVPDRFSGSGSGTDFTLTISSLQA |
| | | EDVAVYYCQQSRKDPSTFGGGTKVEIK |

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table F. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE F
Amino acid sequences of exemplary anti-TIM-3
antibody molecules

| | | |
|---|---|---|
| APE5137 | | |
| SEQ ID NO: 830 | VH | EVQLLESGGGLVQPGGSLRLSCAAASGFTFSSYDMSWVRQAPGKGLDW |
| | | VSTISGGGTYTYYQDSVKGRFTISRDNSKNTLYLQMNSLRAEDTAVYYC |
| | | ASMDYWGQGTTVTVSSA |
| SEQ ID NO: 831 | VL | DIQMTQSPSSLSASVGDRVTITCRASQSIRRYLNWYHQKPGKAPKLLIYG |
| | | ASTLQSGVPSRFSGSGSGTDFTLTISSLQPEDFAVYYCQQSHSAPLTFGG |
| | | GTKVEIKR |
| APE5121 | | |
| SEQ ID NO: 832 | VH | EVQVLESGGGLVQPGGSLRLYCVASGFTFSGSYAMSWVRQAPGKGLE |
| | | WVSAISGSGGSTYYADSVKGRFTISRDNSKNTLYLQMNSLRAEDTAVY |
| | | YCAKKYYVGPADYWGQGTLVTVSSG |
| SEQ ID NO: 833 | VL | DIVMTQSPDSLAVSLGERATINCKSSQSVLYSSNNKNYLAWYQHKPGQP |
| | | PKLLIYWASTRESGVPDRFSGSGSGTDFTLTISSLQAEDVAVYYCQQYYS |
| | | SPLTFGGGTKIEVK |

### GITR Agonists

In one aspect of the invention, the IL-1β inhibitor or a functional fragment thereof is administered together with a GITR agonist. In some embodiments, the GITR agonist is GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises a VH comprising the amino acid sequence of SEQ ID NO: 901 and a VL comprising the amino acid sequence of SEQ ID NO: 902.

TABLE G
Amino acid and nucleotide sequences of exemplary
anti-GITR antibody molecule

| | | |
|---|---|---|
| MAB7 | | |
| SEQ ID NO: 901 | VH | EVQLVESGGGLVQSGGSLRLSCAASGFSLSSYGVDWVRQA |
| | | PGKGLEWVGVIWGGGGTYYASSLMGRFTISRDNSKNTLYL |
| | | QMNSLRAEDTAVYYCARHAYGHDGGFAMDYWGQGTLVT |
| | | VSS |
| SEQ ID NO: 902 | VL | EIVMTQSPATLSVSPGERATLSCRASESVSSNVAWYQQRPG |
| | | QAPRLLIYGASNRATGIPARFSGSGSGTDFTLTISRLEPEDFA |
| | | VYYCGQSYSYPFTFGQGTKLEIK |

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table H.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE H
Amino acid sequence of exemplary anti-GITR
antibody molecules

| | | |
|---|---|---|
| BMS-986156 | | |
| SEQ ID NO: 920 | VH | QVQLVESGGGVVQPGRSLRLSCAASGFTFSSYGMHWVRQAPGK |
| | | GLEWVAVIWYEGSNKYYADSVKGRFTISRDNSKNTLYLQMNSL |
| | | RAEDTAVYYCARGGSMVRGDYYYGMDVWGQGTTVTVSS |
| SEQ ID NO: 921 | VL | AIQLTQSPSSLSASVGDRVTITCRASQGISSALAWYQQKPGKAPK |
| | | LLIYDASSLESGVPSRFSGSGSGTDFTLTISSLQPEDFATYYCQQF |
| | | NSYPYTFGQGTKLEIK |

### IL15/IL-15Ra Complexes

In one aspect of the invention, the IL-1β inhibitor or a functional fragment thereof is administered together with an IL-15/IL-15Ra complex. In some embodiments, the IL-15/IL-15Ra complex is chosen from NIZ985 (Novartis), ATL-803 (Altor) or CYP0150 (Cytune).

In one embodiment, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 1001 in Table I and the soluble form of human IL-15Ra comprises an amino acid sequence of SEQ ID NO: 1002 in Table I, as described in WO 2014/066527, incorporated by reference in its entirety. The molecules described herein can be made by vectors, host cells, and methods described in WO 2007/084342, incorporated by reference in its entirety.

TABLE I
Amino acid and nucleotide sequences of exemplary
IL-15/IL-15Ra complexes

| | | |
|---|---|---|
| NIZ985 | | |
| SEQ ID NO: | Human IL-15 | NWVNVISDLKKIEDLIQSMHIDATLYTESDVHPSCKVTAMKCFLLE |
| 1001 | | LQVISLESGDASIHDTVENLIILANNSLSSNGNVTESGCKECEELEEK |
| | | NIKEFLQSFVHIVQMFINTS |
| SEQ ID NO: | Human Soluble | ITCPPPMSVEHADIWVKSYSLYSRERYICNSGFKRKAGTSSLTECVL |
| 1002 | IL-15Ra | NKATNVAHWTTPSLKCIRDPALVHQRPAPPSTVTTAGVTPQPESLS |
| | | PSGKEPAASSPSSNNTAATTAAIVPGSQLMPSKSPSTGTTEISSHESS |
| | | HGTPSQTTAKNWELTASASHQPPGVYPQG |

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated by reference in its entirety. In one embodiment, the IL-15/IL-15Ra Fc fusion protein comprises the sequences as disclosed in Table J.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated by reference in their entirety. In one embodiment, the IL-15/IL-15Ra sushi domain fusion comprises the sequences as disclosed in Table J.

TABLE J
Amino acid sequences of other exemplary IL-15/
IL-15Ra complexes
ALT-803 (Altor)

| | | |
|---|---|---|
| SEQ ID NO: | IL-15N72D | NWVNVISDLKKIEDLIQSMHIDATLYTESDVHPSCKVTA |
| 1003 | | MKCFLLELQVISLESGDASIHDTVENLIILANDSLSSNGN |
| | | VTESGCKECEELEEKNIKEFLQSFVHIVQMFINTS |
| SEQ ID NO: | IL-15RaSu/Fc | ITCPPPMSVEHADIWVKSYSLYSRERYICNSGFKRKAGTSSLTECVL |
| 1004 | | NKATNVAHWTTPSLKCIREPKSCDKTHTCPPCPAPELLGGPSVFLFP |
| | | PKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSV |
| | | MHEALHNHYTQKSLSLSPGK |

IL-15/IL-15Ra sushi domain fusion (Cytune)

| | | |
|---|---|---|
| SEQ ID | Human IL-15 | NWVNVISDLKKIEDLIQSMHIDATLYTESDVHPSCKVTAMKCFLLE |
| NO: 1005 | | LQVISLESGDASIHDTVENLIILANNSLSSNGNVTESGCKECEELEXK |
| | | NIKEFLQSFVHIVQMFINTS |
| | | Where X is E or K |
| SEQ ID | Human IL- | ITCPPPMSVEHADIWVKSYSLYSRERYICNSGFKRKAGTSSLTECVL |
| NO: 1006 | 15Ra sushi and | NKATNVAHWTTPSLKCIRDPALVHQRPAPP |
| | hinge domains | |

### CTLA-4 Inhibitors

In one aspect of the invention, the IL-1β inhibitor or a functional fragment thereof is administered together with an inhibitor of CTLA-4. In some embodiments, the CTLA-4 inhibitor is an anti-CTLA-4 antibody or fragment thereof. Exemplary anti-CTLA-4 antibodies include Tremelimumab (formerly ticilimumab, CP-675,206); and Ipilimumab (MDX-010, Yervoy®).

In one embodiment, the present invention provides an IL-1β antibody or a functional fragment thereof (e.g., canakinumab or gevokizumab) for use in the treatment of cancers having at least partial inflammatory bases, e.g., lung cancer, especially NSCLC, wherein said IL-1β antibody or a functional fragment thereof is administered in combination with one or more chemotherapeutic agent, wherein said one or more chemotherapeutic agent is a check point inhibitor, preferably selected from the group consisting of nivolumab, pembrolizumab, atezolizumab, avelumab, durvalumab, PDR-001 (spartalizumab) and Ipilimumab. In one embodiment, the one or more chemotherapeutic agent is a PD-1 or PD-L-1 inhibitor, preferably selected from the group consisting of nivolumab, pembrolizumab, atezolizumab, avelumab, durvalumab, PDR-001 (spartalizumab), further preferably pembrolizumab. In one further embodiment, the IL-1β antibody or a functional fragment thereof is administered at the same time of the PD-1 or PD-L1 inhibitor.

In one embodiment, the cancer of the patient has high PD-L1 expression. Typically high PD-L1 expression is defined as Tumor Proportion Score (TPS)≥50%, as determined by an FDA-approved test. In one embodiment, the cancer of the patient has TPS≥1% as determined by an FDA-approved test. In one embodiment, the cancer of the patient has TPS between 1% to 49% as determined by an FDA-approved test. In one embodiment, the cancer of the patient has TPS≥25%, suitably between 25% to 49% as determined by an FDA-approved test.

In one embodiment, the one or more therapeutic agents is alpelisib or a pharmaceutical salt thereof. Alpelisib is administered at a therapeutically effective amount of about 300 mg per day. In one embodiment, DRUG of the invention, suitably canakinumab or gevokizumab, is used in combination with alpelisib in the treatment of cancer, e.g., cancer having at least partial inflammatory basis, selected from the list consisting of TNBC, head and neck cancer, squamous cell carcinoma, and gynecological cancers, including but not limited to cervical, primary peritoneal, ovarian, uterine/endometrial, vaginal and vulvar cancers. In one embodiment, the cancer is breast cancer, suitably hormone receptor (HR)-positive, human epidermal growth factor receptor 2 (HER2)-negative breast cancer, suitably in postmenopausal woman or in man, suitably the cancer is with a PIK3CA mutation, suitably the cancer is advanced breast cancer, suitably after disease progression following an endocrine-based regimen.

In one embodiment, the one or more therapeutic agents is lacnotuzumab. In one embodiment, the one or more therapeutic agents further include a check point inhibitor, suitably a check point inhibitor, suitably selected from pembrolizumab, nivolumab, spartalizumab, atezolizumab, avelumab, ipilimumab, durvalumab. In one embodiment, the cancer is selected from breast cancer, especially TNBC, endometrial, pancreatic carcinoma and melanoma. lacnotuzumab is administered at a dose of 3 mg/kg, 5 mg/kg, 7.5 mg/kg or 10 mg/kg body weight, preferably every 3 weeks or every 4 weeks.

In one embodiment, the one or more chemotherapeutic agents is midostaurin (Rydapt®). In one embodiment, the cancer is acute myeloid leukemia (AML), suitably newly diagnosed AML, suitably the patient bears FLT3 mutation, e.g., as detected by an FDA-approved test. In one embodiment, the one or more chemotherapeutic agents further include cytarabine and daunorubicin, preferably in combination with standard cytarabine and daunorubicin induction and cytarabine consolidation. In one embodiment, midostaurin is administered 50 mg orally twice daily, preferably with food. In a preferred embodiment, midostaurin is administered 50 mg orally twice daily with food on Days 8 to 21 of each cycle of induction with cytarabine and daunorubicin and on Days 8 to 21 of each cycle of consolidation with high-dose cytarabine. In one embodiment, the cancer is AML. In one embodiment, canakinumab is administered about 200 mg every 4 weeks, in combination with midostaurin. In one embodiment, gevokizumab is administered about 30-120 mg every 4 weeks, in combination with midostaurin.

In one embodiment, the one or more chemotherapeutic agents is 5-bromo-2,6-di-(1H-pyrazol-1-yl)pyrimidine-4-amine or a pharmaceutically acceptable salt thereof (the compound described in Example 1 in the PCT publication WO 2011/121418, which is hereby incorporated by reference in its entirety. In one embodiment, the cancer is selected from a list consisting of NSCLC, RCC, prostate cancer, head and neck cancer, TNBC, MSS CRCand melanoma.

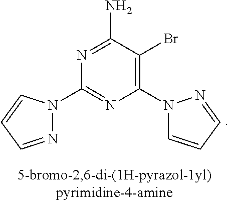 [SMILES: Nc1nc(-n2cccn2)nc(-n2cccn2)c1Br]

In one embodiment, the one or more chemotherapeutic agents is 4-[2-((1R,2R)-2-Hydroxy-cyclohexylamino)-benzothiazol-6-yloxy]-pyridine-2-carboxylic acid methylamide or a pharmaceutically acceptable salt thereof (compound 157 in the PCT publication WO 2007/121484 A2, which is hereby incorporated by reference in its entirety). In one embodiment, the cancer is selected from a list consisting of breast cancer, preferably TNBC, pancreatic cancer, lymphoma and sarcomas of the head and neck.

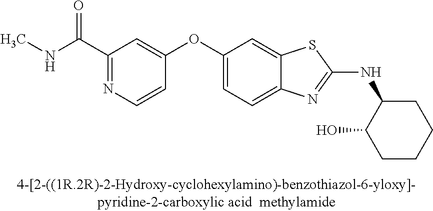 [SMILES: CNC(=O)c1cc(Oc2ccc3nc(N[C@H]4CCCC[C@@H]4O)sc3c2)ccn1]

In one embodiment, the one or more therapeutic agents is HDM2-p53 interaction inhibitor, e.g., (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (WO 2013/111105, example 102) or a pharmaceutically acceptable non-covalent derivative (including salt, solvate, hydrate, complex, co-crystal) thereof, preferably a succinic acid derivative, e.g., succinic acid co-crystal. In one embodiment, the cancer is AML.

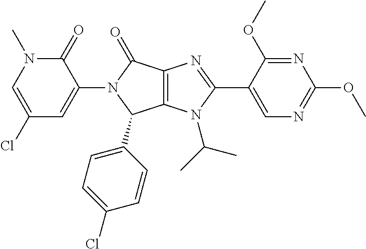 [SMILES: COc1ncc(-c2nc3c(n2C(C)C)[C@H](c2ccc(Cl)cc2)N(c2cc(Cl)cn(C)c2=O)C3=O)c(OC)n1]

In one embodiment, the one or more therapeutic agents is a TGF-beta inhibitor, preferably NIS793.

The heavy chain variable region of NIS793 has the amino acid sequence of:

(SEQ ID NO: 6 in WO 2012/167143)
QVQLVQSGAEVKKPGSSVKVSCKASGGTFSSYAISWVRQAPGQGLEWMGGI
IPIFGTANYAQKFQGRVTITADESTSTAYMELSSLRSEDTAVYYCARGLWE
VRALPSVYWGQGTLVTVSS.

The light chain variable region of NIS793 has the amino acid sequence of:

(SEQ ID NO: 8 in WO 2012/167143)
SYELTQPPSVSVAPGQTARITCGANDIGSKSVHWYQQKAGQAPVLVVSEDI
IRPSGIPERISGSNSGNTATLTISRVEAGDEADYYCQVWDRDSDQYVFGTG
TKVTVLG.

NIS793 is a fully human monoclonal antibody that specifically binds and neutralizes TGF-beta 1 and 2 ligands. In one embodiment, the one or more therapeutic agents further includes one PD-1 or PD-L1 inhibitor, suitably selected from selected from pembrolizumab, nivolumab, spartalizumab, atezolizumab, avelumab, ipilimumab, durvalumab. suitably pembrolizumab, suitably spartalizumab. In one embodiment, the cancer is selected from the list consisting of colorectal cancer (CRC), HCC, NSCLC, breast cancer, prostate cancer, pancreatic cancer and RCC.

In one embodiment, the one or more chemotherapeutic agents is ribociclib or any pharmaceutical salt thereof. In one embodiment, the cancer is breast cancer, suitably hormone receptor (HR)-positive, human epidermal growth factor receptor 2 (HER2)-negative breast cancer, suitably advanced or metastatic breast cancer, suitably in pre/perimenopausal or postmenopausal women, suitably as initial endocrine-based therapy, suitably in combination with aromatase inhibitor.

In one embodiment, the cancer is breast cancer, suitably hormone receptor (HR)-positive, human epidermal growth factor receptor 2 (HER2)-negative breast cancer, suitably advanced or metastatic breast cancer, suitably in postmenopausal women, suitably as initial endocrine-based therapy, suitably in combination with fulvestrant.

In one embodiment, ribociclib is administered at a dose of 600 mg daily for 21 consecutive days followed by 7 days off treatment resulting a 28-day full cycle. In one embodiment, canakinumab is administered 200 mg every 4 weeks, in combination with ribociclib. In one embodiment, gevokizumab is administered 30-120 mg every 4 weeks, in combination with ribociclib.

The term “in combination with” is understood as the two or more drugs are administered subsequently or simultaneously. Alternatively, the term “in combination with” is understood that two or more drugs are administered in the manner that the effective therapeutical concentration of the drugs are expected to be overlapping for a majority of the period of time within the patient's body. The DRUG of the invention and one or more combination partner (e.g., another drug, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The drug administered to a patient as separate entities either simultaneously, concurrently or sequentially with no specific time limits, wherein such administration provides therapeutically effective levels of the two compounds in the body of the patient and the treatment regimen will provide beneficial effects of the drug combination in treating the conditions or disorders described herein. The latter also applies to cocktail therapy, e.g., the administration of three or more active ingredients.

### Administration, Formulations and Devices

Canakinumab can be administered intravenously or preferably subcutaneously. Both administration routes are applicable to each and every canakinumab related embodiments disclosed in this application unless in embodiments wherein the administration route is specified.

Gevokizumab can be administered subcutaneously or preferably intravenously. Both administration routes are applicable to each and every gevokizumab related embodiments disclosed in this application unless in embodiments wherein the administration route is specified.

Canakinumab can be prepared as a medicament in a lyophilized form for reconstitution. In one embodiment, canakinumab is provided in the form of lyophilized form for reconstitution containing at least about 200 mg drug per vial, preferably not more than 250 mg, preferably not more than 225 mg in one vial.

In one aspect the present invention provides canakinumab or gevokizumab for use in treating and/or preventing a cancer in a patient in need thereof, comprising administering a therapeutically effective amount to the patient, wherein the cancer has at least a partial inflammatory basis, and wherein canakinumab or gevokizumab is administered by a prefilled syringe or by an auto-injector. Preferably the prefilled syringe or the auto-injector contains the full amount of therapeutically effective amount of the drug. Preferably the prefilled syringe or the auto-injector contains 200 mg of canakinumab. Preferably the prefilled syringe or the auto-injector contains 250 mg of canakinumab. Preferably the prefilled syringe or the auto-injector contains 50 mg of canakinumab.

### Efficacy and Safety

Due to its good safety profile, canakinumab or gevokizumab can be administered to a patient for a long period of time, providing and maintaining the benefit of suppressing IL-1β mediated inflammation. Furthermore due to its anti-cancer effect, either used in monotherapy or in combination with one or more therapeutic agents, patients life can be extended, including but not limited to extended duration of DFS, PFS, OS, hazard ratio reduction, than without the Treatment of th Invention. The term “Treatment of the Invention”, as used in the this application, refers to DRUG of the invention, suitably canakinumab or gevokizumab, administered according to the dosing regimen, as taught in this application. Preferably the clinical efficacy is achieved at a dose of 200 mg canakinumab administered every 3 weeks or monthly, preferably for at least 6 months, preferably at least 12 months, preferably at least 24 months, preferably up to 2 years, preferably up to 3 years. Preferably the results is achieved at a dose of 30 mg-120 mg gevokizumab administered every 3 weeks or monthly, preferably for at least 6 months, preferably at least 12 months, preferably at least 24 months, preferably up to 2 years, preferably up to 3 years. In one embodiment, Treatment of the Invention is the sole treatment. In one embodiment, Treatment of the Invention is added on top of the SoC treatment for the cancer indication. While the SoC treatment evolves with time, the SoC treatment as used here should be understood as not including DRUG of the invention.

Thus in one aspect the present invention provides an IL-1β binding antibody or functional fragment thereof, suitably canakinumab or gevokizumab, for use in the treatment and/or prevention of cancer, e.g., cancer that has at least a partial inflammatory basis, in a patient, wherein a therapeutically effective amount of an IL-1β binding antibody or a functional fragment thereof is administered in the patient for at least 6 months, preferably at least 12 months, preferably at least 24 months. In one embodiment, the cancer excludes lung cancer, especially excludes NSCLC, especially excludes post-surgery NSCLC, in which the cancer has been resect, suitably not longer than 2 months, preferably not longer than one month.

In one aspect, the present invention provides an IL-1β binding antibody or functional fragment thereof, suitably canakinumab or gevokizumab, for use in the treatment of cancer, e.g., cancer that has at least a partial inflammatory basis, in a patient, wherein the hazard ratio of cancer mortality of the patient is reduced by at least 10%, at least 20%, at least 30%, at least 40% or at least 50%, preferably compared to not receiving Treatment of the Invention.

The term “not receiving Treatment of the Invention”, as used throughout the application, include patients who did not receive any drug at all and patient received only treatment, considered as SoC at the time, without the DRUG of the invention. As a skilled person would understand, the clinical efficacy is typically not tested within the same patient, receiving or not receiving the Treatment of the Invention, rather tested in clinical trial settings with treatment group and placebo group.

In one embodiment, the overall survival (OS, defined as the time from the date of randomization to the date of death due to any cause) in the patient is at least one month, at least 3 months, at least 6 months, at least 12 months longer than not receiving Treatment of the Invention. In one embodiment, the OS is at least 12 months, preferably at least 24 months, longer in the adjuvant treatment setting. In one embodiment, the OS is at least 4 months, preferably at least 6 months, at least 12 months longer in the first line treatment setting. In one embodiment, the OS is at least one month, at least 3 months, preferably at least 6 months longer in the 2^{nd}/3^{rd} line treatment setting.

In one embodiment, the overall survival in the patient receiving Treatment of the Invention is at least 2 years, at least 3 years, at least 5 years, at least 8 years, at least 10 years in the adjuvant treatment setting. In one embodiment, the overall survival in the patient receiving Treatment of the Invention is at least 6 month, at least one year, at least 3 years in the first line treatment setting. In one embodiment, the overall survival in the patient receiving Treatment of the Invention is at least 3 month, at least 6 months, at least one year in the 2^{nd}/3^{rd} line treatment setting.

In one embodiment, the progression free survival (PFS) period of the patient receiving Treatment of the Invention is extended by at least one months, at least 2 months, at least 3 months, at least 6 months, at least 12 months, preferably compared to not receiving Treatment of the Invention. In one embodiment, PFS is extended by at least 6 months, preferably at least 12 months in the first line treatment settings. In one embodiment, PFS is extended by at least one month, at least 3 months, at least 6 months in the second line treatment settings.

In one embodiment, the patient receiving Treatment of the Invention has at least 3 months, at least 6 months, at least 12 months, or at least 24 months progression free survival.

Normally clinical efficacy, including but not limited to DFS, PFS, HR reduction, OS, can be demonstrated in clinical trials comparing treatment group and placebo group. In placebo group patients receive no drug at all or receive SoC treatment. In the treatment group patients receive DRUG of the invention either as monotherapy or added to the SoC treatment. Alternatively in placebo group patient receives SoC treatment and in the treatment group patients receive DRUG of the invention.

Even though the clinical outcome, such as duration of DFS or the HR reduction of cancer mortality, is described as a number based on statistical analysis of a clinical trial, one of ordinary skill would readily extrapolate these statistics to treatments for an individual patient, as claimed, since it is expected the DRUG of the invention would achieve similar clinical outcome in a portion of the individual patients receiving Treatment of the Invention, for example in 95% of the patients, when clinical trials have demonstrated statistical significance (p≤0.05); or for example in 50% of the patients, when clinical trials have provided mean value, such as mean PFS is 24 months

IL-1β blockade could affect patients immune system in combating infection. Thus in one aspect the present invention provides an IL-1β binding antibody or a functional fragment thereof, suitably canakinumab or gevokizumab, for use in the treatment and/or prevention of cancer, e.g., cancer having at least a partial inflammatory basis, and wherein the patient is not at high risk of developing serious infection due to the Treatment of the Invention. Patient would be at high risk of developing serious infection due to the Treatment of the Invention in the following, but not limited to, the following situations: (a) Patient have an active infection requiring medical intervention. The term “active infection requiring medical intervention” is understood as the patient is currently taking or have been taking or have just finished taken for less than one month or less than two weeks, any anti-viral and/or any anti-bacterial medicines; (b) Patient have latent tuberculosis and/or a history of tuberculosis.

To manage the inhibition of the immune system by IL-1β blockade, it is cautioned that the IL-1β binding antibody or a functional fragment thereof is not administered concomitantly with a TNF inhibitor. Preferably a TNF inhibitor is selected from a group consisting of Enbrel® (etanercept), Humira® (adalimumab), Remicade® (infliximab), Simponi® (golimumab), and Cimzia® (certolizumab pegol). It is also cautioned that the IL-1β binding antibody or a functional fragment thereof is not administered concomitantly with another IL-1β blocker, wherein preferably said IL-1 blocker is selected from a group consisting of Kineret® (anakinra) and Arcalyst® (rilonacept). Furthermore it is only one IL-1β binding antibody or a functional fragment thereof is administered in the treatment/prevention of cancer. For example canakinumab is not administered in combination with gevokizumab.

When canakinumab is administered into patients, it is likely that some patients will develop anti-canakinumab antibody (anti-drug antibody, ADA), which needs to be monitored for safety and efficacy reasons. In one aspect the present invention provides canakinumab for use in the treatment and/or preventing cancer, e.g., cancer having at least a partial inflammatory basis, wherein the chance of the patient develops ADA is less than 1%, less than 0.7%, less than 0.5%, less than 0.4%. In one embodiment, the antibody is detected by the method as described in EXAMPLE 11. In one embodiment, the antibody is detection is performed at 3 month, at 6 month or at 12 month from the first administration of canakinumab.

### Exemplar of Cancers to be Treated According to the Present Invention

### RCC

In one aspect the present invention provides an IL-1β binding antibody or a functional fragment thereof, suitably gevokizumab or suitably canakinumab, alone or in combination, for use in the treatment of cancer having at least partial inflammatory basis, wherein said cancer is renal cell carcinoma (RCC). In one aspect the present invention provides an IL-1β binding antibody or a functional fragment thereof for use, alone or in combination, in the treatment of renal cell carcinoma (RCC). The term “renal cell carcinoma (RCC)” as used herein refers to a cancer of the kidney arising from the epithelium of the renal tubules within the renal cortex and includes primary renal cell carcinoma, locally advanced renal cell carcinoma, unresectable renal cell carcinoma, metastatic renal cell carcinoma, refractory renal cell carcinoma, and/or cancer drug resistant renal cell carcinoma. In one embodiment, RCC is renal clear cell carcinoma. In one embodiment, RCC is predominantly clear cell RCC. In one embodiment, gevokizumab or a functional fragment thereof, alone or preferably in combination, is used in the treatment of metastatic RCC.

In one embodiment, the present invention provides DRUG of the invention, preferably canakinumab or gevokizumab, for use in the treatment of renal cell carcinoma (RCC), wherein DRUG of the invention is administered in combination with one or more therapeutic agent, e.g., chemotherapeutic agent or a check point inhibitor. In one embodiment, the therapeutic agent is the standard of care agent for renal cell carcinoma (RCC). In one embodiment, the one or more agent is selected from everolimus (Afinitor®), bevacizumab (Avastin®), bevacizumab with interferon, axitinib (Inlyta®), cabozantinib (Cabometyx®), lenvatinib mesylate (Lenvima®), sorafenib tosylate (Nexavar®), nivolumab (Opdivo®), pazopanib hydrochloride (Votrient®), sunitinib malate (Sutent®), temsirolimus (Torisel®). Depending on the patient condition, one, two or three chemotherapeutic agents can be selected from the list above, to be combined with DRUG of the invention.

In one embodiment, the present invention provides DRUG of the invention, preferably canakinumab or gevokizumab, for use in the neo-adjuvant treatment. Often SoC drug in the neo-adjuvant treatment is the same drug as adjuvant treatment. In one embodiment, the present invention provides DRUG of the invention, preferably canakinumab or gevokizumab, for use in the prevention of recurrence or relapse of RCC, which has been surgically removed (adjuvant treatment). In one embodiment, DRUG of the invention is used in the RCC adjuvant treatment in combination with one or more therapeutic agent. In one embodiment, the one or more therapeutic agent is the SoC in the RCC adjuvant treatment. Often SoC drug in the adjuvant treatment is the same drug as SoC in the first line treatment. SoC of high risk of relapse RCC after surgical resection are sunitinib, pembrolizumab (in investigation), nivolumab+ipilimumab (in investigation). In one embodiment, the one or more therapeutic agent is a TKI, preferably sunitinib or Cabozantinib, further preferably sunitinib. In one embodiment, the one or more therapeutic agent is a check point inhibitor, preferably a PD1 or PD-L1 inhibitor, preferably pembrolizumab, preferably in the dosing interval of every 3 weeks.

In one embodiment, DRUG of the invention is used as monotherapy in the prevention of recurrence or relapse of RCC, which has been surgically removed (adjuvant treatment). This is preferred due to the good safety profile of canakinumab or gevokizumab. In one embodiment, DRUG of the invention is used as monotherapy in the RCC adjuvant treatment after patient has received at least 2 cycles, at least 4 cycles or has completed the intended chemotherapy as adjuvant treatment, suitably the intended chemotherapy is sunitinib.

In one embodiment, DRUG of the invention, preferably canakinumab or gevokizumab is used, alone or preferably in combination, in the first line treatment of renal cell carcinoma (RCC). Preferably DRUG of the invention is used in combination of the SoC drugs, which are approved as the first line treatment of RCC. In one embodiment, the treatment continues until disease progress, preferably according to RECIST 1.1.

The preferred options for first line systemic clear cell RCC are sunitinib, pazopanib, bevacizumab with interferon, and temsirolimus for poor risk patients, avelumab with axitinib, pembrolizumab with axitinib, pembrolizumab with lenvatinib, nivolumab with ipilimumab for patients with intermediate and poor risk metastatic RCC (NCCN Guidelines). The results from the CheckMate 214 study demonstrated that nivolumab plus ipilumumab improved ORR and OS versus sunitinib leading to the recent FDA approval of the combination for the first-line treatment of intermediate and poor risk advanced untreated RCC (Motzer et al 2018). Thus, it is expected that nivolumab with ipilumumab will become the preferred first-line treatment regimen for patients with intermediate and poor risk metastatic RCC. For subsequent therapy for patients with predominantly clear cell RCC, clinical guidelines recommend treatment with cabozantinib, nivolumab, lenvatinib with everolimus and axitinib as preferred option (Bamias et al 2017, NCCN Guidelines 2018).

Cabozantinib, a small-molecule inhibitor of tyrosine kinases such as VEGF, MET and AXL, was explored as second line treatment in the phase III METEOR trial, where 658 patients pre-treated with prior tyrosine kinases inhibitors were randomized (1:1) to 60 mg/d oral cabozantinib or 10 mg/d oral everolimus. Based on the studies conducted, cabozantinib or the immune checkpoint inhibitor, nivolumab, are commonly recommended as a preferred subsequent-line treatment options for patients with clear cell metastatic RCC after failure of prior anti-angiogenic therapy (Jain et al 2017). Since, dual blockade of VEGF and IL-1β signaling in the tumor microenvironment has a potential for synergistic anti-tumor effect by decreasing angiogenesis and modulating the immune response, it is reasonable to use cabozantinib, an inhibitor of tyrosine kinases involved in angiogenesis, as a backbone for combination with gevokizumab patients with metastatic RCC in this study.

In one embodiment, DRUG of the invention, preferably canakinumab or gevokizumab is used, alone or preferably in combination with one ore more therapeutic agent, in second or third line treatment of renal cell carcinoma (RCC). Drugs that have been approved for 2 L or 3 L RCC, particularly predominantly clear cell RCC, includes but not limited to cabozantinib, nivolumab, lenvatinib with everolimus, axitinib, pazopanib, sunitinib and everolimus. In one embodiment, the one ore more therapeutic agent is cabozantinib. In one embodiment, the treatment continues until disease progress, preferably according to RECIST 1.1.

All the uses disclosed throughout this application, including but not limited to, doses and dosing regimens, combinations, route of administration and biomarkers can be applied to the treatment of RCC.

In one embodiment, the present invention provides DRUG of the invention for use in combination of cabozantinib in the treatment of RCC, wherein RCC is advanced second or third line metastatic RCC, preferably have clear-cell component. In one preferred embodiment patient have received one or two lines of systemic treatment, preferably at least one line of treatment has to include anti-angiogenic therapy for at least 4 weeks (single agent or in combination), preferably with radiographic progression during this line of treatment. In one Patients have not received prior cabozantinib. In one embodiment, patients have not received ≥3 lines of systemic therapy for treatment of mRCC. In one embodiment, patients have serum hs-CRP level ≥7 mg/L or preferably ≥10 mg/L. In one embodiment, Cabozantinib is administered at 60 mg orally once daily on a 28 days cycle. Canakinumab is administered at 200 mg of a 28-day cycle or gevokizumab is administered at 30 mg to 120 mg of a 28-day cycle. Patients will continue to receive the treatment until disease progression, preferably per RECIST 1.1.

### CRC

In one aspect, the present invention provides an IL-1β binding antibody or a functional fragment thereof, suitably gevokizumab, suitably canakinumab, alone or in combination, for use in the treatment of cancer having at least partial inflammatory basis, wherein said cancer is colorectal cancer (CRC). In one aspect, the present invention provides an IL-1β binding antibody or a functional fragment thereof, suitably gevokizumab or suitably canakinumab, alone or in combination, for use in the treatment of colorectal cancer. The term “Colorectal cancer (CRC)”, also known as large bowel cancer or colon cancer or rectal cancer, as used herein means a neoplasm arising from the colon and/or rectum, particularly from the epithelium of the colon and/or rectum and includes colon adenocarcinoma, rectal adenocarcinoma, metastatic colorectal cancer (mCRC), advanced colorectal cancer, refractory colorectal cancer, refractory metastatic microsatellite stable (MSS) colorectal cancer unresectable colorectal cancer, and/or cancer drug resistant colorectal cancer. Up to 25% of patients are diagnosed with metastatic disease at presentation and 50% of patients may go on to develop metastases at some point in life.

In one embodiment, the present invention provides DRUG of the invention, preferably canakinumab or gevokizumab, for use in the treatment of CRC, wherein DRUG of the invention is administered in combination with one or more therapeutic agent, e.g., chemotherapeutic agent or a check point inhibitor. In one embodiment, the therapeutic agent is the standard of care agent for CRC. Chemotherapeutic agent is selected from irinotecan hydrochloride (Camptosar®), capecitabine (Xeloda®), oxaliplatin (Eloxatin®), 5-FU (fluorouracil), leucovorin calcium (folinic acid), FU-LV/FL (5-FU plus leucovorin), trifluridine/tipiracil hydrochloride (Lonsurf®), nivolumab (Opdivo®), regorafenib (Stivarga®), FOLFOXIRI (leucovorin, 5-fluorouracil [5-FU], oxaliplatin, irinotecan), FOLFOX (leucovorin, 5-FU, oxaliplatin), FOLFIRI (leucovorin, 5-FU, irinotecan), CapeOx (capecitabine plus oxaliplatin), XELIRI (capecitabine (Xeloda®) plus irinotecan hydrochloride), XELOX (capecitabine (Xeloda®) plus oxaliplatin), FOLFOX plus bevacizumab (Avastin®), cetuximab (Erbitux®), panitumumab (Vectibix®), FOLFIRI plus ramucirumab (Cyramza®), FOLFIRI plus cetuximab (Erbitux®), and FOLFIRI plus Ziv-aflibercept (Zaltrap). Depending on the patient condition, one, two or three therapeutic agents can be selected from the list above, to be combined with gevokizumab or canakinumab.

In one embodiment, the one or more chemotherapeutic agent is a general cytotoxic agent, wherein preferably said general cytotoxic agent is selected from the list consisting of FOLFOX, FOLFIRI, capecitabine, 5-fluorouracil, irinotecan and oxaliplatin.

Usually, the initial therapy of CRC involves a cytotoxic backbone of a doublet chemotherapy regimen, combining fluorouracil and oxaliplatin (FOLFOX), fluorouracil and irinotecan (FOLFIRI), or capecitabine and oxaliplatin (XELOX). Bevacizumab is typically recommended upfront combined with chemotherapy. For patients with wild-type RAS tumors anti-EGFR agents (cetuximab and/orpanitumumab) represent alternative options for initial biologic therapy in combination with backbone chemotherapy.

The anti-EGFR therapies, cetuximab and panitumumab, are restricted to patients with Ras wildtype tumors while bevacizumab may be administered regardless of Ras mutation status.

The term “FOLFOX” as used herein refers to a combination therapy (e.g., chemotherapy) comprising at least one oxaliplatin compound chosen from oxaliplatin, pharmaceutically acceptable salts thereof, and solvates of any of the foregoing; at least one 5-fluorouracil (also known as 5-FU) compound chosen from 5-fluorouracil, pharmaceutically acceptable salts thereof, and solvates of any of the foregoing; and at least one folinic acid compound chosen from folinic acid (also known as leucovorin), levofolinate (the levo isoform of folinic acid), pharmaceutically acceptable salts of any of the foregoing, and solvates of any of the foregoing. The term “FOLFOX” as used herein is not intended to be limited to any particular amounts of or dosing regimens for those components.

The term “FOLFIRI” as used herein refers to a combination therapy (e.g., chemotherapy) comprising at least one irinotecan compound chosen from irinotecan, pharmaceutically acceptable salts thereof, and solvates of any of the foregoing; at least one 5-fluorouracil (also known as 5-FU) compound chosen from 5-fluorouracil, pharmaceutically acceptable salts thereof, and solvates of any of the foregoing; and at least one compound chosen from folinic acid (also known as leucovorin), levofolinate (the levo isoform of folinic acid), pharmaceutically acceptable salts of any of the foregoing, and solvates of any of the foregoing. The term “FOLFIRI” as used herein is not intended to be limited to any particular amounts of or dosing regimens for these components. Rather, as used herein, “FOLFIRI” includes all combinations of these components in any amounts and dosing regimens.

In one embodiment, the one or more chemotherapeutic agent is a VEGF inhibitor (e.g., an inhibitor of one or more of VEGFR (e.g., VEGFR-1, VEGFR-2, or VEGFR-3) or VEGF). Exemplary VEGFR pathway inhibitors that can be used in combination with an IL-1β binding antibody or a functional fragment thereof, suitably gevokizumab, for use in the treatment of cancer, espepially especially cancer with partial inflammatory basis, include, e.g., bevacizumab (also known as rhuMAb VEGF or AVASTIN®), ramucirumab (Cyramza®), and ziv-aflibercept (Zaltrap®). In one preferred embodiment the VEGF inhibitor is bevacizumab. In one embodiment, the one or more chemotherapeutic agent is FOLFIRI plus bevacizumab or FOLFOX plus bevacizumab or XELOX plus bevacizumab.

In one embodiment, the one or more therapeutic agent, e.g., agent is a checkpoint inhibitor, preferably a PD-1 or PD-L1 inhibitor, preferably selected from the group consisting of nivolumab, pembrolizumab, atezolizumab, avelumab, durvalumab and spartalizumab (PDR-001). In one preferred embodiment the one or more therapeutic agent is pembrolizumab. In one preferred embodiment the one or more chemotherapeutic agent is nivolumab.

In one preferred embodiment the one or more therapeutic agent is atezolizumab. In one further preferred embodiment the one or more therapeutic agent, e.g., chemotherapeutic agent is atezolizumab and cobimetinib.

In one preferred embodiment the one or more chemotherapeutic agent is ramucirumab. In one preferred embodiment said patient has metastatic CRC.

In one preferred embodiment the one or more chemotherapeutic agent is ziv-aflibercept. In one preferred embodiment said patient has metastatic CRC.

In one preferred embodiment the one or more chemotherapeutic agent is a a tyrosine kinase inhibitor. In one embodiment, said tyrosine kinase inhibitor is an EGF pathway inhibitor, preferably an inhibitor of Epidermal Growth Factor Receptor (EGFR). In one embodiment, said EGFR inhibitor is cetuximab. In one embodiment, said EGFR inhibitor is panitumumab.

In one embodiment, the present invention provides DRUG of the invention, preferably canakinumab or gevokizumab, for use in the neo-adjuvant treatment. Often SoC drug in the neo-adjuvant treatment is the same drug as adjuvant treatment. In one embodiment, the present invention provides DRUG of the invention, preferably canakinumab or gevokizumab, for use in the prevention of recurrence or relapse of CRC, which has been surgically removed (adjuvant treatment). In one embodiment, DRUG of the invention is used in the CRC adjuvant treatment in combination with one or more therapeutic agent. In one embodiment, the one or more therapeutic agent is the SoC in the CRC adjuvant treatment. Often SoC drug in the adjuvant treatment is the same drug as SoC in the first line treatment. In one embodiment, canakinumab or gevokizumab is used in the CRC adjuvant treatment in combination with fluoropyrimidine and oxaliplatin.

In one embodiment, DRUG of the invention is used as monotherapy in the prevention of recurrence or relapse of CRC, which has been surgically removed (adjuvant treatment). This is preferred due to the good safety profile of canakinumab or gevokizumab. In one embodiment, DRUG of the invention is used as monotherapy in the CRC adjuvant treatment after patient has received at least 2 cycles, at least 4 cycles or has completed the intended chemotherapy as adjuvant treatment, suitably the intended chemotherapy is fluoropyrimidine and oxaliplatin.

In one embodiment, DRUG of the invention, suitably canakinumab or gevokizumab, is used, alone or preferably in combination, in the first line treatment of CRC. Preferably DRUG of the invention is used in combination of the SoC drugs, which is approved as the first line treatment of CRC. The current treatment is with cytotoxic backbone of a doublet chemotherapy regimen, combining a fluoropyrimidine (5-fluorouracil or capecitabine), leucovorin (or levoleucovorin) with either oxaliplatin (in FOLFOX or XELOX regimens) or with irinotecan (in FOLFIRI or XELIRI regimens).

Bevacizumab, cetuximab, and panitumumab are the only targeted therapies currently indicated for the first-line treatment of K-RAS wildtype mCRC in combination with backbone chemotherapies.

The current standard of care in first line mCRC patients with K-Ras wildtype tumors is cetuximab or bevacizumab, in combination with either FOLFOX or FOLFIRI.

In one embodiment, DRUG of the invention, preferably canakinumab or gevokizumab is used, alone or preferably in combination with one ore more therapeutic agent, in second or third line of CRC. For the treatment of second line mCRC, it is recommended that the chemotherapy backbone be switched such that if a patient was treated in the first line with a FOLFOX- or XELOX-based regimen, then FOLFIRI should be used in the second line. Alternatively, if FOLFIRI was used in the first line setting, then FOLFOX or XELOX would be the preferred partner in the second line. Multiple second line studies have demonstrated the benefit of adding an anti-angiogenic agent, such as bevacizumab, to chemotherapy. These data further extended the indication for bevacizumab, allowing for use in the treatment of second-line patients who had progressed on a first-line bevacizumab-containing regimen.

Immune checkpoint inhibitors (pembrolizumab, nivolumab, nivolumab with ipilimumab) are indicated for treatment of microsatellite instability-high (MSI-H) or mismatch repair deficit (dMMR) mCRC that has progressed following treatment with fluoropyrimidine (5-FU or capecitabine), oxaliplatin, and irinotecan (i.e. after 2 lines of treatment).

In one embodiment, gevokizumab or canakinumab is used in the first line mCRC treatment, wherein patients have had no prior systemic treatment for metastatic intent and no prior adjuvant therapy (except as radiosensitizer). In one embodiment, patients with first line mCRC have hs-CRP≥10 mg/L. In one embodiment, patients with first line mCRC have hs-CRP<10 mg/L. Subjects enrolled in Parts 1a/1b who were administered gevokizumab at the RDE will be included in the Part 2 subject numbers and analysis. In one embodiment, gevokizumab or canakinumab is used in combination with FOLFOX and bevacizumab. Bevacizumab administered at 5 mg/kg IV on day 1 and 15 of a 28 day cycle. FOLFOX (also known as modified FOLFOX6): oxaliplatin administered at 85 mg/m2 IV, leucovorin (folinic acid) 400 mg/m2 IV, and bolus 5-fluorouracil 400 mg/m2 IV followed by 2400 mg/m2 as a 46-h continuous infusion on day 1 and 15 of a 28 day cycle. The treatment is continued until disease progression, preferably per RECIST 1.1.

In one embodiment, gevokizumab or canakinumab is used in the second line mCRC, wherein patients have progressed on or been intolerant to one prior line of chemotherapy in the metastatic disease setting. In one embodiment, patients with second line mCRC have hs-CRP≥10 mg/L. In one embodiment, patients with second line mCRC have hs-CRP<10 mg/L. In one embodiment, the prior line chemotherapy includes at least a fluoropyrimidine and oxaliplatin. Rechallenge with oxaliplatin is permitted and considered part of the first-line regimen for metastatic disease. Both the initial oxaliplatin treatment and the subsequent rechallenge are considered as one regimen. In one embodiment, patients have had no prior exposure to irinotecan. In one embodiment, patients have no history of Gilbert's Syndrome, or any of the following genotypes: UGT1A1*6/*6, UGT1A1*28/*28, or UGT1A1*6/*28. In one embodiment, gevokizumab or canakinumab is used in combination with FOLFIRI and bevacizumab. Bevacizumab administered at 5 mg/kg IV on day 1 and 15 of a 28 day cycle. FOLFIRI: irinotecan administered at 180 mg/m2 IV, leucovorin (folinic acid) 400 mg/m2 IV, and bolus 5-fluorouracil 400 mg/m2 IV followed by 2400 mg/m2 as a 46-h continuous infusion on day 1 and 15 of a 28 day cycle. Canakinumab is administered at 200 mg of a 28-day cycle or gevokizumab is administered at 30 mg to 120 mg of a 28-day cycle.

All the uses disclosed throughout this application, including but not limited to, doses and dosing regimens, combinations, route of administration and biomarkers can be applied to the treatment of CRC.

### Gastric

In one aspect the present invention provides an IL-1β antibody or a functional fragment thereof, suitably gevokizumab or canakinumab, alone or in combination, for use, alone or in combination, in the treatment of gastric cancer.

As used herein, the term “gastric cancer” encompasses gastric cancer and cancer of the esophagus (gastroesophageal cancer), particularly the lower part of the esophagus and refers to primary gastric cancer, metastatic gastric cancer, metastatic esophageal cancer, refractory gastric cancer, unresectable gastric cancer, unresectable esophageal cancer, and/or cancer drug resistant gastric cancer. The term “gastric cancer” includes adenocarcinoma of the distal esophagus, gastroesophageal junction and/or stomach. In a preferred embodiment, the gastric cancer or esophageal cancer are gastroesophageal cancer. In one embodiment, gevokizumab or canakinumab is used in the treatment of metastatic gastric cancer.

In one embodiment, the present invention provides DRUG of the invention, suitably gevokizumab or canakinumab, for use in the treatment of gastric cancer, wherein DRUG of the invention is administered in combination with one or more therapeutic agent, e.g., chemotherapeutic agent. In one embodiment, the therapeutic agent, e.g., chemotherapeutic agent is the standard of care agent for gastric cancer. In one embodiment, the one or more therapeutic agent is selected from the group consisting of carboplatin plus paclitaxel (Taxol®), cisplatin plus 5-fluorouracil (5-FU), ECF (epirubicin (Ellence®), cisplatin, and 5-FU), DCF (docetaxel (Taxotere®), cisplatin, and 5-FU), cisplatin plus capecitabine (Xeloda®), oxaliplatin plus 5-FU, oxaliplatin plus capecitabine, irinotecan (Camptosar®) ramucirumab (Cyramza®), docetaxel (Taxotere®), trastuzumab (Herceptin®), FU-LV/FL (5-fluorouracil plus leucovorin), and XELIRI (capecitabine (Xeloda®) plus irinotecan hydrochloride). Depending on the patient condition, one, two or three therapeutic agents can be selected from the list above, to be combined with gevokizumab or canakinumab.

Patients with unresectable or metastatic gastric and/or gastroesophageal junction adenocarcinoma are candidates for palliative chemotherapy-based treatment only. First-line treatments include platinum agents (cisplatin, oxaliplatin, or carboplatin) and fluoropyrimidines (5-fluorouracil [5-FU], capecitabine), sometimes with the addition of a third drug such as an anthracycline (doxorubicin or epirubicin) or a taxane (paclitaxel or docetaxel) (Pericay 2016). In one embodiment, the one or more therapeutic agents is platinum agents and fluoropyrimidines, with or without anthracycline, with or without taxane.

In one embodiment, the one or more therapeutic agents is Ramucirumab (fully human mAb against the VEGF receptor (VEGFR)-2).

In one embodiment, the one or more therapeutic agents is trastuzumab.

In one embodiment, the one or ore chemotherapeutic agent is paclitaxel. In one embodiment, the one or ore chemotherapeutic agent is ramucirumab. In one embodiment, the one or ore chemotherapeutic agent is paclitaxe and ramucirumab. In one further embodiment said combination is used for second line treatment of metastatic gastroesophageal cancer.

In one embodiment, the one or more therapeutic agent is a checkpoint inhibitor, wherein preferably is a PD-1 or PD-L1 inhibitor, wherein preferably selected from the group consisting of nivolumab, pembrolizumab, atezolizumab, avelumab, durvalumab and spartalizumab (PDR-001). In one embodiment, the one or more therapeutic agent is pembrolizumab.

In one embodiment, the one or more therapeutic agent is nivolumab. In one embodiment, the one or more chemotherapeutic agent is nivolumab plus and ipilimumab. In one further embodiment said combination is used for first or second line treatment of metastatic gastroesophageal cancer.

In one embodiment, the present invention provides DRUG of the invention, preferably canakinumab or gevokizumab, for use in the prevention of recurrence or relapse of gastric cancer, which has been surgically removed (adjuvant treatment). In one embodiment, DRUG of the invention is used in the gastric adjuvant treatment in combination with one or more therapeutic agent. In one embodiment, the one or more therapeutic agent is the SoC in the gastric adjuvant treatment. Often SoC drug in the adjuvant treatment is the same drug as SoC in the first line treatment. In one embodiment, the one or more therapeutic agent in gastric adjuvant treatment is platinum agents (cisplatin, oxaliplatin, or carboplatin) and fluoropyrimidines (5-fluorouracil [5-FU], capecitabine).

In one embodiment, the present invention provides DRUG of the invention, preferably canakinumab or gevokizumab, for use in the neo-adjuvant treatment. Often SoC drug in the neo-adjuvant treatment is the same drug as adjuvant treatment. In one embodiment, DRUG of the invention is used as monotherapy in the prevention of recurrence or relapse of gastric cancer, which has been surgically removed (adjuvant treatment). This is preferred due to the good safety profile of canakinumab or gevokizumab. In one embodiment, DRUG of the invention is used as monotherapy in the gastric adjuvant treatment after patient has received at least 2 cycles, at least 4 cycles or has completed the intended chemotherapy as adjuvant treatment, suitably the intended chemotherapy is platinum agents and fluoropyrimidines.

In one embodiment, DRUG of the invention, suitably canakinumab or gevokizumab, is used, alone or preferably in combination, in the first line treatment of gastric cancer, preferably in combination with one or more therapeutic agents, preferably SoC drugs, which is approved as the first line treatment in gastric cancer. In one embodiment, one or more therapeutic agents is trastuzumab. Trastuzumab is indicated as first line treatment (in combination with chemotherapy without anthracyclines) for Her-2-positive metastatic gastric cancer. In one embodiment, one or more therapeutic agents is platinum agents and fluoropyrimidines.

In one embodiment, DRUG of the invention, preferably canakinumab or gevokizumab is used, alone or preferably in combination with one ore more therapeutic agents, in second or third line of gastric cancer. In one embodiment, the one ore more therapeutic agents is ramucirumab. Ramucirumab, either as a single agent or in combination with paclitaxel, has now been adopted as a standard treatment option in second line metastatic gastroesophageal junction and gastric adenocarcinomas. In one embodiment, the one ore more therapeutic agents is pebrolizumab. Pembrolizumab for PD-L1 [Combined Positive Score (CPS)≥1] metastatic gastroesophageal cancer with disease progression on or after two or more prior lines of therapy including fluoropyrimidine- and platinum-containing chemotherapy, and if appropriate, HER2-targeted therapy.

All the disclosed uses throughout this application, including but not limited to, doses and dosing regimens, combinations, route of administration and biomarkers can be applied to the treatment of gastric cancer.

In one embodiment, gevokizumab or canakinumab is used for the second line treatment of metastatic gastroesophageal cancer, wherein patients have locally advanced, unresectable or metastatic gastric or gastroesophageal junction adenocarcinoma, typically not squamous cell or typically undifferentiated gastric cancer, which has progressed on or been intolerant to first-line systemic therapy. In one embodiment, the first-line systemic therapy is any platinum/fluoropyrimidine doublet, with or without anthracycline (epirubicin or doxorubicin). In one embodiment, the patient has not received other chemotherapy. In one embodiment, the patient has not received any previous systemic therapy targeting VEGF or the VEGFR signaling pathways. Other prior targeted therapies are permitted, if stopped at least 28 days prior to randomization. In one embodiment patient has Serum hs-CRP level ≥10 mg/L. In one embodiment, gevokizumab or canakinumab is combined with paclitaxel and ramucirumab. Ramucirumab is administered at 8 mg/kg IV on day 1 and 15 of a 28 day cycle. Paclitaxel is administered at 80 mg/m2 IV on days 1, 8, and 15 of a 28-day cycle. Canakinumab is administered at 200 mg of a 28-day cycle or gevokizumab is administered at 30 mg to 120 mg of a 28-day cycle. Patients will continue to receive the treatment until disease progression, preferably per RECIST 1.1.

### Melanoma

In one aspect the present invention provides an IL-1β antibody or a functional fragment thereof, suitably gevokizumab or a functional fragment thereof, suitably canakinumab or a functional fragment thereof, for use in the treatment of melanoma. The term “melanoma” includes “malignant melanoma” and “cutaneous melanoma” and as used herein refers to a malignant tumor arising from melanocyte which are derived from the neural crest. Although most melanomas arise in the skin, they may also arise from mucosal surfaces or at other sites to which neural crest cells migrate. As used herein, the term “melanoma” includes primary melanoma, locally advanced melanoma, unresectable melanoma, BRAF V600 mutated melanoma, NRAS-mutant melanoma, metastatic melanoma (including unresectable or metastatic BRAF V600 mutated melanoma), refractory melanoma (including relapsed or refractory BRAF V600-mutant melanoma (e.g., said melanoma being relapsed after failure of BRAFi/MEKi combination therapy or refractory to BRAFi/MEKi combination therapy), cancer drug resistant melanoma (including BRAF-mutant melanoma resistant to BRAFi/MEKi combination treatment) and/or immuno-oncolocy (10) refractory melanoma. In one embodiment, gevokizumab or canakinumab, alone or preferably in combination, is used in the treatment of metastatic melanoma.

Tumor cells expressing the IL-1β precursor must first activate caspase-1 in order to process the inactive precursor into active cytokine. Activation of caspase-1 requires autocatalysis of procaspase-1 by the nucleotide-binding domain and leucine-rich repeat containing protein 3 (NLRP3) inflammasome (Dinarello, C. A. (2009). Ann Rev Immunol, 27, 519-550). In late-stage human melanoma cells, spontaneous secretion active IL-1β is observed via constitutive activation of the NLRP3 inflammasome (Okamoto, M. et al The Journal of Biological Chemistry, 285, 6477-6488). Unlike human blood monocytes, these melanoma cells require no exogenous stimulation. In contrast, NLRP3 functionality in intermediate stage melanoma cells requires activation of the IL-1 receptor by IL-1α in order to secrete active IL-1β. The spontaneous secretion of IL-1β from melanoma cells was reduced by inhibition of caspase-1 or the use of small interfering RNA directed against the inflammasome component ASC. Supernatants from melanoma cell cultures enhanced macrophage chemotaxis and promoted in vitro angiogenesis, both prevented by pretreating melanoma cells with inhibitors of caspases-1 or IL-1 receptor blockade (Okamoto, M. et al The Journal of Biological Chemistry, 285, 6477-6488). Furthermore, in a screen of human melanoma tumor samples, copy number greater than 1,000 for IL-1β was present in 14 of 16 biopsies, whereas none expressed IL-1α (Elaraj, D. M. et al, Clinical Cancer Research, 12, 1088-1096. Taken together these findings implicate IL-1-mediated autoinflammation, especially IL-1β, as contributing to the development and progression of human melanoma.

In one embodiment, the present invention provides DRUG of the invention, suitably canakinumab or gevokizumab, for use in the treatment of melanoma, in combination with one or more therapeutic agents, e.g., a chemotherapeutic agent, e.g., a check point inhibitor. In one embodiment, the therapeutic agent is the standard of care agent for melanoma. In one embodiment, the one or more therapeutics agent is selected from aldesleukin (Proleukin®), Talimogene Laherparepvec (Imlygic®), (peg)interferon alfa-2b (Intron A®/Sylatron™) Trametinib (Mekinist®), Dabrafenib (Tafinlar®), Trametinib (Mekinist®) plus Dabrafenib (Tafinlar®), cobimetinib (Cotellic®), vemurafenib (Zelboraf®), cobimetinib+vemurafenib, binimetinib (Mektovi®)+encorafenib (Braftovi®), pembrolizumab (Keytruda®), Nivolumab (Opdivo®), Ipilimumab (Yervoy®), Nivolumab (Opdivo®) plus Ipilimumab (Yervoy®). Other medicaments currently being development for the treatment of melanoma include spartalizumab (PDR001), spartalizumab (PDR001)+dabrafenib+trametinib, pembrolizumab+dabrafenib+trametinib, atezolizumab (Tecentriq®) and atezolizumab (Tecentriq®) plus bevacizumab (Avastin®). Depending on the patient condition, one, two or three therapeutic agents can be selected from the list above, to be combined with gevokizumab or canakinumab. Immunotherapies offer significant benefit to melanoma cancer patients, including those for whom conventional treatments are ineffective. Pembrolizumab and nivolumab, two inhibitors of the PD-1/PD-L1 interaction have been approved for use in melanoma. However, results indicate that many patients treated with single agent PD-1 inhibitors do not benefit adequately from treatment. Combination with additional one or more chemotherapeutic agent would normally improve the treatment efficacy.

In one embodiment, the one or more therapeutic agents is nivolumab.

In one embodiment, the one or more ctherapeutic agents is ipilimumab.

In one embodiment, the one or more therapeutic agents is nivolumab and ipilimumab.

In one embodiment, the one or more chemotherapeutic agents is trametinib.

In one embodiment, the one or more chemotherapeutic agents is Dabrafenib.

In one embodiment, the one or more chemotherapeutic agents is trametinib and dabrafenib. In one further embodiment the one or more chemotherapeutic agents is trametinib and dabrafenib, plus pembrolizumab or spartalizumab.

In one embodiment, the one or more chemotherapeutic agents is Pembrolizumab.

In one embodiment, the one or more chemotherapeutic agents is Atezolizumab.

In one embodiment, the one or more chemotherapeutic agents is atezolizumab (Tecentriq®) plus bevacizumab.

In one embodiment, the present invention provides DRUG of the invention, preferably canakinumab or gevokizumab, for use in the neo-adjuvant treatment. Often SoC drug in the neo-adjuvant treatment is the same drug as adjuvant treatment. In one embodiment, the present invention provides DRUG of the invention, preferably canakinumab or gevokizumab, for use in the prevention of recurrence or relapse of melanoma, which has been surgically removed (adjuvant treatment). In one embodiment, DRUG of the invention is used in the melanoma adjuvant treatment in combination with one or more therapeutic agents. In one embodiment, the one or more therapeutic agents is the SoC drug in the melanoma adjuvant treatment. Often SoC drug in the adjuvant treatment is the same drug as SoC in the first line treatment.

In one embodiment, canakinumab or gevokizumab is used as monotherapy in the prevention of recurrence or relapse of melanoma, which has been surgically removed (adjuvant treatment). This is preferred due to the good safety profile of canakinumab or gevokizumab. In one embodiment, canakinumab or gevokizumab is used as monotherapy in the melanoma adjuvant treatment after patient has received at least 2 cycles, at least 4 cycles or has completed the intended chemotherapy as adjuvant treatment.

In one embodiment, DRUG of the invention, preferably canakinumab or gevokizumab is used, alone or preferably in combination, in the first line treatment of melanoma. Preferably canakinumab or gevokizumab is used in combination of the SoC drugs, which are approved as the first line treatment of melanoma.

In one embodiment, DRUG of the invention, preferably canakinumab or gevokizumab is used, alone or preferably in combination with one ore more therapeutic agent, in second or third line treatment of melanoma.

All the disclosed uses throughout this application, including but not limited to, doses and dosing regimens, combinations, route of administration and biomarkers can be applied to the treatment of melanoma.

### Bladder

In one aspect the present invention provides an IL-1β binding antibody or a functional fragment thereof, suitably gevokizumab or canakinumab, for use in the treatment of bladder cancer. The term “bladder cancer” as used herein refers to transitional cell carcinoma of the bladder, urothelial (cell) carcinoma, i.e. carcinomas of the urinary bladder, ureter, renal pelvis and urethra. The term includes reference to the non-muscle-invasive (NMI) or superficial forms, as well as to the muscle invasive (MI) types. The term includes three main types of bladder cancer: Urothelial carcinoma, squamous cell carcinoma, or adenocarcinoma. Also included in the term is reference to primary bladder cancer locally advanced bladder cancer unresectable bladder cancer, metastatic bladder cancer, refractory bladder cancer, relapsed bladder cancer and/or cancer drug resistant bladder cancer.

Recent studies have linked inflammation with the formation and development of bladder cancer (Sui et al., Oncotarget. 2017).

In one embodiment, gevokizumab or canakinumab, alone or preferably in combination with one or more therapeutic agents, is used in the treatment of metastatic bladder cancer.

All the disclosed uses throughout this application, including but not limited to, doses and dosing regimens, combinations, route of administration and biomarkers can be applied to the treatment of bladder cancer.

Treatment regimens of bladder cancer include intravesical therapy for early stages of bladder cancer as well as chemotherapy with and without radiation therapy.

In one embodiment, the present invention provides DRUG of the invention, suitably gevokizumab or canakinumab, for use in the treatment of bladder cancer, in combination with one or more therapeutic agents, e.g a chemotherapeutic agent or e.g., a check point inhibitor. In one embodiment, the therapeutic agent is the standard of care agent for bladder cancer. In one embodiment, the one or more therapeutic agent is selected from cisplatin, cisplatin plus fluorouracil (5-FU), mitomycin plus 5-FU, gemcitabine plus cisplatin, MVAC (methotrexate, vinblastine, doxorubicin (adriamycin), plus cisplatin), CMV (cisplatin, methotrexate, and vinblastine), carboplatin plus paclitaxel or docetaxel, gemcitabine, cisplatin, carboplatin, docetaxel, paclitaxel, doxorubicin, 5-FU, methotrexate, vinblastine, ifosfamide, pemetrexed, thiotepa, valrubicin, atezolizumab (Tecentriq®), avelumab (Bavencio®), durvalumab (Imfinzi®), pembrolizumab (Keytruda®) and nivolumab (Opdivo®). Depending on the patient condition, one, two or three chemotherapeutic agents can be selected from the list above, to be combined with gevokizumab or canakinumab.

In one embodiment, the one or more therapeutic agents is a checkpoint inhibitor, wherein preferably is a PD-1 or PD-L1 inhibitor, wherein preferably selected from the group consisting of nivolumab, pembrolizumab, atezolizumab, avelumab, durvalumab and spartalizumab (PDR-001), preferably nivolumab or preferably pembrolizumab.

In one embodiment, the present invention provides DRUG of the invention, preferably canakinumab or gevokizumab, for use in the neo-adjuvant treatment. In one embodiment, the present invention provides DRUG of the invention, preferably canakinumab or gevokizumab, for use in the prevention of recurrence or relapse of bladder cancer, which has been surgically removed (adjuvant treatment). In one embodiment, DRUG of the invention is used in the bladder adjuvant treatment in combination with one or more therapeutic agents. In one embodiment, the one or more therapeutic agent is the SoC in the bladder adjuvant treatment. Often SoC drug in the neo-adjuvant treatment is the same drug as adjuvant treatment. Often SoC drug in the adjuvant treatment is the same drug as SoC in the first line treatment. In one embodiment, the one or more therapeutic agents is methotrexate, vinblastine, doxorubicin and cisplatin (known as DDMVAC (dose-dense methotrexate, vinblastine, doxorubicin and cisplatin) with growth factor support, suitably for 3-4 treatment cycles. In one embodiment, the one or more therapeutic agents is Gemcitabine and cisplatin, suitably for 4 cycles. In one embodiment, DRUG of the invention is used as monotherapy in the prevention of recurrence or relapse of bladder cancer, which has been surgically removed (adjuvant treatment). This is preferred due to the good safety profile of canakinumab or gevokizumab. In one embodiment, DRUG of the invention is used as monotherapy in the bladder adjuvant treatment after patient has received at least 2 cycles, at least 4 cycles or has completed the intended chemotherapy as adjuvant treatment.

In one embodiment, DRUG of the invention, preferably canakinumab or gevokizumab is used, alone or preferably in combination, in the first line treatment of bladder cancer. Preferably DRUG of the invention is used in combination of the SoC drugs, which are approved as the first line treatment of bladder cancer. In one embodiment, the treatment continues until disease progress, preferably according to RECIST 1.1. In one embodiment, the one or more therapeutic agents is gemcitabine and cisplatin or DDMVAC with growth factor support, suitably for cisplatin eligible patient. In one embodiment, the one or more therapeutic agents is gemcitabine and carboplatin, gemcitabine, gemcitabine+paclitaxel, atezolizumab or pembrolizumab, suitably for cisplatin ineligible patient.

In one embodiment, DRUG of the invention, preferably canakinumab or gevokizumab, is used, alone or preferably in combination with one ore more therapeutic agents, in the second or third line treatment of bladder cancer. In one embodiment, the treatment continues until disease progress, preferably according to RECIST 1.1. In one embodiment, the one or more therapeutic agents are a check point inhibitors, suitably selected from pembrolizumab, atezolizumab, nivolumab, durvalumab and avelumab, suitably as the 2^{nd} line of treatment. Post check point inhibitor 2^{nd}/3^{rd} line treatment include gemcitabine/carboplatin for cisplatin ineligible, chemotherapy naïve patients, gemcitabine+cisplatin OR DDMVAC with growth factor support for cisplatin eligible, chemotherapy naïve patient, Nab-paclitaxel, Paclitaxel or docetaxel and Pemetrexed.

### Prostate

In one aspect, the present invention provides an IL-1β binding antibody or a functional fragment thereof, suitably gevokizumab or suitably canakinumab, alone or in combination with one or more therapeutic agents, for use in the treatment of cancer, e.g., cancer having at least partial inflammatory basis, wherein the cancer is prostate cancer.

Pre-clinically IL-1β mediated pathways (particularly via IL-8 expression) have been implicated in prostate cancer cell proliferation and migration (Tsai et al., J Cell Biochem. 2009: 108(2): 489-98). In addition, IL-1β has been shown to induce prostate cancer neuroendocrine differentiation (NED) in vitro and promote both skeletal colonization and growth of metastatic cell lines in mice (Chiao et al., Int J Oncol. 1999; 15(5): 1033-7). Furthermore, IL-1β has been directly implicated in the development of androgen independent prostate cancer cells that have reduced or no dependence on androgen for survival (Chang et al., J Cell Biochem. 2014; 115(12):2188-2197).

While emerging research has identified a number of molecular sub-types of prostate cancer, clinical guidelines continue to divide therapy largely by risk stratification based on a combination of the TNM classification, PSA levels, biopsy and Gleason score.

The most significant sub-typing of prostate cancer is based on disease progression, in particular prostate tumors sensitivity to androgen deprivation therapy which is the 1^{st} line standard of care:
Castration naïve represents those patients who are not on ADT at the time of progression
Castrate resistant represents is cancer that progresses clinically despite testosterone being <50 ng/dL

Prostate cancer can be further categorized by their cellular origin. For example, Adenocarcinomas (e.g., Acinar adenocarcinoma) are cancers that develop in the gland cells that line the prostate gland. They are the most common type of prostate cancer. Ductal adenocarcinoma starts in the cells that line the ducts (tubes) of the prostate gland. It tends to grow and spread more quickly than acinar adenocarcinoma. Transitional cell (or urothelial) cancer of the prostate starts in the cells that line the tube carrying urine to the outside of the body (the urethra). This type of cancer usually starts in the bladder and spreads into the prostate, but rarely it can start in the prostate and may spread into the bladder entrance and nearby tissues. Squamous cell cancers develop from flat cells that cover the prostate. They tend to grow and spread more quickly than adenocarcinoma of the prostate. Small cell prostate cancer is made up of small round cells. It is a type of neuroendocrine cancer. Prostate cancer can also be metastatic. The term “prostate cancer” as used herein covers all types and stages thereof.

In one embodiment, the present invention provides DRUG of the invention, preferably canakinumab or gevokizumab, alone or preferably in combination with one or more therapeutic agents, for use in the treatment of metastatic prostate cancer.

In one embodiment, the present invention provides DRUG of the invention, preferably canakinumab or gevokizumab, for use in the treatment of prostate cancer, wherein DRUG of the invention is administered in combination with one or more therapeutic agents, e.g., a chemotherapeutic agent, a targeted therapy agent, a cell-based therapy or a checkpoint inhibitor or a combination of these agents.

The above therapy can be further administered in combination with radiation therapy, suitably EBRT (External Beam Radiotherapy). The above therapy can be further administered in combination with androgen deprivation therapy (ADT), with or without radiotherapy.

In one embodiment, the one or more therapeutic agents is a chemotherapeutic agent, e.g., selected from Cabazitaxel, Mitoxantrone Hydrochloride, Radium 223 Dichloride, platinums, fluorouracil (5-FU), erbitux, taxanes, bleomycin, ifosfamide, vinblastine, gemcitabine, navelbine, iressa, tarceva, BIBW, paclitaxel, docetaxel, and methotrexate.

In one embodiment, the one or more therapeutic agents is a targeted therapy agent selected from EGFR inhibitors, e.g., antibodies, e.g., panitumumab and cetuximab, or tyrosine kinase inhibitors, e.g., afatinib, erlotinib, gefitinib, and lapatinib; VEGF inhibitors e.g., antibodies, e.g., bevacizumab, ranibizumab, or VEGFR inhibitors, e.g., lapatinib, sunitinib, sorafenib, axitinib and pazopanib; mTOR Inhibitors, e.g., everolimus; or MET or HGF inhibitors.

In one embodiment, the one or more therapeutic agents is an androgen deprivation therapy (ADT), such as LHRH agonists or antagonists, e.g., leuprorelin, goserelin, triptorelin, histrelin, buserelin, and degarelix; or antiandrogens, e.g., cyproterone acetate, flutamide, nilutamide, bicalutamide, enzalutamide, abiraterone acetate, seviteronel, apalutamide, and darolutamide.

In one embodiment, the one or more therapeutic agent is a checkpoint inhibitor, selected from a list consisting of pembrolizumab, nivolumab, spartalizumab, atezolizumab, avelumab, ipilimumab and durvalumab.

In one embodiment, the one or more therapeutic agent is a cell-based cancer immunotherapy, e.g., Sipuleucel-T.

Depending on the patient condition, one, two, three or four of the therapeutic agents can be selected from the above lists to be combined with DRUG of the invention.

In one embodiment, the present invention provides DRUG of the invention, preferably canakinumab or gevokizumab, for use in the treatment of prostate cancer, wherein DRUG of the invention is administered in combination with a combination of one or more chemotherapeutic agents and one or more targeted therapy agents, a combination of one or more chemotherapeutic agents and one or more checkpoint inhibitors, a combination of one or more chemotherapeutic agents and one or more targeted therapy agents and one or more checkpoint inhibitors.

In one embodiment, the one or more therapy or therapeutic agent is ADT, preferably apalutamide or enzalutamide. In a preferred embodiment, the prostate cancer is a Castration resistant prostate cancer (M0—no distant metastasis).

In one embodiment, the one or more therapy or therapeutic agent is ADT, preferably apalutamide or enzalutamide. In a preferred embodiment, this is combined with Denosumab or zoledronic acid, and/or immunotherapy with sipuleucel-T, and/or palliative radiotherapy. In a preferred embodiment, the prostate cancer is a Castration resistant prostate cancer (M1—metastasis to distant organs).

In one embodiment, the one or more therapy or therapeutic agent is ADT, preferably apalutamide or enzalutamide. In a preferred embodiment, this is combined with one or more of the drugs selected from the group consisting of abiraterone and prednisone, docetaxel, enzalutamide, Radium-223 (for bone metastases), abiraterone and methylprednisolone, or any other secondary hormone therapy. In a preferred embodiment, the prostate cancer is a Castration resistant prostate cancer (M1—metastasis to distant organs), more preferably wherein no visceral metastasis is present or detected or diagnosed to be present.

In one embodiment, the one or more therapeutic agent is docetaxel, Radium-223 (for bone metastases), preferably wherein the prostate cancer is a Castration resistant prostate cancer (M1) post progression, more preferably without visceral metastases, more preferably wherein the prior therapy has been abiraterone and/or enzalutamide.

In one embodiment, the one or more therapy or therapeutic agent is abiraterone with prednisone, cabazitaxel, enzalutamide, Radium-223, abiraterone with methylprednisolone, Sipuleucel-T (if not received), docetaxel re-challenge, mitoxantrone with prednisone, or other secondary hormone therapy. In a preferred embodiment, the prostate cancer is a Castration resistant prostate cancer (M1—metastasis to distant organs), more preferably wherein no visceral metastasis is present or detected or diagnosed to be present, more preferably wherein the prior therapy has been docetaxel.

In one embodiment, the one or more therapy or therapeutic agent is chemotherapy (e.g., cisplatin/etoposide or carboplatin/etoposide or docetaxel/carboplatin), docetaxel, abiraterone & prednisone or abiraterone & methylprednisolone or enzalutamide or cabazitaxel (if not previously received) or secondary hormone therapy. In a preferred embodiment, the prostate cancer is a small cell cancer. In a preferred embodiment, the prostate cancer is a castration resistant prostate cancer (M1) post progression, more preferably wherein visceral metastases is present or detected or diagnosed to be present.

In one embodiment, the one or more therapy or therapeutic agent is a 1^{st} line therapy, preferably docetaxel or enzalutamide or abiraterone & prednisone or abiraterone & methylprednisolone or clinical trial or mitoxantrone & prednisone or other secondary hormone therapy. In a preferred embodiment, the prostate cancer is an Adenocarcinoma. In a preferred embodiment, the prostate cancer is a castration resistant prostate cancer (M1) post progression, more preferably wherein visceral metastases is present or detected or diagnosed to be present.

In one embodiment, the one or more therapy or therapeutic agent is a 2^{nd} line therapy, preferably abiraterone & prednisone or enzalutamide or cabazitaxel or abitraterone & methylprednisolone or docetaxel rechallenge or mitoxantrone with prednisone. In a preferred embodiment, the prostate cancer is an Adenocarcinoma. In a preferred embodiment, the prostate cancer is a castration resistant prostate cancer (M1) post progression, more preferably wherein visceral metastases is present or detected or diagnosed to be present.

In one preferred embodiment the one or more therapy or therapeutic agent is orchiectomy or LHRH agonist (e.g., Goserelin, histrelin, leuprolide, triptorelin), optionally in combination with antiandrogen or LHRH antagonist. In a preferred embodiment, the prostate cancer is M0—no distant metastases, more preferably Castration-naïve.

In one preferred embodiment the one or more therapy or therapeutic agent is ADT in combination with docetaxel, or ADT in combination with abiraterone and prednisone, or orchiectomy, or LHRH optionally in combination with antiandrogen or LHRH antagonist or ADT in combination with abiraterone with methylprednisolone. In a preferred embodiment, the prostate cancer is M1—distant metastases, more preferably Castration-naïve.

In one preferred embodiment the one or more therapy or therapeutic agent is EBRT optionally in combination with ADT. Preferably, the prostate cancer has not metastasized to distant organs. More preferably, the prostate cancer is in PSA persistence/recurrence stage, more preferably progressing after radical prostatectomy (RP).

In one preferred embodiment the one or more therapy or therapeutic agent is ADT optionally in combination with EBRT. Preferably, the prostate cancer is metastasized in weight bearing joints or symptomatic. More preferably, the prostate cancer is in PSA persistence/recurrence stage, more preferably progressing after radical prostatectomy (RP).

In one preferred embodiment the one or more therapy or therapeutic agent is radical prostatectomy (RP) in combination with pelvic lymph node dissection (PLND) or cryosurgery or Ultrasound or brachytherapy. In a preferred embodiment, the prostate cancer is TRUS (Transrectal ultrasound) positive, wherein no metastases is present or detected or diagnosed to be present. More preferably, the prostate cancer is in PSA persistence/recurrence stage, more preferably progressing after radiation therapy.

In one preferred embodiment the one or more therapy or therapeutic agent is ADT. In a preferred embodiment, the prostate cancer is TRUS (Transrectal ultrasound) negative, wherein no metastases is present or detected or diagnosed to be present. More preferably, the prostate cancer is in PSA persistence/recurrence stage, more preferably progressing after radiation therapy.

In one embodiment, the one or more therapeutic agents is the standard of care (SoC) agent for prostate cancer.

In one embodiment, DRUG of the invention is used in the prostate cancer treatment in combination with one or more therapeutic agents, further in combination with EBRT and/or ADT.

In one embodiment, the present invention provides DRUG of the invention, preferably canakinumab or gevokizumab, for use in the neo-adjuvant treatment. Often SoC drug in the neo-adjuvant treatment is the same drug as adjuvant treatment. In one embodiment, the present invention provides DRUG of the invention, preferably canakinumab or gevokizumab, for use in the prevention of recurrence or relapse of prostate cancer, which has been surgically removed (adjuvant treatment). In one embodiment, DRUG of the invention is used as monotherapy in the adjuvant treatment. This is preferred due to the good safety profile of canakinumab or gevokizumab. In one embodiment, DRUG of the invention is used, in combination with one or more therapies or therapeutic agents, in the adjuvant treatment. In a preferred embodiment, the additional therapy is EBRT, preferably wherein no lymph node metastasis is present or detected or diagnosed to be present. In a preferred embodiment, the additional therapy or therapeutic agent is ADT optionally in combination with EBRT, preferably wherein lymph node metastasis is present or detected or diagnosed to be present.

In one embodiment, one or more therapeutic agent is the SoC in the prostate cancer adjuvant treatment. Often the SoC drug in the adjuvant treatment is the same drug as SoC in the first line treatment.

In one embodiment, DRUG of the invention is used as monotherapy in the prostate cancer adjuvant treatment after the patient has received ADT and/or EBRT or has completed the intended chemotherapy as adjuvant treatment.

In one embodiment, DRUG of the invention is used in the prostate cancer adjuvant treatment in combination at the same time as EBRT and/or ADT.

In one embodiment, DRUG of the invention, preferably canakinumab or gevokizumab is used, alone or preferably in combination with one or more therapeutic agents, in the first line treatment of prostate cancer. In one embodiment, the one or more therapeutic agents is a therapeutic agent used as first line treatment selected from Abiraterone Acetate, Apalutamide, Bicalutamide, Cabazitaxel, Degarelix, Docetaxel, Leuprolide Acetate, Enzalutamide, Flutamide, Goserelin Acetate, Mitoxantrone Hydrochloride, Nilutamide, Radium 223 Dichloride, Sipuleucel-T.

In one embodiment, DRUG of the invention, preferably canakinumab or gevokizumab is used, alone or preferably in combination with one or more therapies or therapeutic agent, in second or third line treatment of prostate cancer. In one embodiment, the one or more therapies or therapeutic agents is selected from orchiectomy or LHRH agonist (e.g., Goserelin, histrelin, leuprolide, triptorelin), optionally in combination with antiandrogen or LHRH antagonist. In one embodiment, the one or more therapies or therapeutic agents is selected from ADT in combination with docetaxel, or ADT in combination with abiraterone and prednisone, or orchiectomy, or LHRH optionally in combination with antiandrogen or LHRH antagonist or ADT in combination with abiraterone with methylprednisolone. In one embodiment, the one or more therapies or therapeutic agents is EBRT optionally in combination with ADT. In one embodiment, the one or more therapies or therapeutic agents is selected from radical prostatectomy (RP) in combination with pelvic lymph node dissection (PLND) or cryosurgery or Ultrasound or brachytherapy.

In one embodiment, the treatment, e.g., the adjuvant treatment, the first line treatment or the 2^{nd} or 3^{rd} line treatment continues until disease progress, preferably according to RECIST 1.1.

All the uses disclosed throughout this application, including but not limited to, doses and dosing regimens, combinations, route of administration and biomarkers can be applied to the treatment of prostate cancer.

### Breast

It as an object of the present invention to provide further treatment options for use in breast cancer. Current breast cancer treatment includes the treatment of local disease with surgery, radiation therapy or both, and systemic treatment with chemotherapy, endocrine therapy, check point inhibitor therapy (or immunotherapy) or a combination thereof. All the uses disclosed throughout this application, including but not limited to, doses and dosing regimens, combinations, route of administration and biomarkers can be applied to the treatment of breast cancer.

The term “breast cancer” as used herein includes cancer of the breast, regardless of origin, for example arising in ducts (ductal carcinoma, including invasive ductal carcinoma and ductal carcinoma in situ (DCIS) and glands (lobular carcinoma, including invasive lobular carcinoma and lobular carcinoma in situ), and Paget's disease of the breast, and includes, but is not limited to, estrogen-receptor-positive (ER+) breast cancer, estrogen-receptor-negative (ER−) breast cancer, progesterone-receptor-positive (PR+) breast cancer, progesterone-receptor-negative (PR−) breast cancer, HER2-receptor positive (HER2+) breast cancer, HER2-receptor negative (HER2−) breast cancer, ER+/PR+,HER2+ breast cancer, ER−/PR+,HER2+ breast cancer, ER+/PR−,HER2+ breast cancer, ER+/PR+,HER2− breast cancer, ER−/PR+,HER2− breast cancer, ER+/PR−,HER2− breast cancer, ER−/PR−,HER2+ breast cancer, and triple negative breast cancer (TNBC; a breast cancer that is HER2−, ER− and PR−) according to established clinical guidelines. The breast cancer may also be inflammatory breast cancer or metastatic breast cancer.
In models of breast cancer a number of publications have implicated IL-1β in both early and late stages of metastasis. (Mans et al., PLoS Med. 2015; Oh et al., BMC Cancer. 2016; Guo et al., Sci Rep. 2016).
IL-1β has been implicated in tumor immuno-suppression, supporting a role for a IL-1β binding antibodies or fragments thereof, such as canakinumab or gevokizumab, in improving efficacy of existing check point inhibitors, especially in HR−/HER2− (TNBC) tumors (adjuvant, 1st line and refractory metastatic breast cancer) and HR+/HER2− tumors (1st line metastatic breast cancer)
In a cell model of breast cancer cells it was shown that IL-1β induces Epithelial-Mesenchymal Transition (EMI) by activation of the IL-1β/IL-1RI/β-catenin pathway, resulting in methylation of the ESR1 gene promoter. This epigenetic modification produced significant decrease of the ERα receptor levels and increased resistance to tamoxifen. Unspecifically blocking the PI3K/AKT signalling pathway with wortmannin restored sensitivity of the cells to tamoxifen (Jimenez-Garduno et al., Biochem Biophy Res Commun. 2017; 490(3):780-785). Therefore, IL-1β inhibition can be utilized in combination with ERα targeted therapies (tamoxifen, fulvestrant, SERDs) in the adjuvant and 1st line metastatic breast cancer setting in estrogen receptor positive tumors.
IL-1β was also shown to up-regulate BIRC3 which has been implicated in doxorubicin resistance (Mendoza-Rodriguez et al., Cancer Lett. 2017; 390:39-44). Inhibition of IL-1β by canakinumab or gevokizumab can therefore be used in combination with dose-dense doxorubicin/cyclophosphamide (AC) to prevent resistance in an adjuvant setting or in TNBC where doxorubicin is a preferred agent.
IL-1β is known to be elevated after chemotherapy with various agents such as cisplatin, vincrisitine, etoposide, paclitaxel, methotrexate, 5-FU, and gemcitabine and may drive progression of disease (Bent et al., Int J Mol Sci. 2018; 19: 2155-2189). IL-1β inhibition can therefore be utilized as a post-chemotherapy maintenance therapy across adjuvant, first line and recurrent metastatic breast cancer in HR−/HER2− patients and in the adjuvant setting for HR+/HER2− patients.
It is expected that the addition of the IL-1β binding antibody or a functional fragment thereof, suitably gevokizumab or suitably canakinumab, provides therapeutic benefits exceeding the current standard of care by blocking IL-1β signalling implicated in angiogenesis, lymphangiogenesis, primary breast tumor growth, invasion, metastasis and/or immunosuppression pathways within the breast cancer tumor micro-environment.
Accordingly, In one embodiment, the present invention provides an IL-1β binding antibody or a functional fragment thereof, suitably gevokizumab or suitably canakinumab, alone or in combination with one or more therapeutic agents, for use in the inhibition or prevention of metastasis in breast cancer.
In one embodiment, the present invention provides DRUG of the invention, preferably canakinumab or gevokizumab, for use in the neo-adjuvant treatment. Often SoC drug in the neo-adjuvant treatment is the same drug as adjuvant treatment. In another embodiment the present invention provides an IL-1β binding antibody or a functional fragment thereof, suitably gevokizumab or suitably canakinumab, for use in the prevention of recurrence or relapse of breast cancer, which has been surgically removed (adjuvant treatment). In one embodiment, the IL-1β binding antibody or a functional fragment thereof, suitably gevokizumab or suitably canakinumab is used in breast cancer adjuvant treatment in combination with one or more therapeutic agent. In one embodiment, the one or more therapeutic agent is the SoC in breast cancer adjuvant treatment. In one embodiment, the IL-1β binding antibody or a functional fragment thereof, suitably gevokizumab or suitably canakinumab is used as monotherapy in the prevention of recurrence or relapse of breast cancer, which has been surgically removed (adjuvant treatment). This is preferred due to the good safety profile of canakinumab or gevokizumab. In one embodiment, the IL-1β binding antibody or a functional fragment thereof, suitably gevokizumab or suitably canakinumab, is used as monotherapy in breast cancer adjuvant treatment after said patient has received at least 2 cycles, at least 4 cycles or has completed the intended therapy as adjuvant treatment, suitably the intended therapy is chemotherapy or endocrine therapy or radiotherapy or a combination of any of these. In another aspect, the present invention provides an IL-1β binding antibody or a functional fragment thereof, suitably gevokizumab or suitably canakinumab, either as monotherapy or in combination with at least one further therapeutic agent, for use in post-radiation adjuvant therapy.
In one embodiment, the one or more therapeutic agent for any breast cancer related embodiment described herein, unless specifically stated otherwise, is selected from methotrexate, abraxane (paclitaxel albumin-stabilized nanoparticle formulation), aminoglutethimide, anastrozole, pamidronate disodiumrozole, bevacizumab, capecitabine, carboplatin, cisplatin, cyclophosphamide, docetaxel, pegylated liposomal doxorubicin, docetaxel trihydrate, epirubicin hydrochloride, eribulin mesylate, etirinotecan pegol, exemestane, fadrozole, fluorouracil (5-FU), formestane, fulvestrant, gemcitabine hydrochloride, goserelin acetate, ibandronic acid, ixabepilone, lapatinib ditosylate (Tyverb®/Tykerb®), letrozole, megestrol acetate, methotrexate, neratinib maleate (Nerlynx®), olaparib, paclitaxel, pamidronate disodium, poziotinib, tamoxifen, talazoparib, testolactone, thiotepa, toremifene, vinblastine sulfate, vinorelbine, vorozole, AC (doxorubicin hydrochloride (adriamycin) and cyclophosphamide), AC-T (doxorubicin hydrochloride (adriamycin), cyclophosphamide and paclitaxel), CAF (cyclophosphamide, doxorubicin hydrochloride (adriamycin) and fluorouracil), CMF (cyclophosphamide, methotrexate and fluorouracil), FEC (fluorouracil, epirubicin hydrochloride, cyclophosphamide), TAC (docetaxel (Taxotere), doxorubicin hydrochloride (adriamycin), cyclophosphamide), palbociclib, abemaciclib, ribociclib, everolimus, trastuzumab (Herceptin®), ado-trastuzumab emtansine (Kadcyla®), pertuzumab (Perjeta®), or a checkpoint inhibitor such as nivolumab, pembrolizumab, atezolizumab, avelumab, durvalumab, spartalizumab (PDR-001), and ipilimumab. In one embodiment, the one or more therapeutic agent is a checkpoint inhibitor, wherein preferably is a PD-1 or PD-L1 inhibitor, wherein said checkpoint inhibitor is selected from the group consisting of nivolumab, pembrolizumab, atezolizumab, avelumab, durvalumab and spartalizumab, preferably pembrolizumab or preferably nivolumab. Depending on the patient condition, one, two or more therapeutic agents can be selected from the list above, to be combined with gevokizumab or canakinumab.
In one aspect the present invention provides an IL-1β binding antibody or a functional fragment thereof, suitably gevokizumab or suitably canakinumab, alone or in combination, for use as a neoadjuvant in breast cancer treatment. In one embodiment, IL-1β binding antibody or a functional fragment thereof, suitably gevokizumab or suitably canakinumab, is used in breast cancer neoadjuvant treatment in combination with one or more therapeutic agent. In one embodiment, the one or more therapeutic agent is the standard of care (SoC) agent in breast cancer neoadjuvant treatment. In another aspect the present invention provides an IL-1β binding antibody or a functional fragment thereof, suitably gevokizumab or suitably canakinumab, alone or in combination, for use as monotherapy or in combination with at least one further therapeutic agent as 1^{st} line treatment in metastatic breast cancer (mBC). In yet another aspect the present invention provides an IL-1β binding antibody or a functional fragment thereof, suitably gevokizumab or suitably canakinumab, alone or in combination, for use as monotherapy or in combination with at least one further therapeutic agent in recurrent metastatic breast cancer. In one embodiment, the present invention provides an IL-1β binding antibody or a functional fragment thereof, suitably gevokizumab or suitably canakinumab, for use in the treatment of breast cancer, wherein the IL-1β binding antibody or a functional fragment thereof is administered in combination with one or more therapeutic agent, e.g., chemotherapeutic agent or a check point inhibitor. In one embodiment, the therapeutic agent, e.g., chemotherapeutic agent, is the standard of care agent for breast cancer.
Standard of care agent in breast cancer is dependent on a variety of factors, including, but not limited to age of the patient, menopausal status, clinical and pathologic characteristics of the primary tumor, hormone receptor content, intrinsic subtype of the cancer, TNM stage, and tumor histology, such as defined in the clinical practice guidelines by the European Society for Medical Oncology (ESMO) (e.g., Senkus et al, Annals of Oncology 26 (Supplement 5): v8-v30, 2015), American Joint Committee on Cancer (AJCC) (e.g., Hortobagyi et al, AJCC Cancer Staging Manual, Eighth Edition, Breast. 10.1007/978-3-319-40618-3_48), the World Health Organisation (WHO) (e.g., Lakhani et al, WHO Classification of Tumours of the Breast, 4th Edition, Volume 4, 2012), and National Comprehensive Cancer Network (NCCN) (e.g., NCCN Clinical Practice Guidelines in Oncology, Breast Cancer, 2018), all of which are hereby incorporated by reference in their entirety.
In one embodiment, IL-1β binding antibody or a functional fragment thereof, suitably gevokizumab or suitably canakinumab, is used, alone or preferably in combination, in first line treatment of breast cancer. Preferably, the IL-1β binding antibody or a functional fragment thereof, suitably gevokizumab or suitably canakinumab, is used in combination with SoC drug(s), which are approved as the first line treatment of breast cancer.
In another aspect, the present invention provides an IL-1β binding antibody or a functional fragment thereof, suitably gevokizumab or suitably canakinumab, in combination with at least one further therapeutic agent, wherein at least one further therapeutic agent is selected from nivolumab, pembrolizumab, atezolizumab, avelumab, durvalumab, spartalizumab, and ipilimumab, and wherein the efficacy of the combination is greater than the efficacy of the at least one further therapeutic agent alone. Optionally, and in addition at least one further therapeutic agent is selected from methotrexate, abraxane (paclitaxel albumin-stabilized nanoparticle formulation), aminoglutethimide, anastrozole, pamidronate disodiumrozole, bevacizumab, capecitabine, carboplatin, cisplatin, cyclophosphamide, docetaxel, pegylated liposomal doxorubicin, docetaxel trihydrate, epirubicin hydrochloride, eribulin mesylate, etirinotecan pegol, exemestane, fadrozole, fluorouracil (5-FU), formestane, fulvestrant, gemcitabine hydrochloride, goserelin acetate, ibandronic acid, ixabepilone, lapatinib ditosylate (Tyverb®/Tykerb®), letrozole, megestrol acetate, methotrexate, neratinib maleate (Nerlynx®), olaparib, paclitaxel, pamidronate disodium, poziotinib, tamoxifen, talazoparib, testolactone, thiotepa, toremifene, vinblastine sulfate, vinorelbine, vorozole, AC (doxorubicin hydrochloride (adriamycin) and cyclophosphamide), AC-T (doxorubicin hydrochloride (adriamycin), cyclophosphamide and paclitaxel), CAF (cyclophosphamide, doxorubicin hydrochloride (adriamycin) and fluorouracil), CMF (cyclophosphamide, methotrexate and fluorouracil), FEC (fluorouracil, epirubicin hydrochloride, cyclophosphamide), TAC (docetaxel (Taxotere), doxorubicin hydrochloride (adriamycin), cyclophosphamide, palbociclib, abemaciclib, ribociclib, everolimus, trastuzumab (Herceptin®), ado-trastuzumab emtansine (Kadcyla®) and pertuzumab (Perjeta®). In a preferred embodiment, such a combination is used in the treatment of TNBC breast cancer. The combination may be used as adjuvant treatment, as first line treatment or in the treatment of refractory metastatic breast cancer. In another embodiment, such a combination is used in the treatment of HR+/HER2− breast cancer, optionally as first line treatment of metastatic HR+/HER2− breast cancer.
In another aspect, the present invention provides an IL-1β binding antibody or a functional fragment thereof, suitably gevokizumab or suitably canakinumab, in combination with at least two further therapeutic agents, wherein at least one further therapeutic agent is selected from nivolumab, pembrolizumab, atezolizumab, avelumab, durvalumab, spartalizumab, and ipilimumab, and wherein at least one further therapeutic agent is selected from methotrexate, abraxane (paclitaxel albumin-stabilized nanoparticle formulation), aminoglutethimide, anastrozole, pamidronate disodiumrozole, bevacizumab, capecitabine, carboplatin, cisplatin, cyclophosphamide, docetaxel, pegylated liposomal doxorubicin, docetaxel trihydrate, epirubicin hydrochloride, eribulin mesylate, etirinotecan pegol, exemestane, fadrozole, fluorouracil (5-FU), formestane, fulvestrant, gemcitabine hydrochloride, goserelin acetate, ibandronic acid, ixabepilone, lapatinib ditosylate (Tyverb®/Tykerb®), letrozole, megestrol acetate, methotrexate, neratinib maleate (Nerlynx®), olaparib, paclitaxel, pamidronate disodium, poziotinib, tamoxifen, talazoparib, testolactone, thiotepa, toremifene, vinblastine sulfate, vinorelbine, vorozole, AC (doxorubicin hydrochloride (adriamycin) and cyclophosphamide), AC-T (doxorubicin hydrochloride (adriamycin), cyclophosphamide and paclitaxel), CAF (cyclophosphamide, doxorubicin hydrochloride (adriamycin) and fluorouracil), CMF (cyclophosphamide, methotrexate and fluorouracil), FEC (fluorouracil, epirubicin hydrochloride, cyclophosphamide), TAC (docetaxel (Taxotere), doxorubicin hydrochloride (adriamycin), cyclophosphamide, palbociclib, abemaciclib, ribociclib, everolimus, trastuzumab (Herceptin®), ado-trastuzumab emtansine (Kadcyla®) and pertuzumab (Perjeta®).
Current standard of care therapeutic agents in adjuvant therapy are outlined in the 2018 National Comprehensive Cancer Network (NCCN) guidelines for breast cancer (version 3.2018). These include the agents as summarized in 3.

TABLE 3
Therapeutic regimen and standard of care drugs according to NCCN
guidelines for breast cancer 2018 as adjuvant therapy in breast cancer

| | Therapeutic regimen | Current SoC drugs |
|---|---|---|
| | according to NCCN | according to NCCN |
| Tumor sub- | guidelines for breast | guidelines for breast |
| type | cancer 2018 | cancer 2018 |
| HR+/HER2− | Endocrine therapy OR | Endocrine: AI (Anastrozole, |
| (Luminal A) | adjuvant chemotherapy | letrozole, exemestane) OR |
| | followed by endocrine | SERM (Tamoxifen) |
| | therapy (radiation | Preferred chemotherapy |
| | therapy also deemed | regimen: Dose |
| | acceptable) | dense AC |
| | | (doxorubicin/cyclophosphamide) |
| | | followed by paclitaxel |
| | | OR docetaxel |
| | | and cyclophosphamide |
| HR+/HER2+ | Depending on tumor | HER2 targeted therapy: |
| (Luminal B) | size and extent of | Trastuzumab |
| | metastasis, the HER2 | (Herceptin) and |
| | targeted therapy | pertuzumab (Perjeta) |
| | trastuzumab is | TKI: Neratinib (Nerlynx) |
| | recommended as a | Preferred chemotherapy |
| | mainstay therapy in | regimen: AC |
| | combination with | followed by paclitaxel + |
| | endocrine therapy, | trastuzumab |
| | chemotherapy and the | +/− pertuzumab |
| | addition of another | OR paclitaxel + |
| | HER2 targeted therapy, | trastuzumab OR |
| | pertuzumab (while not | TCH (docetaxel/ |
| | in the guidelines, | carboplatin/trastuzumab) +/− |
| | neratinib, a TKI was | pertuzumab |
| | recently approved for | |
| | adjuvant setting | |
| | following trastuzumab) | |
| HR−/HER2+ | Adjuvant | HER2 targeted |
| | chemotherapy in | therapy: Trastuzumab |
| | combination with | (Herceptin) and |
| | HER2 targeted | pertuzumab (Perjeta) |
| | therapies trastuzumab | Preferred chemotherapy |
| | with the addition of | regimen: AC |
| | pertuzumab in node | followed by paclitaxel + |
| | positive tumors | trastuzumab |
| | (ipsilateral metastases | +/− pertuzumab OR |
| | >2 mm) | paclitaxel + trastuzumab |
| | | OR TCH (docetaxel/ |
| | | carboplatin/trastuzumab) +/− |
| | | pertuzumab |
| HR−/HER2 | Adjuvant | Preferred chemotherapy |
| (Basal like/ | chemotherapy only | regimen: Dose |
| TNBC) | | dense AC (doxorubicin/ |
| | | cyclophosphamide) followed by |
| | | paclitaxel OR docetaxel and |
| | | cyclophosphamide |

In yet another aspect the present invention provides an IL-1β binding antibody or a functional fragment thereof, suitably gevokizumab or suitably canakinumab, in combination with at least one further therapeutic agent in accordance with the therapeutic regimen selected from Table for use in the adjuvant treatment of breast cancer.
First-line treatment for luminal A type breast cancer is endocrine therapy. Endocrine therapy are anti-hormonal agents, which work in two ways: (1) by lowering the amount of the hormone in the body or (2) by blocking the action of hormone on cells. Various types of anti-hormonal agents are known. One type of anti-hormonal agents is known as aromatase inhibitors. Aromatase inhibitors work by inhibiting the action of the enzyme aromatase, which converts androgens into estrogens by a process called aromatization. As breast tissue is stimulated by estrogens, decreasing their production is a way of suppressing recurrence of the breast tumor tissue. The main source of estrogen is the ovaries in premenopausal women, while in post-menopausal women most of the body's estrogen is produced in peripheral tissues (outside the CNS), and also a few CNS sites in various regions within the brain. Estrogen is produced and acts locally in these tissues, but any circulating estrogen, which exerts systemic estrogenic effects in men and women, is the result of estrogen escaping local metabolism and spreading to the circulatory system. There are two types of aromatase inhibitors: (1) steroidal inhibitors, such as exemestane (Aromasin) which forms a permanent and deactivating bond with the aromatase enzyme; and (2) non-steroidal inhibitors, such as anastrozole (Arimidex) or Letrozole (Femara) which inhibit the synthesis of estrogen via reversible competition for the aromatase enzyme. Another type of anti-hormonal agent is estrogen receptor antagonist. An example of an estrogen receptor antagonist is fulvestrant (Faslodex). Estrogen receptors are found in and on breast cells. Estrogen binds to estrogen receptors, like a key fitting into a lock. This can activate the receptor and cause hormone receptor-positive tumors to grow. Fulvestrant binds to and blocks estrogen receptors and reduces the number of estrogen receptors in breast cells. Another type of anti-hormonal agent is selective estrogen receptor modulators (SERMs) are a class of compounds that act on the estrogen receptor. A characteristic that distinguishes these substances from pure receptor agonists and antagonists is that their action is different in various tissues, thereby granting the possibility to selectively inhibit or stimulate estrogen-like action in various tissues An example of a SERM is tamoxifen.
In another aspect, the present invention provides an IL-1β binding antibody or a functional fragment thereof, suitably gevokizumab or suitably canakinumab, in combination with at least one further therapeutic agent targeting the ERα receptor, for example selected from selective estrogen receptor degrader (SERD), such as fulvestrant, NVS-LSZ102, AZD9496, GDC-0927, elacestrant, SAR-439859, brilanestrant and/or selective estrogen receptor modulators (SERM), such as tamoxifen, toremifen. Optionally, such a combination may be combined with at least one further therapeutic agent, for example a non-steroidal aromatase inhibitor such as anastrazole, letrozole and/or a steroidal aromatase inhibitor such as exemestane and/or everolimus. In a preferred embodiment, such a combination may be used in the treatment of ER positive breast cancer, in particular as an adjuvant and/or in a first line metastatic breast cancer setting.
Accordingly, In one embodiment, the present invention provides IL-1β binding antibody or a functional fragment thereof, suitably gevokizumab or suitably canakinumab for use in combination with endocrine therapy in the treatment of breast cancer, wherein breast cancer is hormone receptor (HR)-positive/HER2-negative breast cancer, comprising administering 200 mg of canakinumab or 30 mg to 120 mg gevokizumab every three weeks or every four weeks (monthly) in combination with endocrine therapy selected from non-steroidal aromatase inhibitor (anastrazole, letrozole), SERD (fulvestrant, NVS-LSZ102, AZD9496, GDC-0927, elacestrant, SAR-439859, brilanestrant), SERM (tamoxifen, toremifen), steroidal aromatase inhibitor (exemestane).
In another aspect, the present invention provides an IL-1β binding antibody or a functional fragment thereof, suitably gevokizumab or suitably canakinumab, for prevention of resistance to anthracycline used as monotherapy or in combination with at least one further therapeutic agent. Anthracycline includes, but is not limited to, doxorubicin, epirubicin, daunorubicin and mitoxantrone, which is used as monotherapy or in combination chemotherapy, for example with cyclophosphamide, in particular in the treatment of TNBC.
In another aspect, the present invention provides an IL-1β binding antibody or a functional fragment thereof, suitably gevokizumab or suitably canakinumab, for use in post chemotherapy maintenance therapy. In a particular embodiment, such maintenance therapy is used in the adjuvant, first-line or recurrent metastatic TNBC. In another embodiment, such maintenance therapy is used as an adjuvant in HR+/HER2− breast cancer.
Current standard of care therapeutic agents in first line treatment of metastatic breast cancer therapy are outlined in the 2018 National Comprehensive Cancer Network (NCCN) guidelines for breast cancer (version 3.2018). These include the agents as summarized in 4.

TABLE 4
Therapeutic regimen and standard of care drugs according
to NCCN guidelines for breast cancer 2018
as first line treatment of metastatic breast cancer

| | Therapeutic regimen | Current SoC drugs |
|---|---|---|
| | according to NCCN | according to NCCN |
| Tumor sub- | guidelines for breast | guidelines for breast |
| type | cancer 2018 | cancer 2018 |
| HR+/HER2− | For pre-menopausal women | Endocrine: Non steroidal |
| (Luminal A) | endocrine therapy +/− a | AI (anastrozole, letrozole), |
| | CDK4/6 inhibitor is | steroidal AI (exemestane) |
| | recommended (together with | and SERM (Tamoxifen) |
| | agent for ovarian suppression, | CDK 4/6 inhibitor: |
| | e.g., goserelin) | abemaciclib, palbociclib, |
| | For post menopausal women | ribociclib |
| | with no prior endocrine | SERD: fulvestrant |
| | therapy, combination of | |
| | CDK4/6 inhibitor with either | |
| | an AI or fulvestrant | |
| | recommended (single agent | |
| | AI or ER modulator/ down- | |
| | regulator also included in the | |
| | guideline) | |
| | For post-menopausal women | |
| | with prior endocrine therapy | |
| | switching to a different AI +/− | |
| | addition of a CDK 4/6 | |
| | inhibitor is recommended | |
| HR+/HER2+ | For pre/post menopausal | Endocrine: Non steroidal |
| (Luminal B) | women consider a different | AI (anastrozole, letrozole), |
| | line of endocrine therapy +/− | steroidal AI (exemestane) |
| | HER2-targeted chemotherapy | HER2 targeted therapies: |
| | OR chemotherapy and HER2− | Trastuzumab (Herceptin), |
| | targeted therapy with the | Pertuzumab (Perjeta) |
| | preferred approach of | Taxane: Docetaxel, |
| | combining pertuzumab with | paclitaxel |
| | trastuzumab and a taxane | |
| HR−/HER2+ | Chemotherapy and HER2 | HER2 targeted therapies: |
| | targeted therapy with the | Trastuzumab (Herceptin) |
| | preferred approach of | and Pertuzumab (Perjeta) |
| | combining pertuzumab with | Taxane: Docetaxel, |
| | trastuzumab and a taxane | paclitaxel |
| HR−/HER2 | Chemotherapy until | Chemotherapy regimen: |
| (Basal like/ | progression is currently stated | No specific |
| TNBC) | within the NCCN guidelines | recommendations or |
| | PARP inhibitors approved for | combinations |
| | gBRCA mutation patients in | PARP: olaparib, |
| | mBC | talazopanib |

In yet another aspect the present invention provides an IL-1β binding antibody or a functional fragment thereof, suitably gevokizumab or suitably canakinumab, in combination with at least one further therapeutic agent in accordance with the therapeutic regimen selected from Table for use in first line treatment of metastatic breast cancer.
Tumor development is closely associated with genetic alteration and deregulation of cyclin dependent kinases (CDKs) and their regulators. Several clinical trials have demonstrated the efficacy of the addition of a CDK4/6 inhibitor to endocrine therapy in hormone receptor (HR)-positive/HER2-negative advanced breast cancer.

Findings from MONALEESA-7, the first dedicated trial investigating a CDK4/6 inhibitor in pre- and peri-menopausal women with hormone receptor (HR)-positive, HER2-negative advanced breast cancer, demonstrated that addition of ribociclib (Kisqali®) to first-line endocrine therapy with tamoxifen/non-steroidal aromatase inhibitor (NSAI) plus goserelin, significantly prolonged progression-free survival (PFS), leading to approval of ribociclib in combination with an aromatase inhibitor for pre/perimenopausal women with HR-positive, HER2-negative advanced or metastatic breast cancer, as initial endocrine-based therapy.

The findings from MONALEESA-3 have led to the approval of the combination of ribociclib and fulvestrant in 1st/2nd line in men and post-menopausal women with HR-positive/HER2-negative advanced breast cancer, providing a significant increase in progression-free survival (PFS).
Similarly, findings from the PALOMA trials have led to approval of palbociclib (Ibrance®) as 1^{st} line therapy in combination with letrozole for HR-positive/HER2-negative metastatic breast cancer in post-menopausal women. Palbociclib was also approved in combination with fulvestrant in 2^{nd} line HR-positive/HER2-negative metastatic breast cancer.
Abemaciclib (Verzenio®), an oral twice daily continuously dosed selective CDK4/6 inhibitor was approved for monotherapy in patients with prior endocrine and chemotherapies, and in combination with fulvestrant for patients that progressed on one prior line of endocrine therapy in a neoadjuvant or adjuvant setting or in first line metastatic breast cancer. Abemaciclib is also approved in combination with an aromatase inhibitor as initial endocrine therapy based on the results of the MONARCH-3 study.
Accordingly, In one embodiment, the present invention provides an IL-1β binding antibody or a functional fragment thereof, suitably gevokizumab or suitably canakinumab for use in combination with a CDK4/6 inhibitor selected from ribociclib, or a pharmaceutically acceptable salt thereof, palbociclib, or a pharmaceutically acceptable salt thereof, and abemaciclib, or a pharmaceutically acceptable salt thereof, in the treatment of hormone receptor (HR)-positive/HER2-negative advanced or metastatic breast cancer. In one embodiment, said breast cancer patient has not received any prior systemic therapy (first line treatment). In another embodiment, said patient has progressed on at least one prior line of endocrine therapy in a neoadjuvant or adjuvant setting.
In one embodiment, the present invention provides an IL-1β binding antibody or a functional fragment thereof, suitably gevokizumab or suitably canakinumab for use in combination with ribociclib, or a pharmaceutically acceptable salt thereof in the treatment of HR positive/HER2 negative positive advanced breast cancer as first and/or second line of endocrine therapy.
In one embodiment, the present invention provides an IL-1β binding antibody or a functional fragment thereof, suitably gevokizumab or suitably canakinumab for use in combination with ribociclib in the treatment of HR positive/HER2 negative positive advanced breast cancer as first and/or second line of endocrine therapy, comprising administering 200 mg of canakinumab or 30 mg to 120 mg gevokizumab every three weeks or every four weeks (monthly) in combination with 200 mg to 600 mg ribociclib, or a pharmaceutically acceptable salt thereof, for 21 consecutive days, followed by 7 days off.
In one embodiment, the present invention provides an IL-1β binding antibody or a functional fragment thereof, suitably gevokizumab or suitably canakinumab for use in combination with ribociclib, or a pharmaceutically acceptable salt thereof in the treatment of HR positive/HER2 negative positive early breast cancer.
In one embodiment, the present invention provides an IL-1β binding antibody or a functional fragment thereof, suitably gevokizumab or suitably canakinumab for use in combination with ribociclib in the treatment of HR positive/HER2 negative positive early breast cancer as first and/or second line of endocrine therapy, comprising administering about 200 mg of canakinumab or about 30 mg to 120 mg gevokizumab every three weeks or every four weeks (monthly) in combination with about 200 mg to 600 mg ribociclib, or a pharmaceutically acceptable salt thereof, for 21 consecutive days, followed by 7 days off.
In one embodiment, the present invention provides an IL-1β binding antibody or a functional fragment thereof, suitably gevokizumab or suitably canakinumab for use in combination with ribociclib, or a pharmaceutically acceptable salt thereof in the treatment of triple negative breast cancer. In one embodiment, the present invention provides an IL-1β binding antibody or a functional fragment thereof, suitably gevokizumab or suitably canakinumab, for use in combination with ribociclib in the treatment of triple negative breast cancer, comprising administering about 200 mg of canakinumab or about 30 mg to about 120 mg gevokizumab every three weeks or every four weeks (monthly) in combination with about 200 mg to about 600 mg ribociclib, or a pharmaceutically acceptable salt thereof, for 21 consecutive days, followed by 7 days off.
In one embodiment, the present invention provides an IL-1β binding antibody or a functional fragment thereof, suitably gevokizumab or suitably canakinumab, for use in combination with ribociclib and aromatase inhibitor in the treatment of hormone receptor (HR)-positive/HER2-negative advanced or metastatic breast cancer, comprising administering about 200 mg of canakinumab or about 30 mg to about 120 mg gevokizumab every three weeks or every four weeks (monthly) in combination with about 200 mg to about 600 mg ribociclib, or a pharmaceutically acceptable salt thereof, for 21 consecutive days, followed by 7 days off and an aromatase inhibitor, preferably letrozole, in accordance with the prescribing information, e.g., 2.5 mg letrozole daily.
In one embodiment, the present invention provides an IL-1β binding antibody or a functional fragment thereof, suitably gevokizumab or suitably canakinumab, for use in combination with ribociclib and letrozole in the treatment of postmenopausal women with hormone receptor positive, HER2-negative, advanced breast cancer who received no prior therapy for advanced disease, comprising administering about 200 mg of canakinumab or about 30 mg to about 120 mg gevokizumab every three weeks or every four weeks (monthly) in combination with about 200 mg to about 600 mg ribociclib, or a pharmaceutically acceptable salt thereof, for 21 consecutive days, followed by 7 days off and letrozole, in accordance with the prescribing information, e.g., 2.5 mg letrozole daily.
In one embodiment, the present invention provides an IL-1β binding antibody or a functional fragment thereof, suitably gevokizumab or suitably canakinumab, for use in combination with ribociclib and letrozole in the treatment of men and pre/postmenopausal women with hormone receptor-positives (HR+) HER2-negative (HER2-) advanced breast cancer (aBC) with no prior hormonal therapy for advanced disease, comprising administering about 200 mg of canakinumab or about 30 mg to about 120 mg gevokizumab every three weeks or every four weeks (monthly) in combination with about 600 mg ribociclib, or a pharmaceutically acceptable salt thereof, for 21 consecutive days, followed by 7 days off and 2.5 mg letrozole daily.
In one embodiment, the present invention provides an IL-1β binding antibody or a functional fragment thereof, suitably gevokizumab or suitably canakinumab, for use in combination with palbociclib in the treatment of hormone receptor (HR)-positive/HER2-negative advanced or metastatic breast cancer, comprising administering about 200 mg of canakinumab or about 30 mg to about 120 mg gevokizumab every three weeks or every four weeks (monthly) in combination with about 75 mg to about 125 mg palbociclib, or a pharmaceutically acceptable salt thereof, for 21 consecutive days, followed by 7 days off and an aromatase inhibitor, preferably letrozole, in accordance with the prescribing information, e.g., 2.5 mg letrozole daily.
In one embodiment, the present invention provides an IL-1β binding antibody or a functional fragment thereof, suitably gevokizumab or suitably canakinumab, for use in combination with abemaciclib in the treatment of hormone receptor (HR)-positive/HER2-negative advanced or metastatic breast cancer, comprising administering about 200 mg of canakinumab or about 30 mg to about 120 mg gevokizumab every three weeks or every four weeks (monthly) in combination with about 50 mg to about 200 mg abemaciclib, or a pharmaceutically acceptable salt thereof, twice daily. Optionally, fulvestrant is additionally administered in accordance with the prescribing information for abemaciclib and fulvestrant.
In one embodiment, the present invention provides an IL-1β binding antibody or a functional fragment thereof, suitably gevokizumab or suitably canakinumab, for use in combination with ribociclib and fulvestrant in the treatment of hormone receptor (HR)-positive/HER2-negative advanced or metastatic breast cancer, comprising administering about 200 mg of canakinumab or about 30 mg to about 120 mg gevokizumab every three weeks or every four weeks (monthly) in combination with 200 mg about to about 600 mg ribociclib, or a pharmaceutically acceptable salt thereof, for 21 consecutive days, followed by 7 days off and 500 mg fulvestrant once every 28 days with 1 additional dose on day 15.
In one embodiment, the present invention provides an IL-1β binding antibody or a functional fragment thereof, suitably gevokizumab or suitably canakinumab, for use in combination with ribociclib and fulvestrant in the treatment of men and postmenopausal women with hormone receptor positive, HER2-negative, advanced breast cancer who have received no or only one line of prior endocrine treatment, comprising administering about 200 mg of canakinumab or about 30 mg to about 120 mg gevokizumab every three weeks or every four weeks (monthly) in combination with about 200 mg to about 600 mg ribociclib, or a pharmaceutically acceptable salt thereof, for 21 consecutive days, followed by 7 days off and 500 mg fulvestrant once every 28 days with 1 additional dose on day 15.
In one embodiment, the present invention provides an IL-1β binding antibody or a functional fragment thereof, suitably gevokizumab or suitably canakinumab, for use in combination with ribociclib, everolimus and exemestane in the treatment of hormone receptor (HR)-positive/HER2-negative locally advanced or metastatic breast cancer. In one embodiment, the present invention provides IL-1β binding antibody or a functional fragment thereof, suitably gevokizumab or suitably canakinumab, for use in combination with ribociclib in the treatment of hormone receptor (HR)-positive/HER2-negative locally advanced or metastatic breast cancer, comprising administering about 200 mg of canakinumab or about 30 mg to about 120 mg gevokizumab every three weeks or every four weeks (monthly) in combination with ribociclib, or a pharmaceutically acceptable salt thereof, everolimus and exemestane, wherein ribociclib, or a pharmaceutically acceptable salt thereof is administered at a dose of about 200 mg to about 600 mg for 21 consecutive days, followed by 7 days off and wherein everolimus and exemestane are administered once daily according to the respective prescribing information.
In one embodiment, the present invention provides an IL-1β binding antibody or a functional fragment thereof, suitably gevokizumab or suitably canakinumab, for use in combination with palbociclib, or a pharmaceutically acceptable salt thereof, and fulvestrant, for use in the treatment of HR+/HER2− advanced/metastatic breast cancer with disease progression following prior endocrine therapy, comprising administering about 200 mg of canakinumab or about 30 mg to about 120 mg gevokizumab every three weeks or every four weeks (monthly) in combination with about 75 mg to about 125 mg palbociclib, or a pharmaceutically acceptable salt thereof, for 21 consecutive days, followed by 7 days off and 500 mg fulvestrant once every 28 days with one additional dose on day 15.
In one embodiment, the present invention provides an IL-1β binding antibody or a functional fragment thereof, suitably gevokizumab or suitably canakinumab, for use in combination with ribociclib and fulvestrant in the treatment of hormone receptor (HR)-positive/HER2-negative advanced or metastatic breast cancer, comprising administering about 200 mg of canakinumab or about 30 mg to about 120 mg gevokizumab every three weeks or every four weeks (monthly) in combination with about 200 mg to about 600 mg ribociclib, or a pharmaceutically acceptable salt thereof, for 21 consecutive days, followed by 7 days off and 500 mg fulvestrant once every 28 days with one additional dose on day 15.
In one embodiment, the present invention provides an IL-1β binding antibody or a functional fragment thereof, suitably gevokizumab or suitably canakinumab for use in combination with ribociclib and letrozole in the treatment of pre-(with goserelin) and postmenopausal women with hormone receptor positive, HER2-negative, advanced breast cancer. In one embodiment, the present invention provides an IL-1β binding antibody or a functional fragment thereof, suitably gevokizumab or suitably canakinumab, for use in combination with ribociclib and letrozole in the treatment of hormone receptor (HR)-positive/HER2-negative locally advanced or metastatic breast cancer, comprising administering about 200 mg of canakinumab or about 30 mg to about 120 mg gevokizumab every three weeks or every four weeks (monthly) in combination with about 200 mg to about 600 mg ribociclib, or a pharmaceutically acceptable salt thereof, and letrozole in accordance with the prescribing information, e.g., about 2.5 mg letrozole daily. In pre-menopausal patients, goserelin is administered additionally.
In one embodiment, the present invention provides an IL-1β binding antibody or a functional fragment thereof, suitably gevokizumab or suitably canakinumab, for use in combination with ribociclib and fulvestrant in the treatment of pre-(with goserelin) and postmenopausal women with hormone receptor positive, HER2-negative, advanced breast cancer. In one embodiment, the present invention provides an IL-1β binding antibody or a functional fragment thereof, suitably gevokizumab or suitably canakinumab, for use in combination with ribociclib and fulvestrant in the treatment of hormone receptor (HR)-positive/HER2-negative locally advanced or metastatic breast cancer, comprising administering about 200 mg of canakinumab or about 30 mg to about 120 mg gevokizumab every three weeks or every four weeks (monthly) in combination with about 200 mg to about 600 mg ribociclib, or a pharmaceutically acceptable salt thereof, and fulvestrant in accordance with the prescribing information, e.g., about 500 mg fulvestrant once every 28 days with 1 additional dose on day 15. In pre-menopausal patients, goserelin is administered additionally.
In one embodiment, the present invention provides an IL-1β binding antibody or a functional fragment thereof, suitably gevokizumab or suitably canakinumab, for use in combination with ribociclib and tamoxifen in the treatment of pre-(with goserelin) and postmenopausal women with hormone receptor positive, HER2-negative, advanced breast cancer. In one embodiment, the present invention provides IL-1β binding antibody or a functional fragment thereof, suitably gevokizumab or suitably canakinumab, for use in combination with ribociclib and fulvestrant in the treatment of hormone receptor (HR)-positive/HER2-negative locally advanced or metastatic breast cancer, comprising administering about 200 mg of canakinumab or about 30 mg to about 120 mg gevokizumab every three weeks or every four weeks (monthly) in combination with about 200 mg to about 600 mg ribociclib, or a pharmaceutically acceptable salt thereof, and tamoxifen in accordance with the prescribing information. In pre-menopausal patients, goserelin is administered additionally.
In one embodiment, the present invention provides an IL-1β binding antibody or a functional fragment thereof, suitably gevokizumab or suitably canakinumab, for use in combination with ribociclib, goserelin and non-steroidal aromatase inhibitor (NSAI) in the treatment of premenopausal women with hormone receptor positive, HER2-negative, advanced breast cancer. In one embodiment, the present invention provides an IL-1β binding antibody or a functional fragment thereof, suitably gevokizumab or suitably canakinumab, for use in combination with ribociclib, goserelin and a non-steroidal aromatase inhibitor (NSAI), suitably selected from anastrazole and letrozole in the treatment of premenopausal women with hormone receptor positive, HER2-negative, advanced breast cancer, comprising administering about 200 mg of canakinumab or about 30 mg to about 120 mg gevokizumab every three weeks or every four weeks (monthly) in combination with about 200 mg to about 600 mg ribociclib, or a pharmaceutically acceptable salt thereof, wherein ribociclib is administered for 21 consecutive days, followed by 7 days off and anastrazole or letrozole are administered in accordance with the prescribing information.
Current standard of care therapeutic agents in first line treatment of metastatic breast cancer therapy are outlined in the 2018 National Comprehensive Cancer Network (NCCN) guidelines for breast cancer (version 3.2018). These include the agents as summarized in.

TABLE 5
Therapeutic regimen and standard of care drugs according
to NCCN guidelines for breast cancer 2018
in the treatment of refractory metastatic breast cancer

| | Therapeutic regimen | Current SoC drugs |
|---|---|---|
| | according to NCCN | according to NCCN |
| Tumor sub- | guidelines for breast | guidelines for breast |
| type | cancer 2018 | cancer 2018 |
| HR+/HER2− | Consider an additional line of | Endocrine: |
| (Luminal A) | endocrine therapy if the patient | Non steroidal |
| | is not refractory or | AI (anastrozole, |
| | chemotherapy (up to 3 | letrozole), steroidal AI |
| | sequential lines) | (exemestane) and |
| | Addition of mTOR inhibitor in | SERM (tamoxifen) |
| | combination with exemestane | SERD: fulvestrant |
| | after 1st line failure with AI | mTOR: everolimus |
| | inhibitors (letrozole/ | PARPi: olaparib, |
| | anastrazole) | talazopanib |
| | PARP inhibitors are approved | |
| | for those with gBRCAm who | |
| | are inappropriate for endocrine | |
| | therapy | |
| HR+/HER2+ | If the patient has progressed on | Alternative regimens |
| (Luminal B) | 1^{st} line endocrine therapy | (to 1^{st} line): |
| | consider an additional line of | T-DM1 |
| | endocrine therapy (if not | Trastuzumab |
| | refractory) +/− HER-2 targeted | with paclitaxel +/− |
| | therapy (up to 3 sequential | carboplatin |
| | endocrine regimens) | Trastuzumab with |
| | If the patient has progressed on | docetaxel |
| | chemotherapy and HER2 | Trastuzumab with |
| | targeted therapy consider | vinorelbine |
| | another line of chemotherapy | Trastuzumab with |
| | and HER-2 targeted therapy | capecitabine |
| | until progression | Lapatinib + |
| | Lapatinib (TKI) and Ado- | capecitabine |
| | trastuzumab emtansine (T- | Trastuzumab + |
| | DM1) are recommended | lapatinib |
| | for use after | |
| | preferred chemotherapy | |
| | regimen 1^{st} line | |
| HR−/HER2+ | If the patient has progressed on | Alternative SoC |
| | chemotherapy and HER2 | regimens are the same |
| | targeted therapy consider | as those described for |
| | another line of chemotherapy | HR+/HER2+ tumors |
| | and HER-2 targeted therapy | |
| | until progression | |
| | Lapatinib (TKI) and Ado- | |
| | trastuzumab emtansine (T- | |
| | DM1) are recommended | |
| | for use after | |
| | preferred chemotherapy | |
| | regimen 1^{st} line | |
| HR−/HER2 | Three sequential lines of | Chemotherapy regimen: |
| (Basal like/ | chemotherapy after progression | No specific |
| TNBC) | PARP inhibitors are approved | recommendations |
| | for gBRCA mutation patients | PARP: olaparib, |
| | in mBC | talazopanib |

In yet another aspect the present invention provides an IL-1β binding antibody or a functional fragment thereof, suitably gevokizumab or suitably canakinumab, in combination with at least one further therapeutic agent in accordance with the therapeutic regimen selected from Table for use in the treatment of refractory metastatic breast cancer.
In one embodiment, the present invention provides an IL-1β binding antibody or a functional fragment thereof, suitably gevokizumab or suitably canakinumab, for use in combination with endocrine therapy in the treatment of breast cancer, wherein breast cancer is hormone receptor (HR)-positive/HER2-negative breast cancer, comprising administering about 200 mg of canakinumab or about 30 mg to about 120 mg gevokizumab every three weeks or every four weeks (monthly) in combination with everolimus and endocrine therapy selected from non-steroidal aromatase inhibitor (anastrazole, letrozole), estrogen receptor antagonist (fulvestrant, NVS-LSZ102, AZD9496, GDC-0927, elacestrant, SAR-439859), SERM (tamoxifen, toremifen) and steroidal aromatase inhibitor (exemestane).
PARP inhibitors inhibit the enzyme poly ADP ribose polymerase (PARP), which is involved in DNA repair. For germline BRCA-mutated (gBRCAm), HER2-negative locally advanced or metastatic breast cancer olarparib (Lynparza®) or talazoparib (Talzenna®) is indicated in patients with deleterious or suspected deleterious gBRCAm, HER2-negative locally advanced or metastatic breast cancer, who have been treated with chemotherapy in the neoadjuvant or adjuvant setting. Patients with hormone receptor (HR)-positive breast cancer should have been treated with a prior endocrine therapy or be considered inappropriate for endocrine therapy. Accordingly, In one embodiment, the present invention provides an IL-1β binding antibody or a functional fragment thereof, suitably gevokizumab or suitably canakinumab, for use in combination with olaparib, or a pharmaceutically acceptable salt thereof, in the treatment of gBRCAm, HER2-negative advanced or metastatic breast cancer. In one embodiment, said patient has progressed on at least one prior line of chemotherapy in a neoadjuvant or adjuvant setting. In one embodiment, the present invention provides an IL-1β binding antibody or a functional fragment thereof, suitably gevokizumab or suitably canakinumab, for use in combination with olaparib in the treatment of gBRCAm, HER2-negative advanced or metastatic breast cancer, comprising administering about 200 mg of canakinumab or about 30 mg to about 120 mg gevokizumab every three weeks or every four weeks (monthly) in combination with olaparib, or a pharmaceutically acceptable salt thereof. In further embodiments, olaparib, or a pharmaceutically acceptable salt thereof, may be administered at an amount of a total daily dose of 400 mg to 600 mg as per the olaparib prescribing information. In one embodiment, the present invention provides an IL-1β binding antibody or a functional fragment thereof, suitably gevokizumab or suitably canakinumab, for use in combination with talazoparib in the treatment of gBRCAm, HER2-negative advanced or metastatic breast cancer, comprising administering about 200 mg of canakinumab or about 30 mg to about 120 mg gevokizumab every three weeks or every four weeks (monthly) in combination with talazoparib, or a pharmaceutically acceptable salt thereof. In further embodiments, talazoparib, or a pharmaceutically acceptable salt thereof, may be administered at an amount of 0.25 mg to 1 mg per day as per the talazoparib prescribing information.
The PI3K/Akt/mTOR pathway is an important, tightly regulated survival pathway for the normal cell. Phosphatidylinositol 3-kinases (PI3Ks) are widely expressed lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃). These products of the PI3K-catalyzed reactions act as second messengers and have central roles in key cellular processes, including cell growth, differentiation, mobility, proliferation and survival. Aberrant regulation of PI3K, which often increases survival through AKT activation, is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110a isoform, PIK3CA, and for AKT are amplified and increased protein expression of their gene products has been demonstrated in several human cancers. Alpelisib and buparlisib have highly selective inhibitory activity for the alpha-isoform of phosphatidylinositol 3-kinase (PI3K). The SOLAR-1 trial demonstrated that alpelisib plus fulvestrant nearly doubled PFS versus fulvestrant alone in men and postmenopausal women with PIK3CA mutated HR+/HER2− advanced breast cancer after progression on an aromatase inhibitor or after receiving up to one additional line of therapy.
Accordingly, In one embodiment, the present invention provides an IL-1β binding antibody or a functional fragment thereof, suitably gevokizumab or suitably canakinumab, for use in combination with alpelisib, or a pharmaceutically acceptable salt thereof, in the treatment of PIK3CA mutated HR+/HER2− advanced breast cancer. In one embodiment, said breast cancer patient has not received any prior systemic therapy (first line treatment). In another embodiment, said patient has progressed on at least one prior line of therapy in a neoadjuvant or adjuvant setting. In one embodiment, the present invention provides an IL-1β binding antibody or a functional fragment thereof, suitably gevokizumab or suitably canakinumab, for use in combination with alpelisib in the treatment of PIK3CA mutated HR+/HER2− advanced breast cancer, comprising administering about 200 mg of canakinumab or about 30 mg to about 120 mg gevokizumab every three weeks or every four weeks (monthly) in combination with alpelisib, or a pharmaceutically acceptable salt thereof, wherein alpelisib is administered by a suitable route, e.g., orally, at an amount of about 50 mg to about 450 mg per day. In further embodiments, alpelisib, or a pharmaceutically acceptable salt thereof, may be administered at an amount of about 200 to about 400 mg per day, or about 240 mg to about 400 mg per day, or about 300 mg to about 400 mg per day, or about 350 mg to about 400 mg per day. In a preferred embodiment, alpelisib, or a pharmaceutically acceptable salt thereof, is administered at an amount of about 350 mg to about 400 mg per day. In another preferred embodiment, alpelisib, or a pharmaceutically acceptable salt thereof, is administered at an amount of about 300 mg per day. Optionally, fulvestrant is additionally administered in accordance with the prescribing information for fulvestrant, e.g., 500 mg intramuscular injections on days 1 and 15 on the first cycle and day 1 of each subsequent 28-day cycle as per fulvestrant prescribing information.
In one embodiment, the present invention provides an IL-1β binding antibody or a functional fragment thereof, suitably gevokizumab or suitably canakinumab, for use in combination with alpelisib, or a pharmaceutically acceptable salt thereof in the treatment of HR-positive/HER2-negative advanced breast cancer as first and/or second line of endocrine therapy.
In one embodiment, the present invention provides an IL-1β binding antibody or a functional fragment thereof, suitably gevokizumab or suitably canakinumab, for use in combination with ribociclib in the treatment of HR-positive/HER2-negative advanced breast cancer as first and/or second line of endocrine therapy, comprising administering about 200 mg of canakinumab or about 30 mg to about 120 mg gevokizumab every three weeks or every four weeks (monthly) in combination with about 200 mg to about 400 mg, preferably 300 mg, alpelisib, or a pharmaceutically acceptable salt thereof.
In one embodiment, the present invention provides an IL-1β binding antibody or a functional fragment thereof, suitably gevokizumab or suitably canakinumab, for use in combination with alpelisib, or a pharmaceutically acceptable salt thereof in the treatment of HR-positive/HER2-negative early breast cancer. In one embodiment, the present invention provides DRUG of the invention for use in combination with ribociclib in the treatment of HR-positive/HER2-negative early breast cancer as first and/or second line of endocrine therapy, comprising administering about 200 mg of canakinumab or about 30 mg to about 120 mg gevokizumab every three weeks or every four weeks (monthly) in combination with about 200 mg to about 400 mg, preferably 300 mg, alpelisib, or a pharmaceutically acceptable salt thereof.
In one embodiment, the present invention provides an IL-1β binding antibody or a functional fragment thereof, suitably gevokizumab or suitably canakinumab, for use in combination with alpelisib, or a pharmaceutically acceptable salt thereof in the treatment of triple negative breast cancer. In one embodiment, the present invention provides an IL-1β binding antibody or a functional fragment thereof, suitably gevokizumab or suitably canakinumab, for use in combination with ribociclib in the treatment of triple negative breast cancer, comprising administering about 200 mg of canakinumab or about 30 mg to about 120 mg gevokizumab every three weeks or every four weeks (monthly) in combination with about 200 mg to about 400 mg, preferably 300 mg, alpelisib, or a pharmaceutically acceptable salt thereof.
In one embodiment, the present invention provides an IL-1β binding antibody or a functional fragment thereof, suitably gevokizumab or suitably canakinumab, for use in combination with alpelisib, or a pharmaceutically acceptable salt thereof, and ribociclib, or a pharmaceutically acceptable salt thereof and letrozole in patients with advanced ER-positive breast cancer. In one embodiment, the present invention provides an IL-1β binding antibody or a functional fragment thereof, suitably gevokizumab or suitably canakinumab, for use in combination with alpelisib in the treatment of advanced ER-positive breast cancer, comprising administering 200 mg of canakinumab or 30 mg to 120 mg gevokizumab every three weeks or every four weeks (monthly) in combination with alpelisib, or a pharmaceutically acceptable salt thereof, wherein alpelisib, or a pharmaceutically acceptable salt thereof is administered at a dose of about 300 mg to 400 mg per day, wherein ribociclib, or a pharmaceutically acceptable salt thereof, is administered at a dose of about 200 mg to 600 mg, for 21 consecutive days, followed by 7 days off and an aromatase inhibitor, preferably letrozole, is administered in accordance with the prescribing information, e.g., 2.5 mg letrozole daily.
In one embodiment, the present invention provides an IL-1β binding antibody or a functional fragment thereof, suitably gevokizumab or suitably canakinumab, for use in combination with ribociclib, or a pharmaceutically acceptable salt thereof, fulvestrant and alpelisib, or a pharmaceutically acceptable salt thereof, in the treatment of postmenopausal women with hormone receptor positive/HER2 negative locally recurrent or advanced metastatic breast cancer. In one embodiment, the present invention provides an IL-1β binding antibody or a functional fragment thereof, suitably gevokizumab or suitably canakinumab, for use in combination with ribociclib, or a pharmaceutically acceptable salt thereof, fulvestrant and alpelisib, or a pharmaceutically acceptable salt thereof, in the treatment of postmenopausal women with hormone receptor positive, HER2 negative locally recurrent or advanced metastatic breast cancer comprising administering about 200 mg of canakinumab or about 30 mg to about 120 mg gevokizumab every three weeks or every four weeks (monthly), alpelisib, or a pharmaceutically acceptable salt thereof, at a dose of about 300 mg to 400 mg per day, ribociclib, or a pharmaceutically acceptable salt thereof, at a dose of about 200 mg to 600 mg, for 21 consecutive days, followed by 7 days off and fulvestrant in accordance with the prescribing information, e.g., 500 mg once monthly.
In one embodiment, the present invention provides an IL-1β binding antibody or a functional fragment thereof, suitably gevokizumab or suitably canakinumab, for use in combination with ribociclib, or a pharmaceutically acceptable salt thereof, fulvestrant and buparlisib, or a pharmaceutically acceptable salt thereof, in the treatment of postmenopausal women with hormone receptor positive, HER2 negative locally recurrent or advanced metastatic breast cancer. In one embodiment, the present invention provides an IL-1β binding antibody or a functional fragment thereof, suitably gevokizumab or suitably canakinumab, for use in combination with ribociclib, or a pharmaceutically acceptable salt thereof, fulvestrant and buparlisib, or a pharmaceutically acceptable salt thereof, in the treatment of postmenopausal women with hormone receptor positive, HER2 negative locally recurrent or advanced metastatic breast cancer comprising administering about 200 mg of canakinumab or about 30 mg to about 120 mg gevokizumab every three weeks or every four weeks (monthly), buparlisib, or a pharmaceutically acceptable salt thereof, ribociclib, or a pharmaceutically acceptable salt thereof, at a dose of about 200 mg to 600 mg, for 21 consecutive days, followed by 7 days off and fulvestrant in accordance with the prescribing information, e.g., 500 mg once monthly.
In one embodiment, the present invention provides an IL-1β binding antibody or a functional fragment thereof, suitably gevokizumab or suitably canakinumab, for use in combination with ribociclib, or a pharmaceutically acceptable salt thereof, letrozole and buparlisib, or a pharmaceutically acceptable salt thereof, in the treatment of HR-positive/HER2-negative post-menopausal women with locally advanced or metastatic breast cancer. In one embodiment, the present invention provides an IL-1β binding antibody or a functional fragment thereof, suitably gevokizumab or suitably canakinumab, for use in combination with ribociclib, or a pharmaceutically acceptable salt thereof, letrozole and buparlisib, or a pharmaceutically acceptable salt thereof, in the treatment of postmenopausal women with HR-positive/HER2-negative locally recurrent or advanced metastatic breast cancer comprising administering about 200 mg of canakinumab or about 30 mg to about 120 mg gevokizumab every three weeks or every four weeks (monthly), buparlisib, or a pharmaceutically acceptable salt thereof, ribociclib, or a pharmaceutically acceptable salt thereof, at a dose of about 200 mg to 600 mg, for 21 consecutive days, followed by 7 days off and letrozole in accordance with the prescribing information, e.g., 2.5 mg daily.
In one embodiment, the present invention provides an IL-1β binding antibody or a functional fragment thereof, suitably gevokizumab or suitably canakinumab, for use in combination with ribociclib, or a pharmaceutically acceptable salt thereof, everolimus and exemestane, in the treatment of men and postmenopausal women with HR-positive/HER2-negative locally advanced or metastatic breast cancer following progression on a CDK 4/6 inhibitor. In one embodiment, the present invention provides an IL-1β binding antibody or a functional fragment thereof, suitably gevokizumab or suitably canakinumab, for use in combination with ribociclib, or a pharmaceutically acceptable salt thereof, everolimus and exemestane in the treatment of men and postmenopausal women with HR-positive/HER2-negative locally advanced or metastatic breast cancer comprising administering about 200 mg of canakinumab or about 30 mg to about 120 mg gevokizumab every three weeks or every four weeks (monthly), ribociclib, or a pharmaceutically acceptable salt thereof, at a dose of about 200 mg to 600 mg, for 21 consecutive days, followed by 7 days off, everolimus in accordance with the prescribing information, e.g., 10 mg per day, and exemestane in accordance with the prescribing information, e.g., 25 mg per day.
In one embodiment, the present invention provides an IL-1β binding antibody or a functional fragment thereof, suitably gevokizumab or suitably canakinumab, for use in combination with NVS-LSZ102, or a pharmaceutically acceptable salt thereof, in patients with advanced or metastatic ER-positive breast cancer who have progressed after endocrine therapy. In one embodiment, the present invention provides an IL-1β binding antibody or a functional fragment thereof, suitably gevokizumab or suitably canakinumab, for use in combination with NVS-LSZ102, or a pharmaceutically acceptable salt thereof, and buparlisib, or a pharmaceutically acceptable salt thereof, in patients with advanced or metastatic ER-positive breast cancer who have progressed after endocrine therapy. In one embodiment, the present invention provides an IL-1β binding antibody or a functional fragment thereof, suitably gevokizumab or suitably canakinumab, for use in combination with NVS-LSZ102, or a pharmaceutically acceptable salt thereof, and buparlisib, or a pharmaceutically acceptable salt thereof, in patients with advanced or metastatic ER-positive breast cancer who have progressed after endocrine therapy, comprising administering about 200 mg of canakinumab or about 30 mg to about 120 mg gevokizumab every three weeks or every four weeks (monthly), NVS-LSZ102 once daily and buparlisib, or a pharmaceutically acceptable salt thereof.
In one embodiment, the present invention provides an IL-1β binding antibody or a functional fragment thereof, suitably gevokizumab or suitably canakinumab, for use in combination with NVS-LSZ102, or a pharmaceutically acceptable salt thereof, and alpelisib, or a pharmaceutically acceptable salt thereof, in patients with advanced or metastatic ER-positive breast cancer who have progressed after endocrine therapy. In one embodiment, the present invention provides an IL-1β binding antibody or a functional fragment thereof, suitably gevokizumab or suitably canakinumab, for use in combination with NVS-LSZ102, or a pharmaceutically acceptable salt thereof, and alpelisib, or a pharmaceutically acceptable salt thereof, in patients with advanced or metastatic ER-positive breast cancer who have progressed after endocrine therapy, comprising administering about 200 mg of canakinumab or about 30 mg to about 120 mg gevokizumab every three weeks or every four weeks (monthly), NVS-LSZ102 once daily and alpelisib, or a pharmaceutically acceptable salt thereof at a dose of about 300 mg to 400 mg per day.

### Glioblastoma

In one aspect the present invention provides an IL-1β binding antibody or a functional fragment thereof, suitably gevokizumab or suitably canakinumab, alone or in combination with one or more therapeutic agents, for use in the treatment of cancer, e.g., cancer having at least partial inflammatory basis, wherein said cancer is glioblastoma.

Glioblastoma is an aggressive type of cancer that can occur in the brain or spinal cord. Glioblastoma forms from cells called astrocytes that support nerve cells.

In one embodiment, the present invention provides DRUG of the invention, preferably canakinumab or gevokizumab, alone or preferably in combination with one or more therapeutic agents, for use in the treatment of metastatic glioblastoma.

In one embodiment, the present invention provides DRUG of the invention, preferably canakinumab or gevokizumab, for use in the treatment of pancreatic cancer, wherein DRUG of the invention is administered in combination with one or more therapeutic agents, e.g., a chemotherapeutic agent, a targeted therapy agent, a checkpoint inhibitor or a combination of these agents. In one embodiment DRUG of the invention is administered in combination with a radiotherapy.

In one embodiment, the present invention provides DRUG of the invention, suitably gevokizumab or canakinumab, for use in the treatment of glioblastoma, in combination with one or more therapeutic agents, e.g., chemotherapeutic agent or e.g., a check point inhibitor. In one embodiment, the therapeutic agent, e.g., chemotherapeutic agent, is the standard of care agent for glioblastoma. In one embodiment, the standard of care agent is temozolomide and/or bevacizumab. In one embodiment, the one or more therapeutic agents is selected from a group consisting of temozolomide, bevacizumab, pembrolizumab and nivolumab. DRUG of the invention is administered in combination with Depending on the patient condition, one, two or three therapeutic agents can be selected from the list above, to be combined with gevokizumab or canakinumab.

Chronic inflammation and IL-1β have been associated with a poor histological response to neo-adjuvant therapy and risk of developing cancer (Delitto et al., BMC cancer. 2015), potentially supporting use of DRUG of the invention, preferably canakinumab or gevokizumab, in the neo-adjuvant setting when used in combination with existing SoC adjuvant treatment. Thus In one embodiment, the present invention provides DRUG of the invention, preferably canakinumab or gevokizumab, for use in the neo-adjuvant treatment.

In one embodiment, the present invention provides DRUG of the invention, preferably canakinumab or gevokizumab, for use in the neo-adjuvant treatment. Often SoC drug in the neo-adjuvant treatment is the same drug as adjuvant treatment. In one embodiment, the present invention provides DRUG of the invention, preferably canakinumab or gevokizumab, for use in the prevention of recurrence or relapse of glioblastoma, which has been surgically removed (adjuvant treatment).

In one embodiment, DRUG of the invention is used as monotherapy in the adjuvant treatment. This is preferred due to the good safety profile of canakinumab or gevokizumab.

DRUG of the invention, preferably canakinumab or gevokizumab, is suited for use as adjuvant treatment. In one embodiment, DRUG of the invention is used, in combination with one or more therapeutic agents, in the adjuvant treatment.

In one embodiment, one or more therapeutic agents is the SoC in the glioblastoma adjuvant treatment. Often SoC drug in the neo-adjuvant treatment is the same drug as adjuvant treatment. Often the SoC drug in the adjuvant treatment is the same drug as SoC in the first line treatment.

In one embodiment, DRUG of the invention is used as monotherapy in the glioblastoma adjuvant treatment after the patient has received at least 2 cycles, at least 4 cycles or has completed the intended chemotherapy as adjuvant treatment, suitably the intended chemotherapy is temozolomide and/or bevacizumab.

In one embodiment, DRUG of the invention is used in glioblastoma treatment in combination at the same time as chemotherapy, suitably the intended chemotherapy is temozolomide and/or bevacizumab.

In one embodiment, DRUG of the invention, preferably canakinumab or gevokizumab is used alone or preferably in combination with one or more therapeutic agents, in the first line treatment of pancreatic cancer. In one embodiment, the one or more therapeutic agents is a therapeutic agent used as first line treatment selected from temozolomide and bevacizumab.

In one embodiment, DRUG of the invention, preferably canakinumab or gevokizumab, is used, alone or preferably in combination with one or more therapeutic agents, in second or third line treatment of glioblastoma. In one embodiment, one or more therapeutic agents is selected from temozolomide, bevacizumab, pembrolizumab and nivolumab.

In one embodiment, the treatment, e.g., the adjuvant treatment, the first line treatment or the 2^{nd} or 3^{rd} line treatment continues until disease progress, preferably according to RECIST 1.1.

All the uses disclosed throughout this application, including but not limited to, doses and dosing regimens, combinations, route of administration and biomarkers can be applied to the treatment of glioblastoma.

### Pancreatic

In one aspect the present invention provides an IL-1β binding antibody or a functional fragment thereof, suitably gevokizumab or suitably canakinumab, alone or in combination with one or more therapeutic agents, for use in the treatment of cancer, e.g., cancer having at least partial inflammatory basis, wherein said cancer is pancreatic cancer.

As used herein, the term “pancreatic cancer” refers to pancreatic exocrine tumors and neuroendocrine cancers. This is based on the cell type they start in. About 95% of pancreatic cancers are exocrine tumors, including adenocarcinoma, specifically pancreatic ducal adenocarcinoma (PD C), which is the most common solid tumor type in the pancreas accounting for 80% of cases of pancreatic cancer; acinar cell carcinoma; intraductal papillary-mucinous neoplasm; and mucinous cystadenocarcinoma. Pancreatic neuroendocrine tumors are classified by the hormones they produce. Common types are: gastrinoma (gastrin), glucaganoma (glucagon), insulinoma (insulin), somatostatinoma (somatostatin) VIPoma (vasoactive intestinal peptide), nonfunctional islet cell tumor (no hormones). In one preferred embodiment the cancer is PDAC.

There are a number of observations showing that IL-1β plays a role in pancreatic cancers. In PDAC patients, circulating levels of IL-1β are consistently increased across multiple studies (Yako et al., PLoS One. 2016). It has also been found that functional pro-inflammatory genotypes within the IL-1β gene are associated with risk of pancreatic cancer and its prognosis (Hamacher et al., Cytokine. 2009).

According to the stage of cancer progression, the term “pancreatic cancer” includes primary pancreatic cancer, locally advanced pancreatic cancer, unresectable pancreatic cancer, metastatic pancreatic cancer, refractory pancreatic cancer, and/or cancer drug resistant pancreatic cancer. In one embodiment, the present invention provides DRUG of the invention, preferably canakinumab or gevokizumab, alone or preferably in combination with one or more therapeutic agents, for use in the treatment of metastatic pancreatic cancer.

In one embodiment, the present invention provides DRUG of the invention, preferably canakinumab or gevokizumab, for use in the treatment of pancreatic cancer, wherein DRUG of the invention is administered in combination with one or more therapeutic agents, e.g., a chemotherapeutic agent, a targeted therapy agent, a checkpoint inhibitor or a combination of these agents.

In one embodiment, the present invention provides DRUG of the invention, suitably gevokizumab or canakinumab, for use in the treatment of pancreatic cancer, in combination with one or more therapeutic agents, e.g., chemotherapeutic agent or e.g., a check point inhibitor. In one embodiment, the therapeutic agent, e.g., chemotherapeutic agent, is the standard of care agent for pancreatic cancer. In one embodiment, the one or more therapeutic agents, e.g., chemotherapeutic agent, is selected from nab-paclitaxel (Paclitaxel Albumin-stabilized Nanoparticle Formulation; Abraxane®), docetaxel, capecitabine, erlotinib hydrochloride (Tarceva®), sunitinib malate (Sutent®), fluorouracil (5-FU), gemcitabine hydrochloride, irinotecan, mitomycin C, FOLFIRINOX (leucovorin calcium (folinic acid), fluorouracil, irinotecan hydrochloride and oxaliplatin), gemcitabine plus cisplatin, gemcitabine plus oxaliplatin, gemcitabine plus nab-paclitaxel, and OFF (oxaliplatin, fluorouracil and leucovorin calcium (folinic acid). Depending on the patient condition, one, two or three therapeutic agents can be selected from the list above, to be combined with gevokizumab or canakinumab.

In one embodiment, the one or more therapeutic agents is selected from capecitabine, CI 5-FU, gemcitabine, FOLFIRI, FOLFOX, FOLFIRINOX, modified FOLFIRINOX, OFF, leucovorin, albumin bound paclitaxel, cisplatin, liposomal irinotecan, capecitabine, oxaliplatin, erlotinib, sunitinib, everolimus, pembrolizumab, nivolumab, spartalizumab, atezolizumab, avelumab, ipilimumab, and durvalumab. Depending on the patient condition, one, two, three, or four of the therapeutic agents can be selected from the above lists to be combined with DRUG of the invention.

In one embodiment, the one or more therapeutic agents is the standard of care (SoC) agent for pancreatic cancer. In one preferred embodiment the one or more therapeutic agents is pembrolizumab. In one preferred embodiment the one or more therapeutic agents is erlotinib. In one preferred embodiment the one or more therapeutic agents is sunitinib. In one embodiment, the one or more therapeutic agents is gemcitabine. Existing evidence has demonstrated that IL-1β is implicated in resistance to gemcitabine therapy (Zhang et al., Cancer Res. 2018; 78(7):1700-1712), offering the opportunity for DRUG of the invention, preferably canakinumab or gevokizumab, to be combined with gemcitabine based chemotherapeutic regimens.

In one embodiment, DRUG of the invention is used in the pancreatic cancer treatment in combination with one or more therapeutic agents, further in combination with a radiation therapy. In one preferred embodiment DRUG of the invention is used in the pancreatic cancer treatment in combination with one or more therapeutic agents selected from capecitabine or CI 5-FU or gemcitabine, in combination with a radiation therapy.

Chronic inflammation and IL-1β have been associated with a poor histological response to neo-adjuvant therapy and risk of developing cancer (Delitto et al., BMC cancer. 2015), potentially supporting use of DRUG of the invention, preferably canakinumab or gevokizumab, in the neo-adjuvant setting when used in combination with existing SoC adjuvant treatment. Thus In one embodiment, the present invention provides DRUG of the invention, preferably canakinumab or gevokizumab, for use in the neo-adjuvant treatment.

In one embodiment, the present invention provides DRUG of the invention, preferably canakinumab or gevokizumab, for use in the neo-adjuvant treatment. Often SoC drug in the neo-adjuvant treatment is the same drug as adjuvant treatment. In one embodiment, the present invention provides DRUG of the invention, preferably canakinumab or gevokizumab, for use in the prevention of recurrence or relapse of pancreatic cancer, which has been surgically removed (adjuvant treatment).

In one embodiment, DRUG of the invention is used as monotherapy in the adjuvant treatment. This is preferred due to the good safety profile of canakinumab or gevokizumab.

DRUG of the invention, preferably canakinumab or gevokizumab, is suited for use as adjuvant treatment. In one embodiment, DRUG of the invention is used, in combination with one or more therapeutic agents, in the adjuvant treatment.

In one embodiment, one or more therapeutic agents is the SoC in the pancreatic cancer adjuvant treatment. Often SoC drug in the neo-adjuvant treatment is the same drug as adjuvant treatment. Often the SoC drug in the adjuvant treatment is the same drug as SoC in the first line treatment. The SoC in the adjuvant treatment is gemcitabine+capecitabine or modified FOLFIRINOX. Other recommended regimens are gemcitabine or 5-FU/leucovorin.

In one embodiment, DRUG of the invention is used as monotherapy in the pancreatic cancer adjuvant treatment after the patient has received at least 2 cycles, at least 4 cycles or has completed the intended chemotherapy as adjuvant treatment, suitably the intended chemotherapy is gemcitabine+capecitabine or modified FOLFIRINOX.

In one embodiment, DRUG of the invention is used in the pancreatic cancer adjuvant treatment in combination at the same time as chemotherapy, suitably the intended chemotherapy is gemcitabine+capecitabine or modified FOLFIRINOX.

In one embodiment, DRUG of the invention, preferably canakinumab or gevokizumab is used alone or preferably in combination with one or more therapeutic agents, in the first line treatment of pancreatic cancer. In one embodiment, the one or more therapeutic agents is a therapeutic agent used as first line treatment selected from FOLFIRINOX, modified FOLFIRINOX, gemcitabine+albumin bound paclitaxel, erlotinib+gemcitabine, capecitabine, or CI 5-FU. For BRCA1/2 or PALB mutations, the one or more therapeutic agents used as first line treatment is selected from FOLFIRINOX or gemcitabine+cisplatin.

Preferably DRUG of the invention is used in combination with one or more therapeutic agents, such as the SoC drugs, which are approved as the first line treatment of pancreatic cancer, for example FOLFIRINOX, modified FOLFIRINOX, gemcitabine+albumin bound paclitaxel, erlotinib+gemcitabine, capecitabine, CI 5-FU, or gemcitabine+cisplatin.

In one embodiment, DRUG of the invention, preferably canakinumab or gevokizumab, is used, alone or preferably in combination with one or more therapeutic agents, in second or third line treatment of pancreatic cancer. In one embodiment, one or more therapeutic agents is selected from 5-FU+ leucovorin+liposomal irinotecan, FOLFIRI, FOLFIRINOX, OFF, FOLFOX, capecitabine/oxaliplatin, capecitabine, and CI 5-FU for prior gemcitabine treated patients. In one embodiment, one or more therapeutic agents is selected from gemcitabine, gemcitabine+paclitaxel, gemcitabine+cisplatin (for BRCA1/2 or PALB2), gemcitabine+erlotinib, and 5-FU+leucovorin+liposomal irinotecan for prior fluoropyrimidine treated patients. In one embodiment, one or more therapeutic agents is selected from gemcitabine or capecitabine or CI 5-FU for patients with poor performance status.

In one embodiment, the treatment, e.g., the adjuvant treatment, the first line treatment or the 2^{nd} or 3^{rd} line treatment continues until disease progress, preferably according to RECIST 1.1.

In one embodiment, the one or more therapeutic agents is the combination of albumin bound paclitaxel, e.g., Abraxane®, gemcitabine (“PanCan triple combo”). In one embodiment, the one or more therapeutic agents is the combination of albumin bound paclitaxel, e.g., Abraxane®, gemcitabine and spartalizumab (“PanCan quadrople combo”). In one embodiment, the pancreatic cancer is metastatic pancreatic adenocarcinoma, suitably confirmed histologically or cytologically. In one embodiment, the p In one embodiment, the pancreatic cancer is first line metastatic pancreatic adenocarcinoma. the IL-1β binding antibody is canakinumab. In one embodiment, the dose regimen is 250 mg, every 4 weeks. In one embodiment, canakinumab is administered subcutaneously.

In one embodiment, canakinumab is administered on the same day as spartalizumab, suitably spartalizumab is administered IV 400 mg every 4 weeks. In one embodiment canakinumab, with or without spartalizumab, is administered on top of the standard of care. In one embodiment the standard of care is albumin bound paclitaxel, e.g., Abraxane®, and gemcitabine. Suitably the SoC is gemcitabine 1000 mg/m2+Abraxane at 125 mg/m² IV on day 1, 8, 15 of a 28 day cycle (“PanCan SoC”).

In one embodiment, the overall survival (OS) period of patients receiving treatment of PanCan quadrople combo is extended by at least 2 months, at least 3 months, suitably 3 months, at least 6 months, suitably 6 months, preferably compared to patients receiving treatment of PanCan SoC. In one embodiment, OS is extended by at least 6 months, suitably 6 months, suitably 12 months in the first line treatment settings.

In one embodiment, patient receiving treatment of PanCan quadrople combo has at least 6 months, suitably 6 months, at least 12 months, suitably 12 months overall survival.

In one embodiment, the progression free survival (PFS) period of patients receiving treatment of PanCan quadrople combo is extended by at least 2 months, at least 3 months, suitably 3 months, at least 6 months, suitably 6 months, preferably compared to patients receiving treatment of PanCan SoC. In one embodiment, OS is extended by at least 6 months, suitably 6 months, suitably 12 months in the first line treatment settings.

In one embodiment, patient receiving treatment of PanCan quadrople combo has at least 6 months, suitably 6 months, at least 12 months, suitably 12 months progression free survival.

All the uses disclosed throughout this application, including but not limited to, doses and dosing regimens, combinations, route of administration and biomarkers can be applied to the treatment of pancreatic cancer.

### Head, Neck and Oral Cancer

In one aspect the present invention provides an IL-1β binding antibody or a functional fragment thereof, suitably gevokizumab or suitably canakinumab, alone or in combination with one or more therapeutic agents, for use in the treatment of cancer, e.g., cancer having at least partial inflammatory basis, wherein said cancer is head and neck cancer (HNC) including oral cancer, including HPV, EBV and tobacco and/or alcohol and/or betel quid induced head and neck cancer. Head and neck cancers are further categorized by the area of the head or neck in which they begin. The term “head and neck cancer” or “HNC” as used herein refers to oral cavity cancer (also referred to as oral cancer), nasopharynx cancer (including lymphoepithelioma), oropharynx cancer, hypopharynx cancer, larynx cancer, paranasal sinuses cancer, nasal cavity cancer, salivary gland cancer, head and neck sarcoma, or head and neck lymphoma. In 95% of the cases, head and neck cancers begin in the squamous cells that line the moist, mucosal surfaces inside the head and neck. These squamous cell cancers are often referred to as squamous cell carcinomas of the head and neck. Head and neck cancers can also begin in the salivary glands, but salivary gland cancers are relatively uncommon. There are also head and neck sarcomas, which are rare tumors, accounting for only 1% of all head and neck malignancies. Further, there are head and neck lymphomas. Head and neck are the second most common regions for extra-nodal lymphomas. In one embodiment, the head and neck cancer is oral cancer, e.g., oral squamous cell carcinoma (OSCC).

There are a number of observations showing that IL-1β plays a role in oral cancer. Saliva protein levels of IL-1β are consistently elevated in patients with OSCC, while changes to the IL-1β gene (single nucleotide polymorphisms, SNPs) are associated with risk of developing oral cancer (Netto et al., Clin Cancer Res. 2016; Kamatani et al., Cytokine. 2013; Lakanpal et al., Cancer Genet. 2014). IL-1β is up-regulated by exposure to common oral carcinogens such as tobacco and betel quid, and contributes to malignant transformation and tumor aggressiveness via the promotion of angiogenic and EMT pathways (Lee et al., J Cell Physiol. 2015). Further, up-regulation of IL-1β (alongside the NLRP3 inflammasome) has also been implicated in 5-FU chemotherapy resistance (Feng et al., J Exp Clin Cancer Res. 2017).

According to the stage of cancer progression, the term “head and neck cancer” or “HNC” includes primary I-INC, e.g., primary oral cancer, locally advanced HNC, e.g. locally advanced oral cancer, unresectable HNC, e.g., unresectable oral cancer, metastatic HNC, e.g., metastatic oral cancer, refractory HNC, e.g., refractory oral cancer, and/or cancer drug resistant HNC, e.g., cancer drug resistant oral cancer.

In one embodiment, the present invention provides DRUG of the invention, preferably canakinumab or gevokizumab, alone or preferably in combination with one or more therapeutic agents, for use in the treatment of metastatic HNC, e.g., oral cancer.

In one embodiment, the present invention provides DRUG of the invention, preferably canakinumab or gevokizumab, for use in the treatment of HNC, e.g., oral cancer, wherein DRUG of the invention is administered in combination with one or more therapeutic agents, e.g., a chemotherapeutic agent, a targeted therapy agent, a checkpoint inhibitor or a combination of these agents.

In one embodiment, the one or more therapeutic agents is a chemotherapeutic agent, e.g., selected from platinums, fluorouracil (5-FU), cetuximab, taxanes, bleomycin, ifosfamide, vinblastine, gemcitabine, navelbine, iressa, tarceva, BIBW, paclitaxel, docetaxel, capecitabine, and methotrexate. In one embodiment, the one or more chemotherapeutic agents is alpelisib. Alpelisib is administered at a therapeutically effective amount of about 300 mg per day. In one embodiment, one or more therapeutic agents is a targeted therapy agent selected from EGFR inhibitors, e.g., antibodies, e.g., panitumumab and cetuximab, or tyrosine kinase inhibitors, e.g., afatinib, erlotinib, gefitinib, and lapatinib; VEGF inhibitors e.g., antibodies, e.g., bevacizumab, ranibizumab, or VEGFR inhibitors, e.g., lapatinib, sunitinib, sorafenib, axitinib and pazopanib; mTOR Inhibitors, e.g., everolimus; or MET or HGF inhibitors. In one embodiment, the one or more therapeutic agent is a checkpoint inhibitor, selected from PD-1 inhibitors, e.g., pembrolizumab, nivolumab, spartalizumab (PDR-001); PD-L1 inhibitors, e.g., atezolizumab, avelumab; CTLA-4 inhibitors, e.g., ipilimumab; or other immune modulators, e.g., durvalumab. Depending on the patient condition, one, two or three of the therapeutic agents can be selected from the above lists to be combined with DRUG of the invention.

In one embodiment, the present invention provides DRUG of the invention, preferably canakinumab or gevokizumab, for use in the treatment of HNC, e.g. oral cancer, wherein DRUG of the invention is administered in combination with a combination of one or more chemotherapeutic agents and one or more targeted therapy agents, a combination of one or more chemotherapeutic agents and one or more checkpoint inhibitors, a combination of one or more chemotherapeutic agents and one or more targeted therapy agents and one or more checkpoint inhibitors.

In one preferred embodiment the therapeutic agent is pembrolizumab. In one preferred embodiment the therapeutic agent is nivolumab. In one embodiment, the one or more therapeutic agents is a combination of a platinums, fluorouracil (5-FU), and cetuximab. In one embodiment, the one or more therapeutic agents is the standard of care (SoC) agent for INC, e.g., oral cancer.

In one embodiment, DRUG of the invention is used in the HNC, e.g. oral cancer, treatment in combination with one or more therapeutic agents, further in combination with a radiation therapy.

In one embodiment, the present invention provides DRUG of the invention, preferably canakinumab or gevokizumab, for use in the neo-adjuvant treatment. In one embodiment, the present invention provides DRUG of the invention, preferably canakinumab or gevokizumab, for use in the prevention of recurrence or relapse of HNC, e.g., oral cancer, which has been surgically removed (adjuvant treatment). In one embodiment, DRUG of the invention is used, in combination of one or more therapeutic agents, in the adjuvant treatment. In one embodiment, one or more therapeutic agent is the SoC in the HNC, e.g., oral cancer, adjuvant treatment. Often SoC drug in the neo-adjuvant treatment is the same drug as adjuvant treatment. Often the SoC drug in the adjuvant treatment is the same drug as SoC in the first line treatment.

In one embodiment, DRUG of the invention is used as monotherapy in the adjuvant treatment. This is preferred due to the good safety profile of canakinumab or gevokizumab.

SoC of high risk of relapse head and neck squamous cell carcinoma after surgical resection are chemotherapy e.g., with a platinum, with or without radiation therapy. Known risk factors for recurrence are: microscopic resection margin-positive, extracapsular nodal extension-positive, multiple cervical lymph node metastasis (≥2), lymph node metastasis with a diameter of 3 cm or more, perineural invasion, Level 4 (inferior internal jugular lymph node) or Level 5 (accessory nerve lymph node) lymph node metastasis in oropharyngeal cancer/oral cavity cancer and signs of vascular tumor embolism.

In one embodiment, DRUG of the invention is used as monotherapy in the HNC, e.g., oral cancer, adjuvant treatment after the patient has received radiation therapy and/or at least 2 cycles, at least 4 cycles or has completed the intended chemotherapy as adjuvant treatment, suitably the intended chemotherapy is platinium+5-FU+cetuximab.

In one embodiment, DRUG of the invention is used in the HNC, e.g., oral cancer, adjuvant treatment in combination at the same time as radiation therapy and/or chemotherapy, suitably the intended chemotherapy is platinium+5-FU+cetuximab.

In one embodiment, DRUG of the invention, preferably canakinumab or gevokizumab is used, alone or preferably in combination with one or more therapeutic agents, in the first line treatment of HNC, e.g., oral cancer. In one embodiment, the one or more therapeutic agents is a therapeutic agent used as first line treatment selected from platinums, fluorouracil (5-FU), cetuximab, taxanes, bleomycin, ifosfamide, vinblastine, gemcitabine, navelbine, iressa, tarceva, BIBW, pembrolizumab, and nivolumab. In one embodiment, the one or more therapeutic agent is platinums, fluorouracil (5-FU), and cetuximab. In one embodiment, the one or more therapeutic agent is pembrolizumab. In one embodiment, the one or more therapeutic agent is nivolumab.

In one embodiment, DRUG of the invention is used as monotherapy in the adjuvant treatment. This is preferred due to the good safety profile of canakinumab or gevokizumab.

Preferably DRUG of the invention is used in combination with one or more therapeutic agents with the SoC drugs, which are approved as the first line treatment of HNC, e.g., oral cancer, for example platinium+5-FU+cetuximab.

In one embodiment, DRUG of the invention, preferably canakinumab or gevokizumab is used, alone or preferably in combination with one or more therapeutic agent, in second or third line treatment of HNC, e.g., oral cancer. In one embodiment, one or more therapeutic agents is selected from paclitaxel, docetaxel, and methotrexate. In one embodiment, one or more therapeutic agents is selected from pembrolizumab and nivolumab.

In one embodiment, the treatment, e.g., the adjuvant treatment, the first line treatment or the 2^{nd} or 3^{rd} line treatment continues until disease progress, preferably according to RECIST 1.1.

All the uses disclosed throughout this application, including but not limited to, doses and dosing regimens, combinations, route of administration and biomarkers can be applied to the treatment of HNC, e.g., oral cancer.

The word “a” and “an” have been generally defined as “at least one” or “one or more” in the specification.

The word “patient” refers to human patient.

Unless otherwise specifically stated or clear from context, as used herein, the term “about” in relation to a numerical value is understood as being within the normal tolerance in the art, e.g., within two standard deviations of the mean. Thus, “about” can be within +/−10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The following Examples illustrate the invention described above; they are not, however, intended to limit the scope of the invention in any way.

### EXAMPLE

The Example below is set forth to aid in the understanding of the invention but is not intended, and should not be construed, to limit its scope in any way.

### Example 1

### Tumor-Derived IL-1β Induces Differential Tumor Promoting Mechanisms in Metastasis

### Materials and Methods

### Cell Culture

Human breast cancer MDA-MB-231-Luc2-TdTomato (Calliper Life Sciences, Manchester UK), MDA-MB-231 (parental) MCF7, T47D (European Collection of Authenticated Cell Cultures (ECACC)), MDA-MB-231-IV (Nutter et al., 2014) as well as bone marrow HS5 (ECACC) and human primary osteoblasts OB1 were cultured in DMEM+10% FCS (Gibco, Invitrogen, Paisley, UK). All cell lines were cultured in a humidified incubator under 5% C02 and used at low passage >20.

### Transfection of Tumor Cells

Human MDA-MB-231, MCF 7 and T47D cells were stably transfected to overexpress genes IL1B or IL1R1 using plasmid DNA purified from competent E. coli that were transduced with an ORF plasmid containing human IL1B or IL1R1 (Accession numbers NM_000576 and NM_0008777.2, respectively) with a C-terminal GFP tag (OnGene Technologies Inc. Rockville Md.). Plasmid DNA purification was performed using a PureLink™ HiPure Plasmid Miniprep Kit (ThemoFisher) and DNA quantified by UV spectroscopy before being introduced into human cells with the aid of Lipofectamine II (ThermoFisher). Control cells were transfected with DNA isolated from the same plasmid without IL-1β or IL-1R1 encoding sequences.

### In Vitro Studies

In vitro studies were carried out with and without addition of 0-5 ng/ml recombinant IL-1β (R&D systems, Wiesbaden, Germany)+/−50 μM IL-1Ra (Amgen, Cambridge, UK).
Cells were transferred into fresh media with 10% or 1% FCS. Cell proliferation was monitored every 24 h for up to 120 h by manual cell counting using a 1/400 mm² hemocytometer (Hawkley, Lancing UK) or over a 72 h period using an Xcelligence RTCA DP Instrument (Acea Biosciences, Inc). Tumor cell invasion was assessed using 6 mm transwell plates with an 8 μm pore size (Corning Inc) with or without basement membrane (20% Matrigel; Invitrogen). Tumor cells were seeded into the inner chamber at a density of 2.5×10⁵ for parental as well as MDA-MB-231 derivatives and 5×10⁵ for T47D in DMEM+1% FCS and 5×10⁵ OB1 osteoblast cells supplemented with 5% FCS were added to the outer chamber. Cells were removed from the top surface of the membrane 24 h and 48 h after seeding and cells that had invaded through the pores were stained with hematoxylin and eosin (H&E) before being imaged on a Leica DM7900 light microscope and manually counted.
Migration of cells was investigated by analyzing wound closure: Cells were seeded onto 0.2% gelatine in 6-well tissue culture plates (Costar; Corning, Inc) and, once confluent, 10 μg/ml mitomycin C was added to inhibit cell proliferation and a 50 μm scratch made across the monolayer. The percentage of wound closure was measured at 24 h and 48 h using a CTR7000 inverted microscope and LAS-AF v2.1.1 software (Leica Applications Suite; Leica Microsystems, Wetzlar, Germany). All proliferation, invasion and migration experiments were repeated using the Xcelligence RTCA DP instrument and RCTA Software (Acea Biosystems, Inc).
For co-culture studies with human bone 5×10⁵ MDA-MB-231 or T47D cells were seeded onto tissue culture plastic or into 0.5 cm³ human bone discs for 24 h. Media was removed and analysed for concentration of IL-1β by ELISA. For co-culture with HS5 or OB1 cells, 1×10⁵ MDA-MB-231 or T47D cells were cultured onto plastic along with 2×10⁵ HS5 or OB1 cells. Cells were sorted by FACS 24 h later and counted and lysed for analysis of IL-1β concentration. Cells were collected, sorted and counted every 24 h for 120 h.

### Animals

Experiments using human bone grafts were carried out in 10-week old female NOD SCID mice. In IL-1β/IL-1R1 overexpression bone homing experiments 6 to 8-week old female BALB/c nude mice were used. To investigate effects of IL-1β on the bone microenvironment 10-week old female C57BL/6 mice (Charles River, Kent, UK) or IL-1R1^{-/-} mice (Abdulaal et al., 2016) were used. Mice were maintained on a 12 h: 12 h light/dark cycle with free access to food and water. Experiments were carried out with UK home office approval under project licence 40/3531, University of Sheffield, UK.

### Patient Consent and Preparation of Bone Discs

All patients provided written, informed consent prior to participation in this study. Human bone samples were collected under HTA licence 12182, Sheffield Musculoskeletal Biobank, University of Sheffield, UK. Trabecular bone cores were prepared from the femoral heads of female patients undergoing hip replacement surgery using an Isomat 4000 Precision saw (Buehler) with Precision diamond wafering blade (Buehler). 5 mm diameter discs were subsequently cut using a bone trephine before storing in sterile PBS at ambient temperature.

### In Vivo Studies

To model human breast cancer metastasis to human bone implants two human bone discs were implanted subcutaneously into 10-week old female NOD SCID mice (n=10/group) under isofluorane anaesthetic. Mice received an injection of 0.003 mg vetergesic and Septrin was added to the drinking water for 1 week following bone implantation. Mice were left for 4 weeks before injecting 1×10⁵ MDA-MB-231 Luc2-TdTomato, MCF7 Luc2 or T47D Luc2 cells in 20% Martigel/79% PBS/1% toluene blue into the two hind mammary fat pads. Primary tumor growth and development of metastases was monitored weekly using an IVIS (Luminol) system (Caliper Life Sciences) following sub-cutaneous injection of 30 mg/ml D-luciferin (Invitrogen). On termination of experiments mammary tumors, circulating tumor cells, serum and bone metastases were resected. RNA was processed for downstream analysis by real time PCR, and cell lysates were taken for protein analysis and whole tissue for histology as previously described (Nutter et al., 2014; Ottewell et al., 2014a).
For therapeutic studies in NOD SCID mice, placebo (control), 1 mg/kg IL-1Ra (Anakinra®) daily or 10 mg/kg canakinumab subcutaneously every 14 days were administered starting 7 days after injection of tumor cells. In BALB/c mice and C57BL/6 mice 1 mg/kg IL-1Ra was administered daily for 21 or 31 days or 10 mg/kg canakinumab was administered as a single subcutaneous injection. Tumor cells, serum, and bone were subsequently resected for downstream analysis.
Bone metastases were investigated following injection of 5×10⁵ MDA-MB-231 GFP (control), MDA-MB-231-IV, MDA-MB-231-IL-1B-positive or MDA-MB-231-IL-1R1-positive cells into the lateral tail vein of 6 to 8-week old female BALB/c nude mice (n=12/group). Tumor growth in bones and lungs was monitored weekly by GFP imaging in live animals. Mice were culled 28 days after tumor cell injection at which timepoint hind limbs, lungs and serum were resected and processed for microcomputed tomography imaging (pCT), histology and ELISA analysis of bone turnover markers and circulating cytokines as described (Holen et al., 2016).

### Isolation of Circulating Tumor Cells

Whole blood was centrifuged at 10,000×g for 5 minutes and the serum removed for ELISA assays. The cell pellet was re-suspended in 5 ml of FSM lysis solution (Sigma-Aldrich, Pool, UK) to lyse red blood cells. Remaining cells were re-pelleted, washed 3× in PBS and re-suspended in a solution of PBS/10% FCS. Samples from 10 mice per group were pooled prior to isolation of TdTomato positive tumor cells using a MoFlow High performance cell sorter (Beckman Coulter, Cambridge UK) with the 470 nM laser line from a Coherent I-90C tenable argon ion (Coherent, Santa Clara, Calif.). TdTomato fluorescence was detected by a 555LP dichroic long pass and a 580/30 nm band pass filter. Acquisition and analysis of cells was performed using Summit 4.3 software. After sorting, cells were immediately placed in RNA protect cell reagent (Ambion, Paisley, Renfrew, UK) and stored at −80° C. before RNA extraction. For counting numbers of circulating tumor cells, TdTomato fluorescence was detected using a 561 nm laser and an YL1-A filter (585/16 emission filter). Acquisition and analysis of cells was performed using Attune N×T software.

### Microcomputed Tomography Imaging

Microcomputed tomography (pCT) analysis was carried out using a Skyscan 1172 x-ray-computed pCT scanner (Skyscan, Aartselar, Belgium) equipped with an x-ray tube (voltage, 49 kV; current, 200 uA) and a 0.5-mm aluminium filter. Pixel size was set to 5.86 μm and scanning initiated from the top of the proximal tibia as previously described (Ottewell et al., 2008a; Ottewell et al., 2008b).

### Bone Histology and Measurement of Tumor Volume

Bone tumor areas were measured on three non-serial, H&E stained, 5 μm histological sections of decalcified tibiae per mouse using a Leica RMRB upright microscope and Osteomeasure software (Osteometrics, Inc. Decauter, USA) and a computerised image analysis system as described previously (Ottewell et al., 2008a).

### Western Blotting

Protein was extracted using a mammalian cell lysis kit (Sigma-Aldrich, Poole, UK). 30 μg of protein was run on 4-15% precast polyacrylamide gels (BioRad, Watford, UK) and transferred onto an Immobilon nitrocellulose membrane (Millipore). Non-specific binding was blocked with 1% casein (Vector Laboratories) before incubation with rabbit monoclonal antibodies to human N-cadherin (D4R1H) at a dilution of 1:1000, E-cadherin (24E10) at a dilution of 1:500 or gamma-catenin (2303) at a dilution of 1:500 (Cell signalling) or mouse monoclonal GAPDH (ab8245) at a dilution of 1:1000 (AbCam, Cambridge UK) for 16 h at 4° C. Secondary antibodies were anti-rabbit or anti-mouse horse radish peroxidase (HRP; 1:15,000) and HRP was detected with the Supersignal chemiluminescence detection kit (Pierce). Band quantification was carried out using Quantity Once software (BioRad) and normalised to GAPDH.

### Gene Analysis

Total RNA was extracted using an RNeasy kit (Qiagen) and reverse transcribed into cDNA using Superscript III (Invitrogen AB). Relative mRNA expression of IL-1B (Hs02786624), IL-JR1 (Hs00174097), CASP (Caspase 1) (Hs00354836), IL1RN (Hs00893626), JUP (junction plakoglobin/gamma-catenin) (Hs00984034), N-cadherin (Hs01566408) and E-cadherin (Hs1013933) were compared with the housekeeping gene glyceraldehyde-3-phosphate dehydrogenase (GAPDH; Hs02786624) and assessed using an ABI 7900 PCR System (Perkin Elmer, Foster City, Calif.) and Taqman universal master mix (Thermofisher, UK). Fold change in gene expression between treatment groups was analysed by inserting CT values into Data Assist V3.01 software (Applied Biosystems) and changes in gene expression were only analysed for genes with a CT value of ≤25.
Assessment of IL-1β and IL-1R1 in Tumors from Breast Cancer Patients
IL-1β and IL-1R1 expression was assessed on tissue microarrays (TMA) containing primary breast tumor cores taken from 1,300 patients included in the clinical trial, AZURE (Coleman et al. 2011). Samples were taken pre-treatment from patients with stage II and III breast cancer without evidence of metastasis. Patients were subsequently randomized to standard adjuvant therapy with or without the addition of zoledronic acid for 10 years (Coleman et al 2011). The TMAs were stained for IL-1β (ab2105, 1:200 dilution, Abcam) and IL-1R1 (ab59995, 1:25 dilution, Abcam) and scored blindly under the guidance of a histopathologist for IL-1β/IL-1R1 in the tumor cells or in the associated stroma. Tumor or stromal IL-1β or IL-1R1 was then linked to disease recurrence (any site) or disease recurrence specifically in bone (+/− other sites).

### The IL-1β Pathway is Upregulated During the Process of Human Breast Cancer Metastasis to Human Bone.

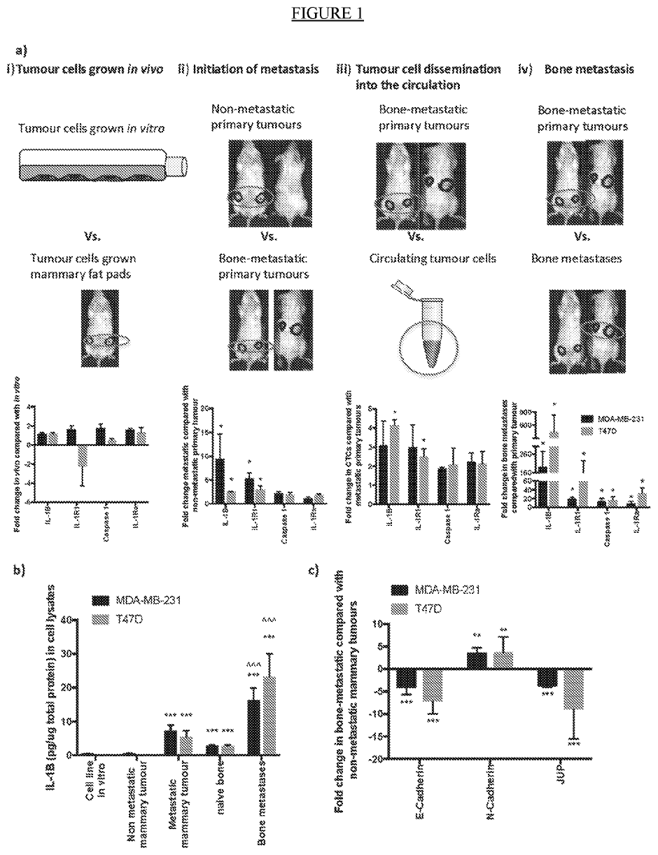

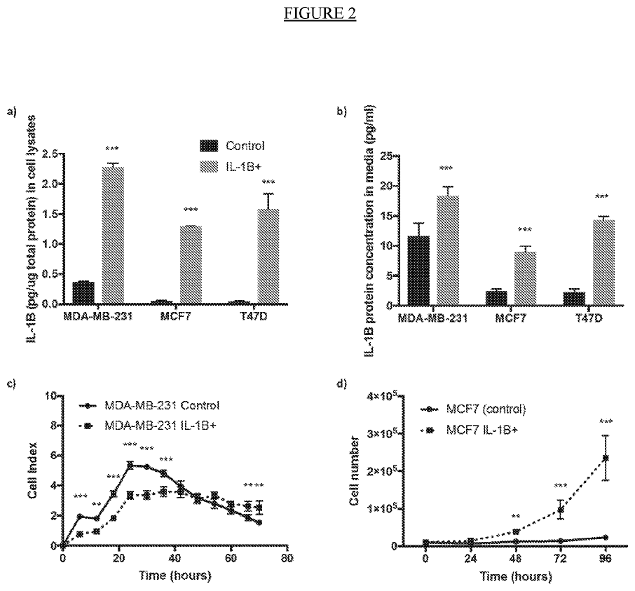

A mouse model of spontaneous human breast cancer metastasis to human bone implants was utilised to investigate how the IL-1β pathway changes through the different stages of metastasis. Using this model, the expression levels of genes associated with the IL-1β pathway increased in a stepwise manner at each stage of the metastatic process in both triple negative (MDA-MB-231) and estrogen receptor positive (ER+ve) (T47D) breast cancer cells: Genes associated with the IL-1β signalling pathway (IL-1B, IL-1R1, CASP (Caspase 1) and IL-1Ra) were expressed at very low levels in both MDA-MB-231 and T47D cells grown in vitro and expression of these genes were not altered in primary mammary tumors from the same cells that did not metastasize in vivo (FIG. 1a).
IL-1B, IL-1R1 and CASP were all significantly increased in mammary tumors that subsequently metastasized to human bone compared with those that did not metastasize (p<0.01 for both cell lines), leading to activation of IL-1β signalling as shown by ELISA for the active 17 kD IL-1β (FIG. 1b; FIG. 2). IL-1B gene expression increased in circulating tumor cells compared with metastatic mammary tumors (p<0.01 for both cell lines) and IL-1B (p<0.001), IL-1R1 (p<0.01), CASP (p<0.001) and IL-1Ra (p<0.01) were further increased in tumor cells isolated from metastases in human bone compared with their corresponding mammary tumors, leading to further activation of IL-1β protein (FIG. 1; FIG. 2). These data suggest that IL-1β signalling may promote both initiation of metastasis from the primary site as well as development of breast cancer metastases in bone.

### Tumor Derived IL-1β Promotes EMT and Breast Cancer Metastasis.

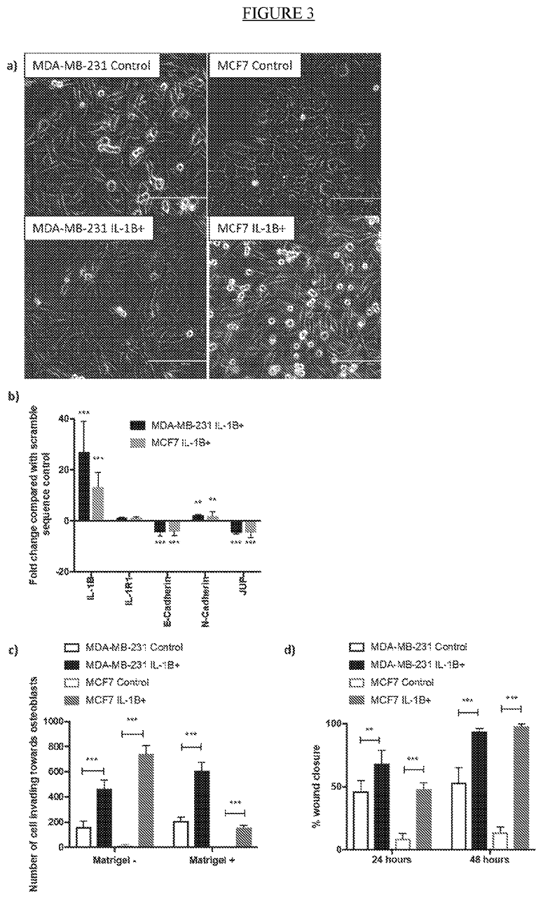

Expression levels of genes associated with tumor cell adhesion and epithelial to mesenchymal transition (EMT) were significantly altered in primary tumors that metastasized to bone compared with tumors that did not metastasise (FIG. 1c). IL-1β-overexpressing cells were generated (MDA-MB-231-IL-1B+, T47D-IL-1B+ and MCF7-IL-1B+) to investigate whether tumor-derived IL-1β is responsible for inducing EMT and metastasis to bone. All IL-1β+ cell lines demonstrated increased EMT exhibiting morphological changes from an epithelial to mesenchymal phenotype (FIG. 3a) as well as reduced expression of E-cadherin, and JUP (junction plakoglobin/gamma-catenin) and increased expression of N-Cadherin gene and protein (FIG. 3b). Wound closure (p<0.0001 in MDA-MB-231-IL-1β+(FIG. 3d); p<0.001 MCF7-IL-1β+ and T47D-IL-1β+) and migration and invasion through matrigel towards osteoblasts were increased in tumor cells with increased IL-1β signalling compared with their respective controls (MDA-MB-231-IL-1β+(FIG. 3c) p<0.0001; MCF7-IL-1β+ and T47D-IL-1β+p<0.001). Increased IL-1β production was seen in ER-positive and ER-negative breast cancer cells that spontaneously metastasized to human bone implants in vivo compared with non-metastatic breast cancer cells (FIG. 1). The same link between IL-1β and metastasis was made in primary tumor samples from patients with stage II and III breast cancer enrolled in the AZURE study (Coleman et al., 2011) that experienced cancer relapsed over a 10 year time period. IL-1β expression in primary tumors from the AZURE patients correlated with both relapse in bone and relapse at any site indicating that presence of this cytokine is likely to play a role in metastasis in general. In agreement with this, genetic manipulation of breast cancer cells to artificially overexpress IL-1β increased the migration and invasion capacities of breast cancer cells in vitro (FIG. 3).

### Inhibition of IL-1β Signaling Reduces Spontaneous Metastasis to Human Bone.

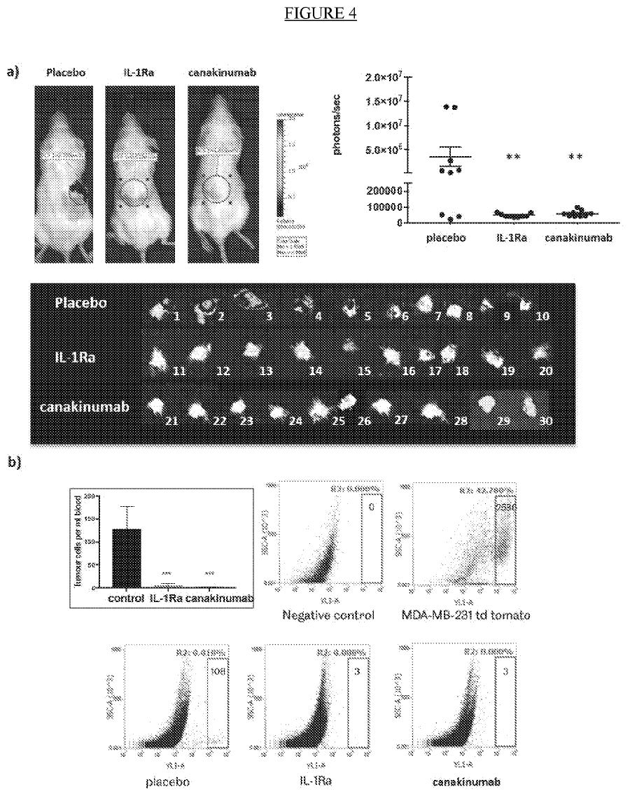

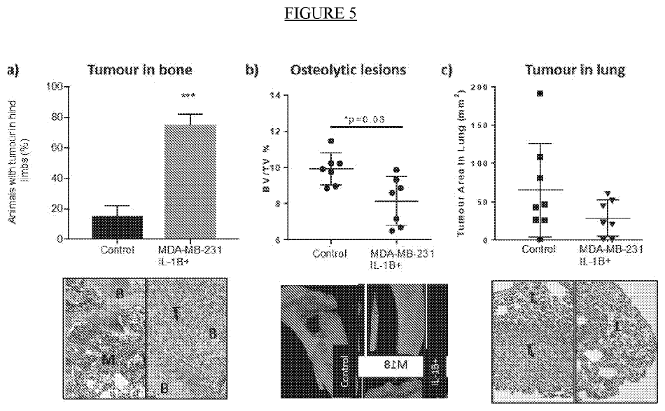

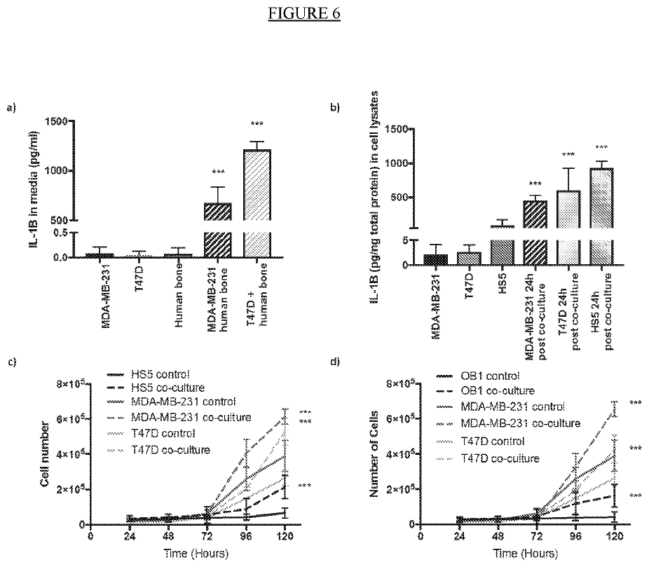

As tumor derived IL-1β appeared to be promoting onset of metastasis through induction of EMT the effects of inhibiting IL-1β signaling with IL-1Ra (Anakinra) or a human anti-IL-1β-binding antibody (canakinumab) on spontaneous metastasis to human bone implants were investigated: Both IL-1Ra and canakinumab reduced metastasis to human bone: metastasis was detected in human bone implants in 7 out of 10 control mice, but only in 4 out of 10 mice treated with IL-1Ra and 1 out of 10 mice treated with canakinumab. Bone metastases from IL-1Ra and canakinumab treatment groups were also smaller than those detected in the control group (FIG. 4a). Numbers of cells detected in the circulation of mice treated with canakinumab or IL-1Ra were significantly lower than those detected in the placebo treated group: only 3 tumor cells/ml were counted in whole blood from mice treated with canakinumab and anakinra, respectively, compared 108 tumor cells/ml counted in blood from placebo treated mice (FIG. 4b), suggesting that inhibition of IL-1 signalling prevents tumor cells from being shed from the primary site into the circulation. Therefore, inhibition of IL-1β signaling with the anti-IL-1β antibody canakinumab or inhibition of IL-1R1 reduced the number of breast cancer cells shed into the circulation and reduced metastases in human bone implants (FIG. 4).

### Tumor Derived IL-1β Promotes Bone Homing and Colonisation of Breast Cancer Cells.

Injection of breast cancer cells into the tail vein of mice usually results in lung metastasis due to the tumor cells becoming trapped in the lung capillaries. It was previously shown that breast cancer cells that preferentially home to the bone microenvironment following intra-venous injection express high levels of IL-1β, suggesting that this cytokine may be involved in tissue specific homing of breast cancer cells to bone. In the current study, intravenous injection of MDA-MB-231-IL-1β+ cells into BALB/c nude mice resulted in significantly increased number of animals developing bone metastasis (75%) compared with control cells (12%) (p<0.001) cells (FIG. 5a). MDA-MB-231-IL-1β+ tumors caused development of significantly larger osteolytic lesions in mouse bone compared with control cells (p=0.03; FIG. 5b) and there was a trend towards fewer lung metastases in mice injected with MDA-MB-231-IL-1β+ cells compared with control cells (p=0.16; FIG. 5c). These data suggest that endogenous IL-1β can promote tumor cell homing to the bone environment and development of metastases at this site.

### Tumor Cell-Bone Cell Interactions Further Induce IL-1B and Promote Development of Overt Metastases.

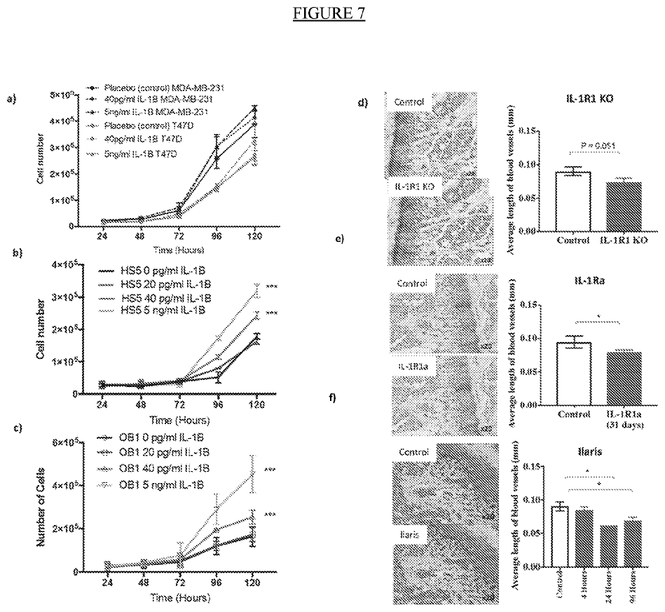

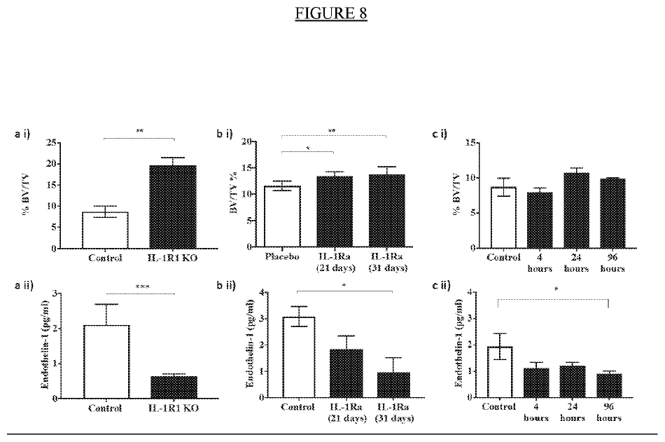

Gene analysis data from a mouse model of human breast cancer metastasis to human bone implants suggested that the IL-1β pathway was further increased when breast cancer cells are growing in the bone environment compared with metastatic cells in the primary site or in the circulation (FIG. 1a). It was therefore investigated how IL-1β production changes when tumor cells come into contact with bone cells and how IL-1β alters the bone microenvironment to affect tumor growth (FIG. 6). Culture of human breast cancer cells into pieces of whole human bone for 48 h resulted in increased secretion of IL-1β into the medium (p<0.0001 for MDA-MB-231 and T47D cells; FIG. 6a). Co-culture with human HS5 bone marrow cells revealed the increased IL-1β concentrations originated from both the cancer cells (p<0.001) and bone marrow cells (p<0.001), with IL-1β from tumor cells increasing ˜1000 fold and IL-1β from HS5 cells increasing ˜100 fold following co-culture (FIG. 6b).
Exogenous IL-1β did not increase tumor cell proliferation, even in cells overexpressing IL-1R1. Instead, IL-1β stimulated proliferation of bone marrow cells, osteoblasts and blood vessels that in turn induced proliferation of tumor cells (FIG. 6). It is therefore likely that arrival of tumor cells expressing high concentrations of IL-1β stimulate expansion of the metastatic niche components and contact between IL-1β expressing tumor cells and osteoblasts/blood vessels drive tumor colonization of bone. The effects of exogenous IL-1β as well as IL-1β from tumor cells on proliferation of tumor cells, osteoblasts, bone marrow cells and CD34+ blood vessels were investigated: Co-culture of HS5 bone marrow or OB1 primary osteoblast cells with breast cancer cells caused increased proliferation of all cell types (P<0.001 for HS5, MDA-MB-231 or T47D, FIG. 6c) (P<0.001 for OB1, MDA-MB-231 or T47D, FIG. 6d). Direct contact between tumor cells, primary human bone samples, bone marrow cells or osteoblasts promoted release of IL-1β from both tumor and bone cells (FIG. 6). Furthermore, administration of IL-1β increased proliferation of HS5 or OB1 cells but not breast cancer cells (FIG. 7 a-c), suggesting that tumor cell-bone cell interactions promote production of IL-1β that can drive expansion of the niche and stimulate the formation of overt metastases.
IL-1β signalling was also found to have profound effects on the bone microvasculature: Preventing IL-1β signaling in bone by knocking out IL-1R1, pharmacological blockade of IL-1R with IL-1Ra or reducing circulating concentrations of IL-1β by administering the anti-IL-1β binding antibody canakinumab reduced the average length of CD34⁺ blood vessels in trabecular bone, where tumor colonisation takes place (p<0.01 for IL-1Ra and canakinumab treated mice) (FIG. 7c). These findings were confirmed by endomucin staining which showed decreased numbers of blood vessels as well as blood vessel length in bone when IL-1β signaling was disrupted. ELISA analysis for endothelin 1 and VEGF showed reduced concentrations of both of these endothelial cell markers in the bone marrow for IL-1R1^{-/-} mice (p<0.001 endothelin 1; p<0.001 VEGF) and mice treated with IL-1R antagonist (p<0.01 endothlin 1; p<0.01 VEGF) or canakinumab (p<0.01 endothelin 1; p<0.001 VEGF) compared with control (FIG. 8). These data suggest that tumor cell-bone cell associated increases in IL-1β and high levels of IL-1β in tumor cells may also promote angiogenesis, further stimulating metastases.

### Tumor Derived IL-1β Predicts Future Breast Cancer Relapse in Bone and Other Organs in Patient Material

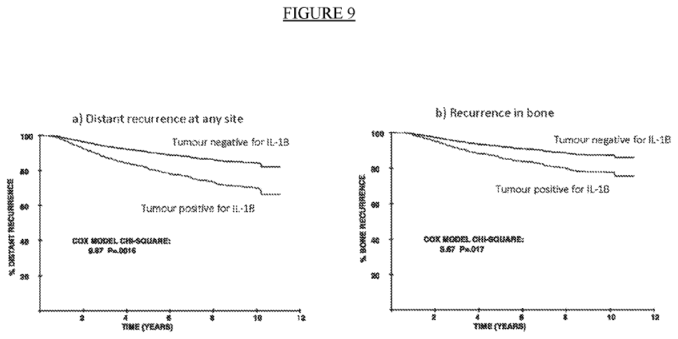

To establish the relevance of the findings in a clinical setting the correlation between IL-1β and its receptor IL-1R1 in patient samples was investigated. ˜1300 primary tumor samples from patients with stage II/III breast cancer with no evidence of metastasis (from the AZURE study (Coleman et al., 2011)) were stained for IL-1R1 or the active (17 kD) form of IL-1β, and biopsies were scored separately for expression of these molecules in the tumor cells and the tumor associated stroma. Patients were followed up for 10 years following biopsy and correlation between IL-1β/IL-1R1 expression and distant recurrence or relapse in bone assessed using a multivariate Cox model. IL-1β in tumor cells strongly correlated with distant recurrence at any site (p=0.0016), recurrence only in bone (p=0.017) or recurrence in bone at any time (p=0.0387) (FIG. 9). Patients who had IL-1β in their tumor cells and IL-1R1 in the tumor associated stroma were more likely to experience future relapse at a distant site (p=0.042) compared to patients who did not have IL-1β in their tumor cells, indicating that tumor derived IL-1β may not only promote metastasis directly but may also interact with IL-1R1 in the stroma to promote this process. Therefore, IL-1β is a novel biomarker that can be used to predict risk of breast cancer relapse.

### Example 2

Simulation of Canakinumab PK Profile and hsCRP Profile for Lung Cancer Patients.
A model was generated to characterize the relationship between canakinumab pharmacokinetics (PK) and hsCRP based on data from the CANTOS study.
The following methods were used in this study: Model building was performed using the first-order conditional estimation with interaction method. The model described the logarithm of the time resolved hsCRP as:

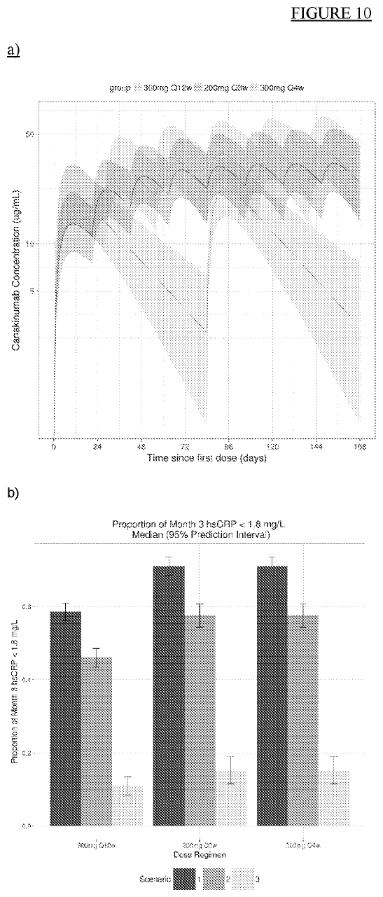

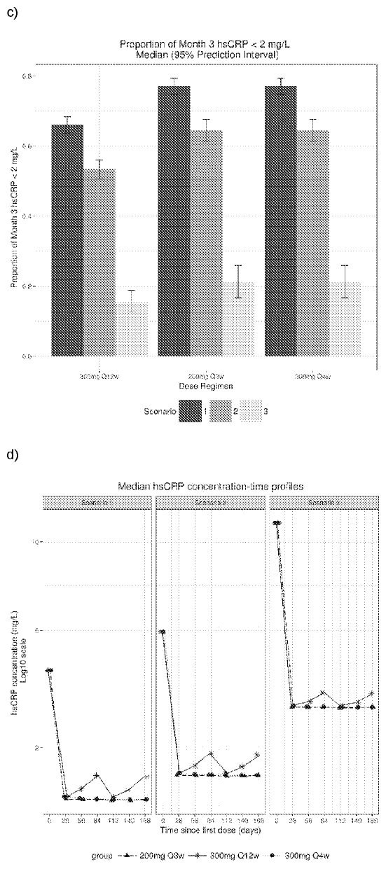

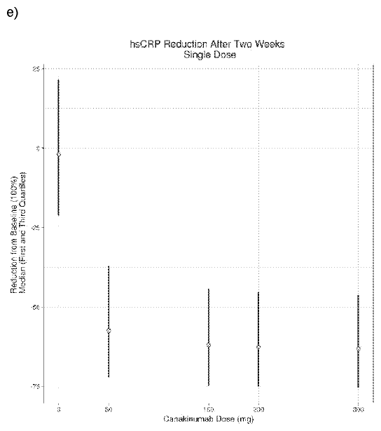

y(tᵢⱼ)=y_{0,i}+y_{eff}(tᵢⱼ)

where y_{0,i} is a steady state value and y_{eff}(tᵢⱼ) describes the effect of the treatment and depends on the systemic exposure. The treatment effect was described by an Emax-type model,

$${y}_{eff} ( {t}_{i j} ) = {E}_{max , i} \frac{c ( {t}_{i j} )}{c ( {t}_{i j} ) + IC {50}_{i}}$$

where E_{max,i} is the maximal possible response at high exposure, and IC50ᵢ is the concentration at which half maximal response is obtained.
The individual parameters, E_{max,i} and y_{0,i} and the logarithm of IC50ᵢ were estimated as a sum of a typical value, covariate effects covpar*covᵢ and normally distributed between subject variability. In the term for the covariate effect covpar refers to the covariate effect parameter being estimated and covᵢ is the value of the covariate of subject i. Covariates to be included were selected based on inspection of the eta plots versus covariates. The residual error was described as a combination of proportional and additive term.
The logarithm of baseline hsCRP was included as covariate on all three parameters (E_{max,i}, y_{0,i} and IC50ᵢ). No other covariate was included into the model. All parameters were estimated with good precision. The effect of the logarithm of the baseline hsCRP on the steady state value was less than 1 (equal to 0.67). This indicates that the baseline hsCRP is an imperfect measure for the steady state value, and that the steady state value exposes regression to the mean relative to the baseline value. The effects of the logarithm of the baseline hsCRP on IC50 and Emax were both negative. Thus patients with high hsCRP at baseline are expected to have low IC50 and large maximal reductions. In general, model diagnostics confirmed that the model describes the available hsCRP data well.
The model was then used to simulate expected hsCRP response for a selection of different dosing regimens in a lung cancer patient population. Bootstrapping was applied to construct populations with intended inclusion/exclusion criteria that represent potential lung cancer patient populations. Three different lung cancer patient populations described by baseline hsCRP distribution alone were investigated: all CANTOS patients (scenario 1), confirmed lung cancer patients (scenario 2), and advanced lung cancer patients (scenario 3).
The population parameters and inter-patient variability of the model were assumed to be the same for all three scenarios. The PK/PD relationship on hsCRP observed in the overall CANTOS population was assumed to be representative for lung cancer patients.
The estimator of interest was the probability of hsCRP at end of month 3 being below a cut point, which could be either 2 mg/L or 1.8 mg/L. 1.8 mg/L was the median of hsCRP level at end of month 3 in the CANTOS study. Baseline hsCRP>2 mg/L was one of the inclusion criteria, so it is worthy to explore if hsCRP level at end of month 3 went below 2 mg/L. A one-compartment model with first order absorption and elimination was established for CANTOS PK data. The model was expressed as ordinary differential equation and R×ODE was used to simulate canakinumab concentration time course given individual PK parameters. The subcutaneous canakinumab dose regimens of interest were 300 mg Q12W, 200 mg Q3W, and 300 mg Q4W. Exposure metrics including Cmin, Cmax, AUCs over different selected time periods, and average concentration Cave at steady state were derived from simulated concentration time profiles.
The simulation in Scenario 1 was based on the below information:
Individual canakinumab exposure simulated using R×ODE
PD parameters which are components of y_{0,i}, E_{max,i}, and IC50ᵢ: typical values (THETA(3), THETA(5), THETA(6)), covpars (THETA(4), THETA(7), THETA(8), and between subject variability (ETA(1), ETA(2), ETA(3))
Baseline hsCRP from all 10,059 CANTOS study patients (baseline hsCRP: mean 6.18 mg/L, standard error of the mean (SEM)=0.10 mg/L)
The prediction interval of the estimator of interest was produced by first randomly sampling 1000 THETA(3)-(8)s from a normal distribution with fixed mean and standard deviation estimated from the population PK/PD model; and then for each set of THETA(3)-(8), bootstrapping 2000 PK exposure, PD parameters ETA(1)-(3), and baseline hsCRP from all CANTOS patients. The 2.5%, 50%, and 97.5% percentile of 1000 estimates were reported as point estimator as well as 95% prediction interval.
The simulation in Scenario 2 was based on the below information:
Individual canakinumab PK exposure simulated using R×ODE
PD parameters THETA(3)-(8) and ETA(1)-(3)
Baseline hsCRP from 116 CANTOS patients with confirmed lung cancer (baseline hsCRP: mean=9.75 mg/L, SEM=1.14 mg/L)
The prediction interval of the estimator of interest was produced by first randomly sampling 1000 THETA(3)-(8)s from a normal distribution with fixed mean and standard deviation estimated from the population PKPD model; and then for each set of THETA(3)-(8), bootstrapping 2000 PK exposure, PD parameters ETA(1)-(3) from all CANTOS patients, and bootstrapping 2000 baseline hsCRP from the 116 CANTOS patients with confirmed lung cancer. The 2.5%, 50%, and 97.5% percentile of 1000 estimates were reported as point estimator as well as 95% prediction interval.
In Scenario 3, the point estimator and 95% prediction interval were obtained in a similar manner as for scenario 2. The only difference was bootstrapping 2000 baseline hsCRP values from advanced lung cancer population. There is no individual baseline hsCRP data published in an advanced lung cancer population. An available population level estimate in advanced lung cancer is a mean of baseline hsCRP of 23.94 mg/L with SEM 1.93 mg/L [Vaguliene 2011]. Using this estimate, the advanced lung cancer population was derived from the 116 CANTOS patients with confirmed lung cancer using an additive constant to adjust the mean value to 23.94 mg/L.
In line with the model, the simulated canakinumab PK was linear. The median and 95% prediction interval of concentration time profiles are plotted in natural logarithm scale over 6 months is shown in FIG. 10a.
The median and 95% prediction intervals of 1000 estimates of proportion of subjects with month 3 hsCRP response under the cut point of 1.8 mg/L and 2 mg/L mhsCRP are reported in FIGS. 10b and c. Judging from the simulation data, 200 mg Q3W and 300 mg Q4W perform similarly and better than 300 mg Q12W (top dosing regimen in CANTOS) in terms of decreasing hsCRP at month 3. Going from scenario 1 to scenario 3 towards more severe lung cancer patients, higher baseline hsCRP levels are assumed, and result in smaller probabilities of month 3 hsCRP being below the cut point. FIG. 10d shows how the median hsCRP concentration changes over time for three different doses and FIG. 10e shows the percent reduction from baseline hsCRP after a single dose.

### Example 3A

### PDR001 Plus Canakinumab Treatment Increases Effector Neutrophils in Colorectal Tumors.

RNA sequencing was used to gain insights on the mechanism of action of canakinumab (ACZ885) in cancer. The CPDR001X2102 and CPDR001X2103 clinical trials evaluate the safety, tolerability and pharmacodynamics of spartalizumab (PDR001) in combination with additional therapies. For each patient, a tumor biopsy was obtained prior to treatment, as well as cycle 3 of treatment. In brief, samples were processed by RNA extraction, ribosomal RNA depletion, library construction and sequencing. Sequence reads were aligned by STAR to the hg19 reference genome and Refseq reference transcriptome, gene-level counts were compiled by HTSeq, and sample-level normalization using the trimmed mean of M-values was performed by edgeR.

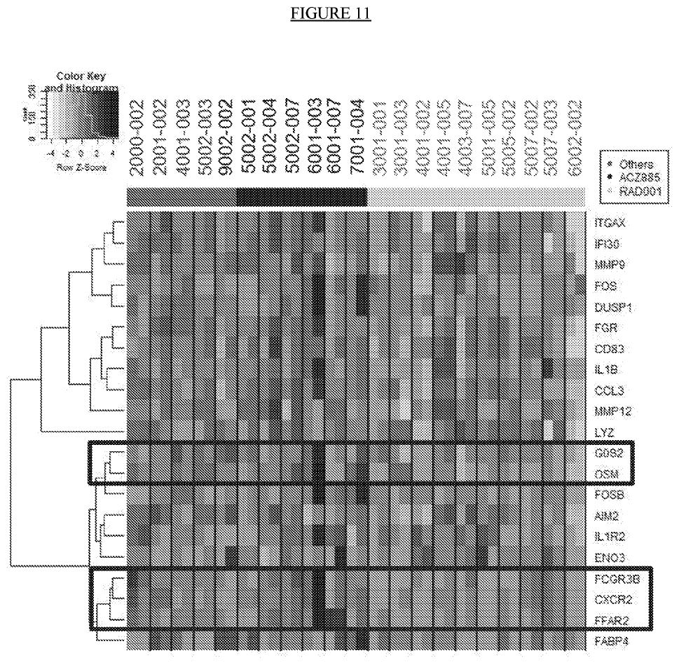

FIG. 11 shows 21 genes that were increased, on average, in colorectal tumors treated with PDR001+canakinumab (ACZ885), but not in colorectal tumors treated with PDR001+everolimus (RAD001). Treatment with PDR001+canakinumab increased the RNA levels of IL1B, as well as its receptor, IL1R2. This observation suggests an on-target compensatory feedback by tumors to increase IL1B RNA levels in response to IL-1β protein blockade.

Notably, several neutrophil-specific genes were increased on PDR001+canakinumab, including FCGR3B, CXCR2, FFAR2, OSM, and G0S2 (indicated by boxes in FIG. 11). The FCGR3B gene is a neutrophil-specific isoform of the CD16 protein. The protein encoded by FCGR3B plays a pivotal role in the secretion of reactive oxygen species in response to immune complexes, consistent with a function of effector neutrophils (Fossati G 2002 Arthritis Rheum 46: 1351). Chemokines that bind to CXCR2 mobilize neutrophils out of the bone marrow and into peripheral sites. In addition, increased CCL3 RNA was observed on treatment with PDR001+canakinumab. CCL3 is a chemoattractant for neutrophils (Reichel C A 2012 Blood 120: 880).

In summary, this contribution of components analysis using RNA-seq data demonstrates that PDR001+canakinumab treatment increases effector neutrophils in colorectal tumors, and that this increase was not observed with PDR001+everolimus treatment.

### Example 3B

Efficacy of Canakinumab (ACZ885) in Combination with Spartalizumab (PDR001) in the Treatment of Cancer.

Patient 5002-004 is a 56 year old man with initially Stage IIC, microsatellite-stable, moderately differentiated adenocarcinoma of the ascending colon (MSS-CRC), diagnosed in June, 2012 and treated with prior regimens.

Prior treatment regimens included:
1. Folinic acid/5-fluoruracil/oxaliplatin in the adjuvant setting
2. Chemoradiation with capecitabine (metastatic setting)
3. 5-fluorouracil/bevacizumab/folinic acid/irinotecan
4. trifluridine and tipiracil
5. Irinotecan
6. Oxaliplatin/5-fluorouracil
7. 5-fluorouracil/bevacizumab/leucovorin
8. 5-fluorouracil

At study entry the patient had extensive metastatic disease including multiple hepatic and bilateral lung metastases, and disease in paraesophageal lymph nodes, retroperitoneum and peritoneum.

The patient was treated with PDR001 400 mg every four weeks (Q4W) plus 100 mg every eight weeks (Q8W) ACZ885. The patient had stable disease for 6 months of therapy, then with substantial disease reduction and confirmed RECIST partial response to treatment at 10 months. The patient has subsequently developed progressive disease and the dose was increased to 300 mg and then to 600 mg.

### Example 4

### Calculations for Selecting the Dose for Gevokizumab for Cancer Patients.

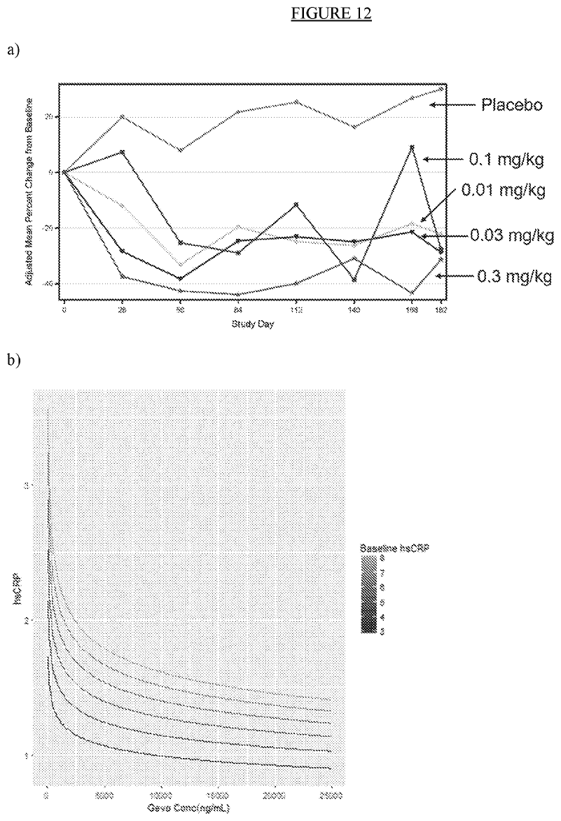

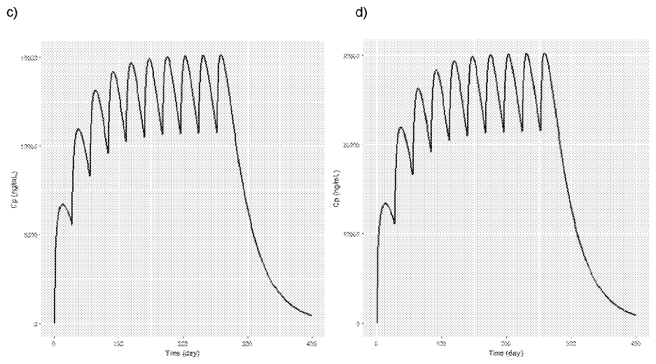

Dose selection for gevokizumab in the treatment of cancer having at least partial inflammatory basis is based on the clinical effective dosings reveals by the CANTOS trial in combination with the available PK data of gevokizumab, taking into the consideration that Gevokizumab (IC50 of ˜2-5 μM) shows a ˜10 times higher in vitro potency compared to canakinumab (IC50 of ˜42±3.4 μM). The gevokizumab top dose of 0.3 mg/kg (˜20 mg) Q4W showed reduction of hsCRP could reduce hsCRP up to 45% in type 2 diabetes patients (see FIG. 12a).

Next, a pharmacometric model was used to explore the hsCRP exposure-response relationship, and to extrapolate the clinical data to higher ranges. As clinical data show a linear correlation between the hsCRP concentration and the concentration of gevokizumab (both in log-space), a linear model was used. The results are shown in FIG. 12b. Based on that simulation, a gevokizumab concentration between 10000 ng/mL and 25000 ng/mL is optimal because hsCRP is greatly reduced in this range, and there is only a diminishing return with gevokizumab concentrations above 15000 ng/mL. However gevokizumab concentrations between 4000 ng/mL and 10000 ng/mL is expected to be efficacious as hsCRP has already been significantly reduced in that range.
Clinical data showed that gevokizumab pharmacokinetics follow a linear two-compartment model with first order absorption after a subcutaneous administration. Bioavailability of gevokizumab is about 56% when administered subcutaneously. Simulation of multiple-dose gevokizumab (SC) was carried out for 100 mg every four weeks (see FIG. 12c) and 200 mg every four weeks (see FIG. 12d). The simulations showed that the trough concentration of 100 mg gevokizumab given every four weeks is about 10700 ng/mL. The half-life of gevokizumab is about 35 days. The trough concentration of 200 mg gevokizumab given every four weeks is about 21500 ng/mL.

### Example 5

### Preclinical Data on the Effects of Anti-IL-1β Treatment.

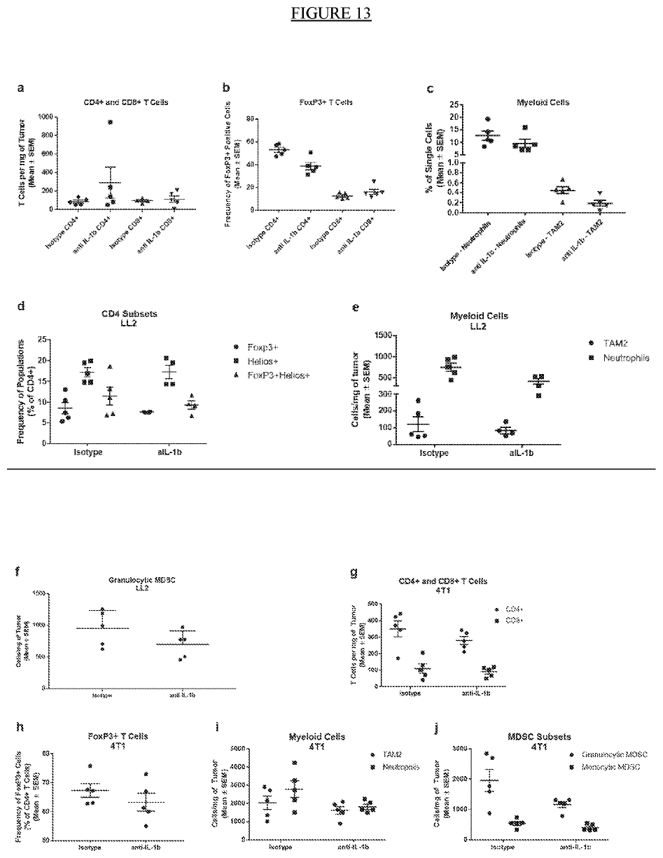

Canakinumab, an anti-IL-1β human IgG1 antibody, cannot directly be evaluated in mouse models of cancer due to the fact that it does not cross-react with mouse IL-1β. A mouse surrogate anti-IL-1β antibody has been developed and is being used to evaluate the effects of blocking IL-1β in mouse models of cancer. This isotype of the surrogate antibody is IgG2a, which is closely related to human IgG1.
In the MC38 mouse model of colon cancer, modulation of tumor infiltrating lymphocytes (TILs) can be seen after one dose of the anti IL-1β antibody (FIG. 13a-c). MC38 tumors were subcutaneously implanted in the flank of C57BL/6 mice and when the tumors were between 100-150 mm3, the mice were treated with one dose of either an isotype antibody or the anti IL-1β antibody. Tumors were then harvested five days after the dose and processed to obtain a single cell suspension of immune cells. The cells were then ex vivo stained and analyzed via flow cytometry. Following a single dose of an IL-1β blocking antibody, there is an increase in in CD4+ T cells infiltrating the tumor and also a slight increase in CD8+ T cells (FIG. 13a). The CD8+ T cell increase is slight but may allude to a more active immune response in the tumor microenvironment, which could potentially be enhanced with combination therapies. The CD4+ T cells were further subdivided into FoxP3+regulatory T cells (Tregs), and this subset decreases following blockade of IL-1β (FIG. 13b). Among the myeloid cell populations, blockade of IL-1β results in a decrease in neutrophils and the M2 subset of macrophages, TAM2 (FIG. 13c). Both neutrophils and M2 macrophages can be suppressive to other immune cells, such as activated T cells (Pillay et al, 2013; Hao et al, 2013; Oishi et al 2016). Taken together, the decrease in Tregs, neutrophils, and M2 macrophages, in the MC38 tumor microenvironment following IL-1β blockade argues that the tumor microenvironment is becoming less immune suppressive.
In the LL2 mouse model of lung cancer, a similar trend towards a less suppressive immune microenvironment can be seen after one dose of an anti-IL-1β antibody (FIG. 13d-f). LL2 tumors were subcutaneously implanted in the flank of C57BL/6 mice and when the tumors were between 100-150 mm3, the mice were treated with one dose of either an isotype antibody or the anti-IL-1β antibody. Tumors were then harvested five days after the dose and processed to obtain a single cell suspension of immune cells. The cells were then ex vivo stained and analyzed via flow cytometry. There is a decrease in the Treg populations as evaluated by the expression of FoxP3 and Helios (FIG. 13d). FoxP3 and Helios are both used as markers of regulatory T cells, while they may define different subsets of Tregs (Thorton et al, 2016). Similar to the MC38 model, there is a decrease in both neutrophils and M2 macrophages (TAM2) following IL-1β blockade (FIG. 13e). In addition to this, in this model the change in the myeloid derived suppressor cell (MDSC) populations were evaluated following antibody treatment. The granulocytic or polymorphonuclear (PMN) MDSC were found in reduced numbers following anti-IL-1β treatment (FIG. 13f). MDSC are a mixed population of cells of myeloid origin that can actively suppress T cell responses through several mechanisms, including arginase production, reactive oxygen species (ROS) and nitric oxide (NO) release (Kumar et al, 2016; Umansky et al, 2016). Again, the decrease in Tregs, neutrophils, M2 macrophages, and PMN MDSC in the LL2 model following IL-1β blockade argues that the tumor microenvironment is becoming less immune suppressive.
TILs in the 4T1 triple negative breast cancer model also show a trend towards a less suppressive immune microenvironment after one dose of the mouse surrogate anti-IL-1β antibody (FIG. 13g-j). 4T1 tumors were subcutaneously implanted in the flank of Balb/c mice, and the mice were treated with either an isotype antibody or the anti-IL-1β antibody when the tumors were between 100-150 mm3. Tumors were then harvested five days after the dose and processed to obtain a single cell suspension of immune cells. The cells were then ex vivo stained and analyzed via flow cytometry. There is a decrease in CD4+ T cells after a single dose of an anti-IL-1β antibody (FIG. 13g) and within the CD4+ T cell population, there is a decrease in the FoxP3+ Tregs (FIG. 13h). Further, there is a decrease in both the TAM2 and neutrophil populations following treatment of the tumor-bearing mice (FIG. 13i). All of these data together again argue that IL-1β blockade in the 4T1 breast cancer mouse model leads to a less suppressive immune microenvironment. In addition to this, in this model the MDSC populations was also evaluated following antibody treatment. Both the granulocytic (PMN) MDSC and monocytic MDSC were found in reduced numbers following anti-IL-1β treatment (FIG. 13j). These findings in combination with the changes in Tregs, M2 macrophages, and the neutrophil populations describe a decrease in the immune suppressive tumor microenvironment in the 4T1 tumor model.
While these data are from colon, lung, and breast cancer models, the data can be extrapolated to other types of cancer. Even though these models do not fully correlate to human cancers of the same type, the MC38 model in particular is a good surrogate model for hypermutated/MSI (microsatellite instable) colorectal cancer (CRC). Based on the transcriptomic characterization of the MC38 cell line, four of the driver mutations in this line correspond to known hotspots in human CRC, although these are at different positions (Efremova et al, 2018). While this does not make the MC38 mouse model identical to human CRC, it does mean that MC38 may be a relevant model for human MSI CRC. Generally, mouse models do not always correlate to the same type of cancer in humans due to genetic differences in the origins of the cancer in mice versus humans. However, when examining the infiltrating immune cells, the type of cancer is not always important, as the immune cells are more relevant. In this case, as three different mouse models show a similar decrease in the suppressive microenvironment of the tumor, blocking IL-1β seems to lead to a less suppressive tumor microenvironment. The extent of the change in immune suppression with multiple cell types (Tregs, TAMs, neutrophils) showing a decrease compared to the isotype control in multiple tumor syngeneic mouse tumor models is a novel finding for IL-1β blockade in mouse models of cancer. While suppressor cell decreases have been seen before, multiple cell types in each model is a novel finding. In addition, changes to MDSC populations in the 4T1 and Lewis lung carcinoma (LL2) models have been seen downstream of IL-1β, but the finding in the LL2 model that blockade of IL-1β can lead to the reduction of MDSCs is novel to this study and the mouse surrogate of canakinumab (Elkabets et al, 2010).
Even though these models do not fully correlate to human cancers of the same type, the MC38 model in particular is a good surrogate model for hypermutated/MSI (microsatellite instable) colorectal cancer (CRC). Based on the transcriptomic characterization of the MC38 cell line, four of the driver mutations in this line correspond to known hotspots in human CRC, although these are at different positions (Efremova et al, 2018). While this does not make the MC38 mouse model identical to human CRC, it does mean that MC38 may be a relevant model for human MSI CRC (Efremova M, et al. Nature Communications 2018; 9: 32)

### Example 6

A Phase 1b Study of Gevokizumab in Combination with Standard of Care Therapy in Patients with First and Second Line Metastatic Colorectal Cancer (mCRC), Second Line Metastatic Gastroesophageal Carcinoma, and Second or Third Line Metastatic Renal Cell Carcinoma (mRCC)
The study population includes patients in four cohorts:
Cohort A: first line mCRC: Patients with metastatic colorectal adenocarcinoma who have had no prior systemic treatment for metastatic intent.
Cohort B: second line mCRC: Patients have progressed on one prior line of chemotherapy in the metastatic disease setting. The prior line chemotherapy must include at least a fluoropyrimidine and oxaliplatin. Maintenance therapy are not counted as a separate line of therapy. Patients have had no prior exposure to irinotecan. Patients have no history of Gilbert's Syndrome, or any of the following genotypes: UGT1A1*6/*6, UGT1A1*28/*28, or UGT1A1*6/*28.
Cohort C: second line metastatic gastroesophageal cancer: Patients have locally advanced, unresectable or metastatic gastric or gastroesophageal junction adenocarcinoma (not squamous cell), which has progressed on first-line systemic therapy with any platinum/fluoropyrimidine doublet, with or without anthracycline (epirubicin or doxorubicin). The patient has not received any previous systemic therapy targeting VEGF or the VEGFR signaling pathways. Serum hs-CRP level must be ≥10 mg/L for inclusion in the expansion cohort.
Cohort D: second or third line mRCC: Patients have mRCC with a clear-cell component and have received one or two lines of systemic treatment for mRCC. At least one line of treatment has to include anti-angiogenic therapy for at least 4 weeks (single agent or in combination) and with radiographic progression during this line of treatment. Patients have not received prior cabozantinib. Serum hs-CRP level must be ≥10 mg/L for inclusion in the expansion cohort.

### Dose Finding Part (Part 1a)

Dose finding part (gevokizumab dose 30 mg, 60 mg or 120 mg) will start the study and will recruit subjects in Cohorts A and B only with elevated baseline hs-CRP (hs-CRP≥10 mg/L). The purpose of this part is to determine the pharmacodynamically-active dose (PAD) of gevokizumab, which is the lowest dose of gevokizumab as monotherapy that results in close to maximal hs-CRP reduction at Day 15 of a 28-day cycle. After the end of Part 1a, subjects will enter seamlessly into Part 1b (see below) and will continue to receive gevokizumab (at the same dose as in Part 1a) in combination with the standard of care (SOC) anti-cancer therapies. A Bayesian approach will be utilized to guide the decision and determine the gevokizumab PAD. This approach will model the log(post baseline/baseline) values in hs-CRP, i.e. the hs-CRP change from baseline in log scale, where log is the natural logarithm.

### Safety Run-In Part (Part 1b)

Part 1b will include four cohorts (A, B, C, D) of subjects. The purpose of Part 1b is to determine, per cohort, the gevokizumab recommended dose for expansion (RDE), when given in combination with the SOC anti-cancer therapies.
The SOC anti-cancer therapies given in combination with gevokizumab are as follows:
Cohort A: Gevokizumab+FOLFOX+bevacizumab: Bevacizumab administered at 5 mg/kg IV on day 1 and 15 of a 28 day cycle. FOLFOX (also known as modified FOLFOX6): oxaliplatin administered at 85 mg/m2 IV, leucovorin (folinic acid) 400 mg/m2 IV, and bolus 5-fluorouracil 400 mg/m2 IV followed by 2400 mg/m2 as a 46-h continuous infusion on day 1 and 15 of a 28 day cycle.
Cohort B: Gevokizumab+FOLFIRI+bevacizumab: Bevacizumab administered at 5 mg/kg IV on day 1 and 15 of a 28 day cycle. FOLFIRI: irinotecan administered at 180 mg/m2 IV, leucovorin (folinic acid) 400 mg/m2 IV, and bolus 5-fluorouracil 400 mg/m2 IV followed by 2400 mg/m2 as a 46-h continuous infusion on day 1 and 15 of a 28 day cycle.
Cohort C: Gevokizumab+paclitaxel+ramucirumab: Ramucirumab administered at 8 mg/kg IV on day 1 and 15 of a 28 day cycle. Paclitaxel administered at 80 mg/m2 IV on days 1, 8, and 15 of a 28-day cycle.
Cohort D: Gevokizumab+cabozantinib: Cabozantinib administered at 60 mg orally once daily on a 28 day cycle.
The decision on dose tolerability for each cohort will be based on a review of safety data from the first 6 weeks (Cohorts A and B) or first 4 weeks (Cohorts C and D) of the combination treatment in Part 1b. For each cohort, the respective dose of the SOC anti-cancer therapies will be at the pre-determined dose level, and only dose levels of gevokizumab in the combination therapy will be assessed for safety review. A Bayesian logistic regression model for combinations using the escalation with overdose control criterion (EWOC) to evaluate the risk of dose-limiting toxicity (DLT) will guide the decision. Dose recommendations for each cohort will be based on summaries of the posterior distribution of DLT rate for each gevokizumab dose in combination with SOC anti-cancer therapies and will comply with the (EWOC) principle.

### Expansion Part (Part 2)

The objective of the expansion part is to assess the preliminary efficacy and safety of the combination therapy in each cohort. The progression-free survival (PFS) rate, assessed per RECIST v1.1 at specified landmark is the primary objective. PFS is defined as the time from the date of first dose of study treatment to the date of first documented radiological progression or death due to any cause. Overall response rate (ORR), disease control rate (DCR), duration of response (DOR) and overall survival (OS) are secondary objectives for all four cohorts; as well as assessing safety and tolerability of the combinations, and the immunogenicity and PK of gevokizumab in the combination regimens.
Part 2 will start when the RDE has been determined in part 1b (enrollment to each cohort will open independently of other cohorts):
Cohort A will enroll approximately 40 subjects with first line mCRC (20 subjects with hs-CRP≥10 mg/L+20 subjects with hs-CRP<10 mg/L). Subjects enrolled in Parts 1a/1b who were administered gevokizumab at the RDE will be included in the Part 2 subject numbers and analysis.
Cohort B will enroll approximately 40 subjects with second line mCRC (20 subjects with hs-CRP≥10 mg/L+20 subjects with hs-CRP<10 mg/L, all treated at the RDE of gevokizumab). Subjects enrolled in Parts 1a/1b that were administered gevokizumab at the RDE will be included in the Part 2 subject numbers and analysis.
Cohort C will enroll approximately 20 subjects with second line mGEC and hs-CRP≥10 mg/L. Subjects with hs-CRP≥10 mg/L who were enrolled in Part 1b and were administered gevokizumab at the RDE will be included in the Part 2 subject numbers and analysis.
Cohort D will enroll approximately 20 subjects with second/third line mRCC and hs-CRP≥10 mg/L. Subjects with hs-CRP≥10 mg/L who were enrolled in Part 1b and were administered gevokizumab at the RDE will be included in the Part 2 subject numbers and analysis.
   Patients will continue to receive the study treatment and be followed as per the schedule of assessments until disease progression per RECIST 1.1 or until discontinuation of the study for any reason. Approximately 172 subjects will be recruited in total in the study.
   PFS will be defined as the time from the date of first dose of study treatment to the date of first documented radiological progression or death due to any cause. Subjects will be analyzed by cohort, independently. Subjects in the safety run-in (Part 1b) treated at the RDE of gevokizumab in combination with the SOC anti-cancer therapies will be counted towards the number of subjects in the FAS of Part 2.
   Alternative to the cut-off value of hsCRP≥10 mg/L, patients having less inflammation status could also benefit from the treatment. In such cases cut-off value of hsCRP≥7 mg/L or cut-off value of hsCRP≥5 mg/L could be considered.

### Example 7

A Randomized, Open-Label, Phase II Study of Canakinumab or Pembrolizumab as Monotherapy and in Combination as Neoadjuvant Therapy in Subjects with Resectable Non-Small Cell Lung Cancer
The purpose of this randomized, open-label, phase II study is to evaluate the major pathological response (MPR) rate, a surrogate endpoint for overall survival (OS) and disease free survival (DFS) of canakinumab given as a neoadjuvant treatment, either as single agent or in combination with pembrolizumab, in addition to evaluate the MPR of pembrolizumab as a single agent and the dynamic of the tumor microenvironment changes on treatment by comparing pre-, on- and post-treatment samples.

| Objective(s) | Endpoint(s) |
|---|---|
| | Endpoint(s) for |
| Primary objective(s) | primary objective(s) |
| To assess the rate of MPR (≤10% of | Major pathologic |
| residual viable tumor cells) at | response (MPR) rate |
| the time of surgery in evaluable | (Assessed centrally) |
| subjects treated with canakinumab | |
| alone and in combination with | |
| pembrolizumab (central review) | |
| | Endpoint(s) for |
| | secondary |
| Secondary objective(s) | objective(s) |
| To assess surgical feasibility rate in | Surgical |
| each treatment arm based on | feasibility |
| randomized subjects | rate |
| To assess overall response rate in | Overall response rate |
| randomized subjects treated | based on local |
| with canakinumab or pembrolizumab | investigator |
| as monotherapy or in | assessment per |
| combination (local review) | RECIST 1.1 |
| To assess the rate of MPR at the | (a) MPR based on |
| time of surgery in (a) evaluable | central review |
| subjects in pembrolizumab | (b) MPR based on |
| monotherapy arm based on central | local review |
| review, (b) evaluable subjects | (c) MPR based on |
| based on local review in each | both central and local |
| treatment arm, (c) randomized | review |
| subjects based on both central and | (d) Difference in MPR |
| local review in each treatment | rate based on central |
| arm and (d) to estimate the | review |
| difference in MPR and posterior | |
| probability of the difference in | |
| MPR >= 10% between canakinumab + | |
| pembrolizumab combination | |
| and pembrolizumab alone | |
| based on central review | |

The study patients have confirmed stage IB-IIIA non-small cell lung cancer (NSCLC) planned for surgery in approximately 4-6 weeks.

### Inclusion Criteria

Histologically confirmed NSCLC stage IB-IIIA (per AJCC 8th edition), deemed suitable for primary resection by treating surgeon, except for N2 and T4 tumors.

### Exclusion Criteria

Subjects with unresectable or metastatic disease.
Subjects who received prior systemic therapy (including chemotherapy, other anti-cancer therapies and any other antibody or drug specifically targeting T-cell co-stimulation or immune checkpoint pathways) in the past 3 years before screening.
Subjects with brain metastasis are excluded from this study and all patients should have brain imaging (either MRI brain or CT brain with contrast) prior to enrollment.
   This is a phase II, randomized, open label study evaluating efficacy of canakinumab or pembrolizumab monotherapy or in combination as neoadjuvant treatment. Treatment arms include canakinumab alone or canakinumab in combination with pembrolizumab or pembrolizumab alone and receive two doses of canakinumab (200 mg s.c. Q3W) alone or in combination with pembrolizumab or pembrolizumab as single agent (200 mg i.v. Q3W)
   Subjects will be treated for a maximum duration of 6 weeks (2 cycles) until surgery, progression, unacceptable toxicity or discontinuation from the study treatment for any other reason. Surgery can be performed at anytime between 4 to 6 weeks after the first dose of study treatment. The primary endpoint is the major pathologic response (MPR) rate as assessed by the number of subjects with ≤10% residual viable cancer cells. Subjects will enter in the safety follow-up period up to 130 days after the last dose of study treatment.

### Example 8

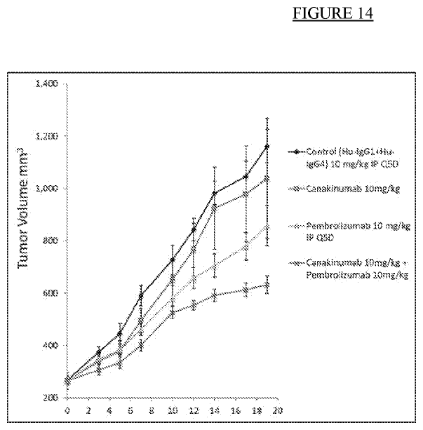

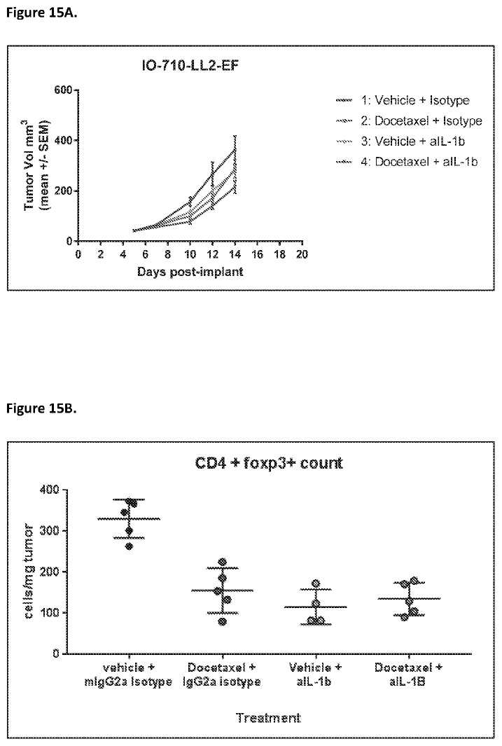

Preclinical Data on the Efficacy of Canakinumab in Combination with an Anti-PD-1 (Pembrolizumab) in the Treatment of Cancer.
A pilot study was designed to assess the impact of canakinumab as a monotherapy or in combination with anti-PD-1 (pembrolizumab) on tumor growth and the tumor microenvironment. A xenograft model of human NSCLC was created by subcutaneous injection of a human lung cancer cell line H358 (KRAS mutant) into BLT mouse xenograft model.
As shown in FIG. 14, the H358 (KRAS mutant) model is a very fast growing and aggressive model. In this model, combination treatment of canakinumab and pembrolizumab (shown in purple) led to a greater reduction than canakinumab single agent arm (shown in red) and pembrolizumab single agent treatment (shown in green), with a 50% decrease observed in the mean tumor volume when compared to the vehicle group.

### Example 9

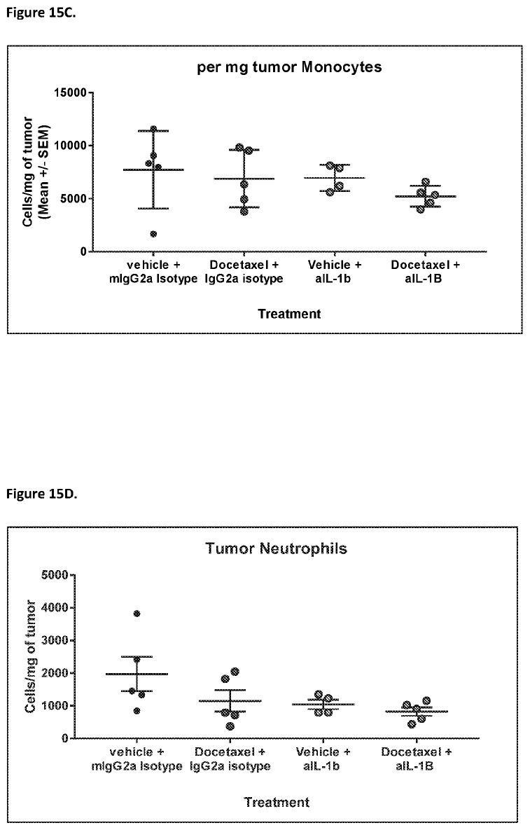

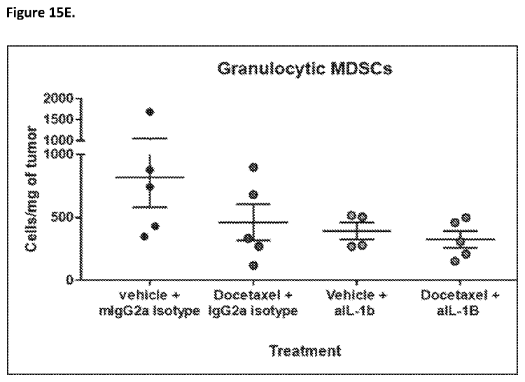

Preclinical Data on the Efficacy of Canakinumab in Combination with Docetaxel in the Treatment of Cancer.
In a study of anti-IL-1β in combination with docetaxel in an aggressive lung model (LL2), modest efficacy with anti-IL-1β was observed, as well as docetaxel alone. The efficacy was enhanced in the combination compared to either group alone or control (FIG. 15A). Decreases in immunosuppressive cells were observed with anti-IL-1β alone or in combination at the PD time point 5 days after the first dose, specifically in regulatory T cells and suppressive mouse myeloid cells including neutrophils, monocytes and MDSCs in tumors after IL-1β inhibition (FIG. 15B-E). These data support that the proposed mechanism of action in IL-1β inhibition can be demonstrated in vivo and also some efficacy of anti-IL-1β monotherapy was observed.

### Example 10

Treatment of 4T1 Tumors with 01BSUR and Docetaxel Leads to Alterations in the Tumor Microenvironment.
Female Balb/c mice with 4T1 tumors implanted subcutaneously (s.c.) on the right flank were treated 8 and 15 days post-tumor implant initiating when the tumors reached about 100 mm³ with the isotype antibody, docetaxel, 01BSUR, or a combination of docetaxel and 01BSUR. 01BSUR is the mouse surrogate antibody, since canakinumab does not cross-react to murine IL-1β. 01BSUR belongs to the mouse IgG2a subclass, which corresponds to human IgG1 subclass, which canakinumab belongs to. 5 days after the first dose, tumors were harvested and analyzed for changes to the infiltrating immune cell populations. This was done again at the end point of the study, 4 days after the second dose.

### Tumor Burden

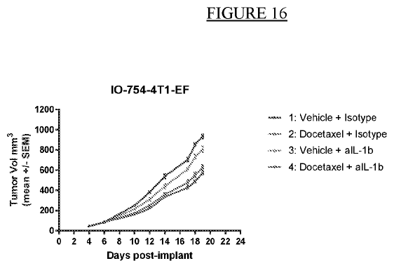

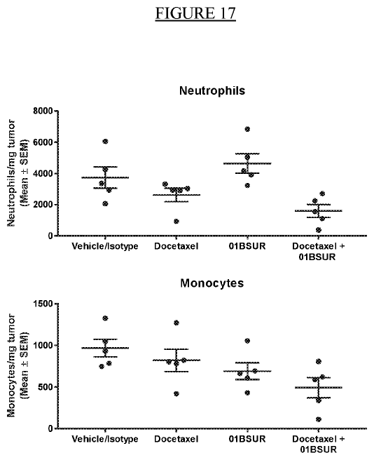

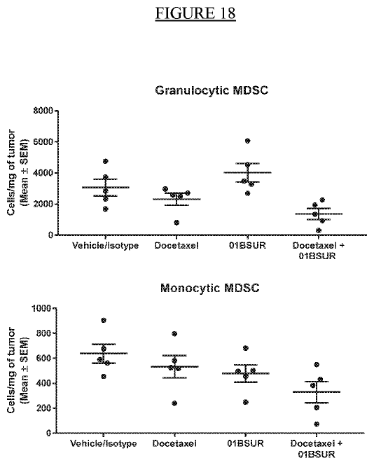

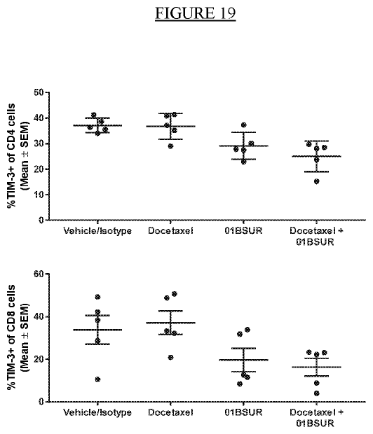

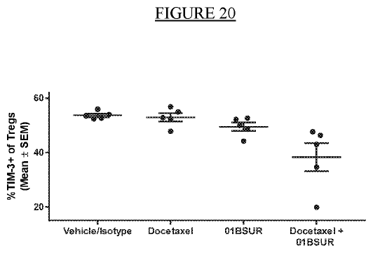

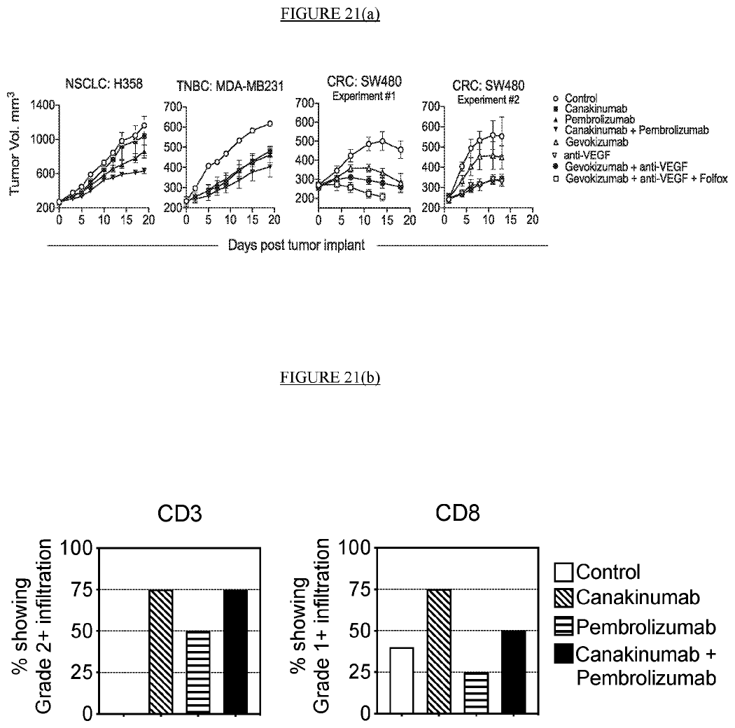

A slight slowing in tumor growth was seen in the 01BSUR anti-IL-1β alone treatment group compared to the vehicle/isotype control. This delay was enhanced in the single agent docetaxel group. The combination group showed a similar slowing in growth as the docetaxel alone group (FIG. 16).
TIL Analysis of 4T1 Tumors after a Single Dose of Docetaxel and 01BSUR—Myeloid Panel
Following a single treatment with docetaxel alone or in combination with 01BSUR, there was a decrease in neutrophils in the 4T1 tumors. The combination group, showed a greater decrease in neutrophil cell number than the docetaxel single agent group. Single agent 01BSUR led to a slight increase in neutrophils in 4T1 tumors, although this was not a significant change compared to the control group. Each of the treatments led to a decrease in monocytes compared to the vehicle/isotype group. The single agent 01BSUR treatment led to a greater decrease in monocytes than the docetaxel alone group. Further, the combination showed an even greater decrease in monocytes compared to the control group (P=0.0481) (FIG. 17). Similar trends to the granulocytes and monocytes were seen among the granulocytic and monocytic Myeloid derived suppressor cells (MDSC). Docetaxel alone and in combination with 01BSUR led to a decrease in granulocytic MDSC. All treatments led to a decrease in monocytic MDSC, with the combination leading to a greater decrease than either of the single agents (FIG. 18).
TIL Analysis of 4T1 Tumors after a Second Dose of Docetaxel and 01BSUR
Four days after a second dose of docetaxel and 01BSUR 4T1 tumors were analyzed for immune cell infiltrates. The percent of both CD4+ and CD8⁺ T cells expressing TIM-3 were determined. Docetaxel alone led to no change in the TIM-3 expressing cells compared to the control group, while there was a decrease in the TIM-3 expressing cells following treatment with 01BSUR alone or in combination with docetaxel. The combination group, appears to show a slightly larger decrease in TIM-3 expressing cells than the single agent 01BSUR group (P=0.0063) for CD4+ T cells compared to control (FIG. 19). Similar trends were seen in the Treg subset of cells with the combination group showing the largest level of decrease of the TIM-3 expressing cells (P=0.0064) compared to the control (FIG. 20).

### Conclusion and Discussion

Blocking IL-1β has been shown to be a potent method of changing the inflammatory microenvironment in autoimmune disease. ACZ885 (canakinumab) has been highly effective at treating some inflammatory autoimmune diseases, such as CAPS (Cryopyrin Associated Periodic Syndrome). As many tumors have an inflammatory microenvironment, blocking IL-1β is being studied to determine the impact that this will have on the tumor microenvironment alone and in combination with agents that will work to block the PD-1/PD-L1 axis or standard of care chemotherapeutic agents such as docetaxel. It has been shown through preclinical experiments and the CANTOS trial that the blockade of IL-1β can have an impact on tumor growth and development. However, the CANTOS trial, an atherosclerosis trial, evaluated this in a prophylactic setting with patients with no known or detectable cancer at the time of enrollment. Patients with established tumors or metastases may have different levels of response to IL-1β blockade.

These preliminary results studying combinations of 01BSUR, a murine surrogate of ACZ885, and docetaxel show that in the LL2 and 4T1 tumors models, this combination can have an impact on tumor growth.
The studies described here examine the TILs following a single treatment only (1D2 and 01BSUR combinations) or following two doses of each treatment (01BSUR and docetaxel). The overall trends alludes to a change in the suppressive nature of the TME in LL2 and 4T1 tumors.
While there is not a consistent change in the overall CD4+ and CD8⁺ T cells in the TME of these tumors, there is a trend towards in decrease in the Tregs in these tumors. Additionally, the Tregs typically also show a decrease in the percentage of cells expressing TIM-3. Tregs that express TIM-3 may be more effective suppressors of T cells than non-TIM-3 expressing Tregs [Sakuishi, 2013]. In several of the studies, there is an overall decrease of TIM-3 on all T cells. While the impact of this on these cells is not yet known, TIM-3 is a checkpoint and these cells may be more activated than the TIM-3 expressing T cells. However, further work is needed to understand these changes as some of the T cell changes observed could allude to a therapy that is less effective than the control.
While T cells make up a portion of the immune cell infiltrate in these tumors, a large portion of the infiltrating cells are myeloid cells. These cells were also analyzed for changes and IL-1β blockade consistently led to a decrease in the numbers of neutrophils and granulocytic MDSC in the tumors. Often these were accompanied by decreased monocytes and monocytic MDSC; however, there was more variability in these populations. Neutrophils both produce IL-1β and respond to IL-1β while MDSC generation is often dependent on IL-1β, and both subsets of cells can suppress the function of other immune cells. Decreases in both neutrophils and MDSC combined with a decrease in Tregs may mean that the tumor microenvironment becomes less immune suppressive following IL-1β blockade. A less suppressive TME may lead to a better anti-tumor immune response, particularly with checkpoint blockade.
These data taken together show that blocking both IL-1β and the PD-1/PD-L1 axis may lead to a more immune active tumor microenvironment or combining IL-1β blockade with chemotherapy may have a similar impact.

### Example 11

### Determining Immunogenicity/Allergenicity to IL-1β Antibody

During the CANTOS trial, blood samples for immunogenicity assessments were collected at baseline Month 12, 24 and end of study visit. Immunogenicity was analyzed using a bridging immunogenicity electrochemiluminescence immunoassay (ECLIA). Samples were pre-treated with acetic acid and neutralized in buffer containing labeled drug (biotinylated ACZ885 and sulfo-TAG (Ruthenium) labeled ACZ885). Anti-canakinumab antibodies (anti-drug antibodies) were captured by a combination of biotinylated and sulfo-TAG labeled forms of ACZ885. Complex formation was subsequently detected by electrochemiluminescence by capturing complexes on Mesoscale Discovery Streptavidin (MSD) plates.
Treatment-emergent anticanakinumab antibodies (anti-drug antibodies) were detected in low and comparable proportions of patients across all treatment groups (0.3%, 0.4% and 0.5% in the canakinumab 300 mg, 150 mg and placebo groups respectively) and were not associated with immunogenicity related AEs or altered hsCRP response.

### Example 12

Biomarker analysis from the CANTOS trial patients with gastroesophageal cancer, colorectal cancer and pancreatic cancer were grouped into GI group. Patients with bladder cancer, renal cell carcinoma and prostate cancer were grouped into GU group. Within the group, patients were further divided according to their baseline IL-6 or CRP level into above median group and below median group. The mean and median of time to cancer event were calculated as shown the table below.
There seems to have a trend that patient group have below median level of CRP and IL-6 had in general longer time to develop cancer. This trend seems to be stronger based on IL-6 analysis than CRP, possibly due to the fact that IL-6 is immediately downstream of IL-1β, where CTP is further away from IL-1β signaling and therefore could be influenced by other factors as well.

TABLE 6

| | | Time to cancer AE | | |
|---|---|---|---|---|
| Set | IL-6 median | N | Mean | Median |
| GI | Above median | 34 | 18.35 | 16.03 |
| | Below median | 35 | 27.84 | 28.55 |
| GU | Above median | 33 | 21.79 | 17.45 |
| | Below median | 33 | 27.85 | 23.39 |

TABLE 7

| | | Time to cancer AE | | |
|---|---|---|---|---|
| Set | CRP Median | N | Mean | Median |
| GI | Above median | 56 | 19.23 | 15.08 |
| | Below median | 58 | 25.61 | 26.17 |
| GU | Above median | 54 | 24.57 | 23.23 |
| | Below median | 56 | 25.15 | 24.13 |

### Examples 13 to 15

Examples 13 to 15 summarize canakinumab and gevokizumab pre-clinical work carried out in various cancer models.

### Materials and Methods:

I. Tumor models: The role of IL-1β in tumor immunity and efficacy of IL-1β blocking antibodies were tested in the following preclinical models:
   Xenograft tumors in humanized mouse models: NSCLC (H358), TNBC (MDA-MB231) and CRC (SW480)
   Syngeneic tumor models: TNBC (4T1) and lung (LL-2) cancer
II. IL-1β blockade and other combination treatments: blocking antibodies against human IL-1β (canakinumab and gevokizumab, both at 10 mg/Kg Q5D IP) and mouse IL-1β (clone 01BSUR, 10 mg/Kg Q5D IP) were tested in humanized xenograft and mouse syngeneic tumor models respectively. Combination treatments include chemotherapeutic agent, docetaxel (6.25 mg/Kg QW IV), PD-1 pathway inhibitors (anti-human PD-1, pembrolizumab 10 mg/Kg Q5D IP or anti-mouse PD-1, clone 1D2 10 mg/Kg QW IP) and anti-mouse VEGF blocking antibody (clone 4G3, 5 mg/Kg Q5D) were used in combination with anti-IL-1β antibody. Appropriate isotype controls were used. In all experiments, dosing was initiated after tumor implantation.
III. Experimental readouts: activity of therapeutic agents in preclinical models were assessed by the following:
   Tumor volume was determined by measurement with calipers throughout the course of the study. Tumor weights in milligrams were determined at end of study
   Immunohistochemistry analysis was performed on FFPE NSCLC (H358) xenograft tissue
   Changes in immune populations in peripheral blood in CRC (SW480) xenograft humanized model and tumors in syngeneic models (4T1 and LL-2) were assessed by flow cytometry using markers for T cell, myeloid populations

### Example 13

Results: Human IL-1β blocking antibodies, canakinumab and gevokizumab modulate tumor growth and immune responses in humanized BLT models

### NSCLC: H358 (Kras Mutant)

50% of animals in the canakinumab single agent arm show slower tumor growth compared to isotype control
100% of animals in the combination arm exhibit slower tumor growth compared to either single agent arms or isotype control
Canakinumab either alone and/or in combination with pembrolizumab has a more pronounced effect on CD8 and CD3 TIL infiltration

### TNBC: MDA-MB231

100% of animals in the canakinumab single agent arm has slower tumor growth compared to isotype control
Modest synergy seen in combination with Pembrolizumab

### CRC: SW480

In one of two experiments, significant reduction in tumor volume seen with gevokizumab in 100% of animals. In the second experiment, 80% of animals in the gevokizumab arm had slower tumor growth compared to isotype control
Tumor volume reduction seen in the combination arm with anti-VEGF is driven by anti-VEGF
Increase in CD45+ immune cells observed in gevokizumab+anti-VEGF combination arm
An increase in CD68+ myeloid cells and a decrease in tolerogenic DC-10 immune populations are observed following combination blockade of IL-1β and VEGF
   Canakinumab even as a single agent has a pronounced effect on the numbers of CD8 and CD3+ TILs infiltrating the NSCLC tumor compared to single agent pembrolizumab and isotype control. The combination of pembrolizumab and canakinumab maintains the levels seen with single agent canakinumab. Despite comparable levels to single agent canakinumab, It is possible that blocking PD-1 removes certain brakes on the effector T cells that get recruited to the TME following canakinumab treatment and this might result in better tumor growth control compared to canakinumab alone. As such, the type of CD8 effector responses might be qualitatively distinct from that seen in the single agent arm, as we see greater effect in tumor growth in the combination arm.

### Example 14

Results: IL-1β blockade remodels the TME and slows tumor growth in combination with docetaxel in syngeneic mouse models

### IL-1β Blockade as a Single Agent:

IL-1β inhibition results in decreased infiltration of neutrophils, TAMs, granulocytic, and monocytic MDSCs
IL-1β blockade also results in improved CD8/Treg ratio (increased CD8 effector T cells and decreased FoxP3+ Tregs

### Anti-IL-1β/Docetaxel Combination:

Tumor growth reduction seen in LL-2 model with docetaxel/aIL-1β combo
Evidence for TME remodeling in Docetaxel/IL-1β combination

### Anti-IL-1β/Anti-PD-1 Combination (Data not Shown):

Immunomodulatory effect seen with anti-IL-1β are not observed with anti-PD-1 alone and is maintained in combination with anti-PD-1

### Example 15

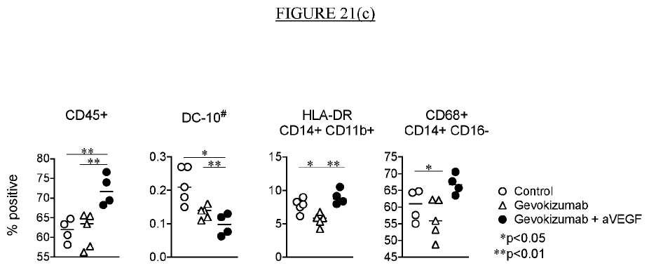

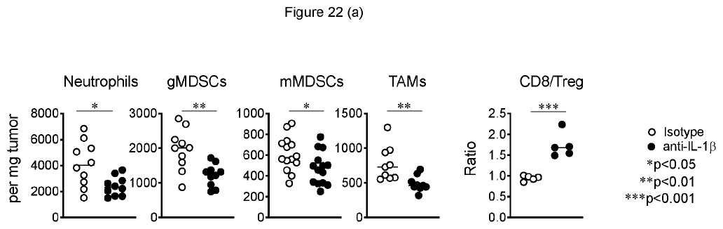

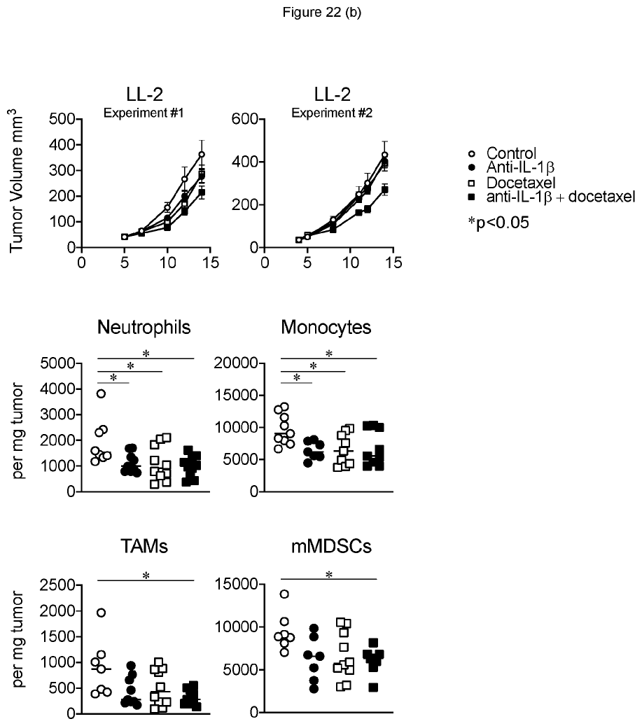

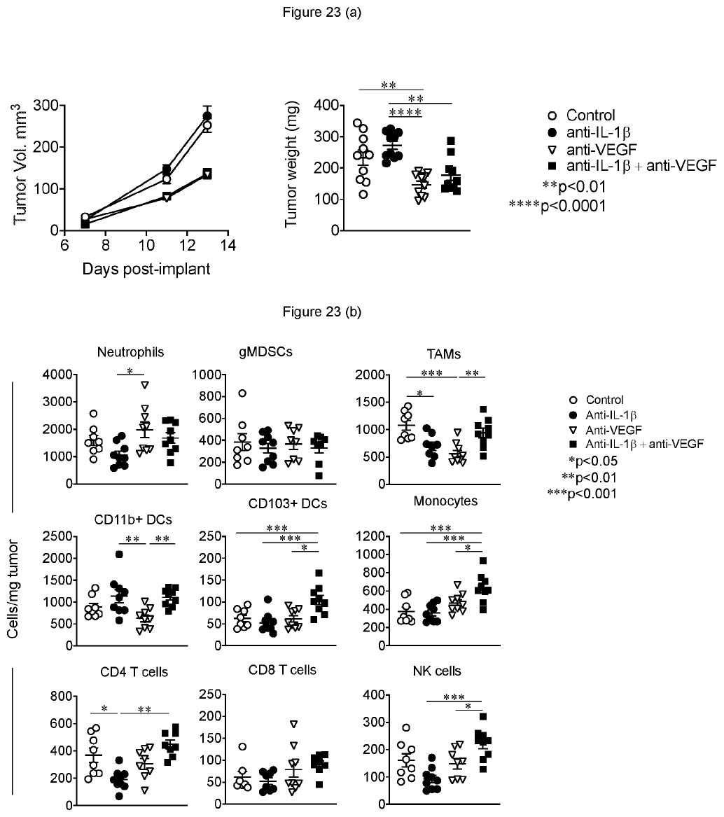

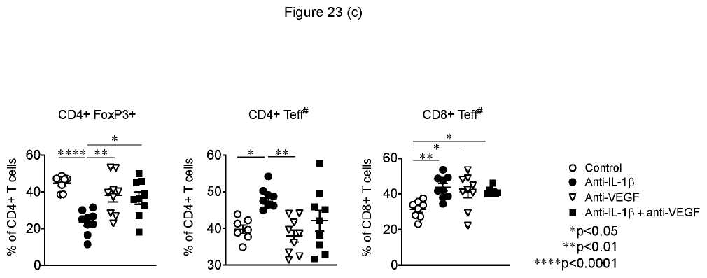

Results: IL-1β/VEGF combination blockade exhibits altered TME in 4T1 syngeneic model
Similar to SW480/BLT model (FIG. 21a), tumor growth reduction in the combination arm of anti-IL-1β and anti-VEGF in 4T1 syngeneic model is driven by VEGF blockade. Even though we do not see reduction in tumor volume and weight with single agent anti-IL-1β antibody, we see ample evidence for modulation of TME, such as reduction in immune suppressive cells (neutrophils, TAMs and FoxP3+ Tregs) as seen in FIGS. 21b and 21c. We see further modulation in the combination arm where addition of anti-IL-1β augments numbers of protective immune cells such as CD103+ DCs, and NK cells compared to either single agent alone strongly indicating of synergy in modulating the TME.
The syngeneic models that are being used are particularly aggressive and within the duration of study, it is hard to see tumor control. What we do see is immune modulation following perturbation of the IL-1β/VEGF pathways.
Regulation of multiple immune subsets, some of which are driven by IL-1β or VEGF single agent arms, while others are unique to the combination arm
Single agent IL-1β blockade results in decrease in PMNs, TAMs, and FoxP3+ Tregs
Single agent VEGF blockade increases PMNs and decreases in CD11b+ DCs and TAMs
IL-1/VEGF combination blockade result in a synergistic increase in mMDSCs, CD103+ DCs, monocytes and NK cells; while recalibrating the CD4 loss seen with single agent IL-1β
   Conclusion of Pre-Clinical Results from Examples 13 to 15
Canakinumab demonstrates increased CD8+ and CD3+ T-cells and preliminary antitumor activity as a monotherapy and in combination with anti-PD-1 using humanized mouse models of lung cancer and TNBC.
Gevokizumab exhibits significant anti-tumor activity as monotherapy in humanized mouse models of CRC, with modulation of peripheral myeloid cells in gevokizumab and anti-VEGF combination arms
IL-1β inhibition in syngeneic models results in immunomodulation including a decrease in immunosuppressive cells including Tregs, neutrophils, monocytes and MDSCs
Greater efficacy and TME immunomodulation are observed in models with the combination of anti-VEGF+anti-IL-1β, docetaxel+anti-IL-1β and anti-PD-1+anti-IL-1β as compared to either treatment alone

### Example 16

### Clinical Confirmation of Canakinumab Dose

A Randomized, Double-Blind Phase III Study of Pembrolizumab+Platinum-based Chemotherapy with or without Canakinumab as First Line Therapy for Locally Advanced or Metastatic Non-squamous and Squamous Non-small Cell Lung Cancer Subjects
The study population includes adult patients with first-line locally advanced stage IIIB (not eligible for definitive chemo-radiation therapy) or stage IV metastatic non-small cell lung cancer (NSCLC), without EGFR mutations or ALK translocation. Only patients who have not previously been treated with any systemic anti-cancer therapy are included, with the exception of neo-adjuvant or adjuvant therapy (if relapse has occurred more than 12 months from the end of that therapy). In addition, subjects should be without known B-RAF mutation or ROS-1 genetic aberrations.

### Safety Run-In Prior to Start of Phase III Study

The non-randomized safety run-in portion of the study will be done with canakinumab in combination with pembrolizumab and three platinum-based doublet chemotherapies: carboplatin+pemetrexed (patients with non-squamous tumors), cisplatin+pemetrexed (patients with non-squamous tumors), and carboplatin+paclitaxel (patients with either squamous or non-squamous tumor). Non-squamous tumor histology subjects who receive paclitaxel-carboplatin with pembrolizumab in the safety-run-in and achieve stable disease (SD) or better will receive pemetrexed maintenance after completing induction. The canakinumab dose will start at 200 mg every three weeks (Q3W).
The primary objective is to determine the recommended Phase III dose regimen (RP3R) of canakinumab in combination with pembrolizumab and chemotherapy. The secondary objectives are to characterize safety and tolerability, pharmacokinetics, immunogenicity, and to assess the preliminary clinical anti-tumor activity.
The analysis to determine the recommended phase III dose regimen (RP3R) will be conducted when at least 6 evaluable patients in each of the 3 treatment cohorts have been observed for dose limiting toxicity (DLT) for at least 42 days at the starting dose level to establish the RP3R. Evaluable patients will be defined as follows:
Having received the full dose pembrolizumab 200 mg IV for at least 2 cycles (21 day=1 cycle) and at least 75% of the planned dose of 2 cycles of chemotherapy, and
Having received at least 2 doses of canakinumab at 200 mg s.c. either every 3 weeks or every 6 weeks, and
Been followed for at least 42 days for adverse events.

### Results

In the safety run in part of the study patients are divided to 3 cohorts.
Cohort A (non-squamous): canakinumab+pembrolizumab+carboplatin+pemetrexed
Cohort B (non-squamous): canakinumab+pembrolizumab+cisplatin+pemetrexed
Cohort C (squamous or non-squamous): canakinumab+pembrolizumab+carboplatin+paclitaxel
   30 patients (10 in cohort A [A], 11 in cohort B [B], and 9 in cohort C [C]) were treated. At data cut-off, 6 patients (3 in A, 2 in B and 1 in C) of the 30 treated patients, discontinued the study treatment. The primary reason for treatment discontinuation was PD (3 patients in A and 1 patient each in B and C)

### Dose-Limiting Toxicity and Recommended Phase 3 Dose Regimen (RP3R)

Overall, only 1 patient experienced one DLT during the first 42 days of study treatment (cohort C: grade 3 hepatitis, deemed related to pembrolizumab as per investigator assessment)
Based on BLRM and all relevant data, the RP3R of canakinumab as 200 mg SC Q3W combined with pembrolizumab and platinum doublet based chemotherapy was supported

### Safety

Overall, 83% of the patients received 3 doses or more of study treatment (50% of patients received 3 doses, 30% of patients 4 doses and 3% of patients 5 doses). In cohort A in particular, 7 of 10 patients received 4 doses of study treatment at time of data cut-off.
1 patient died due to study indication
AEs leading to dose reduction of any of the study medications across all cohorts were reported only in cohort C, namely myalgia (1 of 9 patients) and peripheral neuropathy (2 of 9 patients) both leading to chemotherapy reduction
AEs leading to dose interruption across all cohorts were decreased neutrophil count (3 [10%] patients); decreased white blood cell count and neutropenia (1 [3.3%] patient each)
AEs leading to discontinuation of one of the study drugs were reported in total in 3 (10%) patients from cohort C (hepatitis leading to pembrolizumab discontinuation, peripheral neuropathy and polyneuropathy leading to chemotherapy discontinuation) but none were considered to be related to canakinumab
No Grade 5 adverse events were observed
Overall, 13 patients (43.3%) experienced grade 3 AEs and 1 patient experienced grade 4 AE
   The RP3R of canakinumab in combination with standard dose of pembrolizumab and Ctx was 200 mg SC Q3W.

### Example 17

### IL-1β Neutralization Sensitises Pancreatic Tumors to Anti-PD-1 Checkpoint Therapy

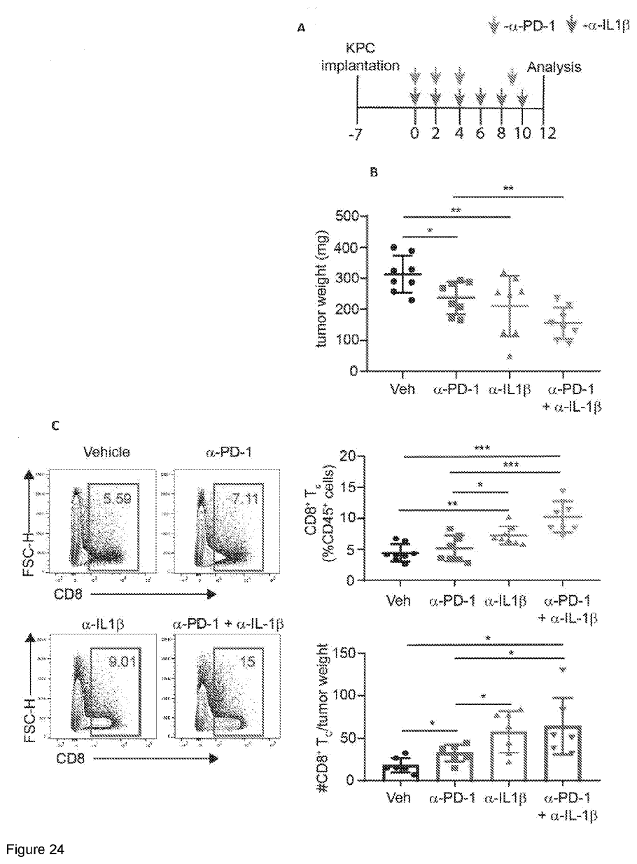

5×10⁴ KPC cells (Sunil R. Hingorani et al, Cancer Cell, 2005, 469-483) were orthotopically injected into the pancreas of C57BL/6 mice (David Tuveson. On day 7 post-injection, mice were intraperitoneally administered either 10 mg/kg anti-mouse PD-1, 10 mg/kg anti-mouse IL-1β (01BSUR) or IgG control antibody diluted in 200 μL of sterile PBS. While anti-PD-1 antibody was administered at days 7, 9, and 11 and 16 post injection of KPC cells, anti-IL-1β was administered every 2 days post KPC implantation.
The poor response of pancreatic tumors to immune checkpoint blockade has been primarily attributed to its immunosuppressive microenvironment and poor CD8⁺ T cell infiltration (Johnson B A 3rd, etc. Clin Cancer Res 2017; 23:1656-1669.). Since depletion of tumor-derived IL-1β significantly increases CD8⁺ T cell infiltration and activity, we reasoned that IL-1 neutralization could sensitize PDA tumors to PD-1 checkpoint blockade. To this end, KPC tumor bearing mice were treated with neutralising antibodies against IL-1β and PD-1 (FIG. 24A). Indeed, addition of α-IL-1p treatment significantly enhanced the anti-tumor activity of α-PD-1 (FIG. 24B). As predicted, combined treatment of α-IL-1β and α-PD-1 resulted in increased tumor infiltration of CD8⁺ T cells, relative to vehicle control or α-PD-1 alone (FIG. 24 C).

### FIG. 24. IL-1β Neutralization Sensitises PDA Tumors to PD-1 Checkpoint Blockade.

A. Schematic of anti-IL-IP and anti-PD-1 antibody treatment regimen. Treatment was initiated one week post orthotopic implantation of KPC cells. Green arrows indicate anti-PD-1 antibody administration while red arrows correspond to anti-IL-1β antibody treatment. B. Graph represents quantification of analysis in A, indicating tumor weight (N=8). Error bars indicate SD; P-values determined by the Student t test (two-tailed, unpaired). Data representative of 2 independent experiments. C. Representative flow cytometry plots (left) of KPC tumors treated with vehicle control, anti-PD-1 antibody alone, anti-IL-1β antibody alone or bothanti-PD-1 and anti-IL-1β antibody, indicating tumor infiltrating CD8⁺ T cells. Graphs depict quantitation of FACS analysis, represented as either percentage of CD45⁺ immune cells (top right, N=8) or absolute number of CD8⁺ T cells relative to tumor weight (bottom right, N=7). Error bars indicate SD; P-values determined by the Student t test (two-tailed, unpaired). Data representative of 2 independent experiments. *p<0.05; **p<0.01; ***p<0.001; ****p<0.0001.

1. An IL-1β binding antibody or a functional fragment thereof for use in a patient in the treatment and/or prevention of a cancer, wherein an therapeutically effective amount of IL-1β binding antibody or a functional fragment thereof is administered to the patient every 3 weeks or every 4 weeks for at least 13 months.

2. An IL-1β binding antibody or a functional fragment thereof for use in a patient in the treatment of a cancer, wherein the hazard ratio of cancer mortality of the patient is reduced by at least 10%.

3. An IL-1β binding antibody or a functional fragment thereof for use in a patient in the treatment of a cancer, wherein the patient has at least 3 months progression free survival (PFS).

4. An IL-1β binding antibody or a functional fragment thereof for use in a patient in the treatment of a cancer, wherein the PFS of the patient is at least 3 months progression free survival (PFS) longer than the standard of care treatment of the cancer.

5. An IL-1β binding antibody or a functional fragment thereof for use in a patient in the treatment of a cancer, wherein the patient has at least 3 months overall survival (OS).

6. An IL-1β binding antibody or a functional fragment thereof for use in a patient in the treatment of a cancer, wherein the patient has at least 3 months overall survival (OS) longer than standard of care treatment.

7. An IL-1β binding antibody or a functional fragment thereof for use in a patient in the treatment of a cancer, wherein the patient is not at high risk of developing serious infection.

8. An IL-1β binding antibody or a functional fragment thereof for use in a patient in the treatment of a cancer, wherein the IL-1β binding antibody or a functional fragment thereof is not administered in combination with a TNF inhibitor.

9. An IL-1β binding antibody or a functional fragment thereof for use in a patient in the treatment of a cancer, wherein the patient has at least 3 months disease free survival (DFS).

10. An IL-1β binding antibody or a functional fragment thereof for use in a patient in the treatment of a cancer, wherein the IL-1β binding antibody or a functional fragment thereof is canakinumab, wherein the chance of the patient developing anti-canakinumab antibody is less than 1%.

11. An IL-1β binding antibody or a functional fragment thereof for use in a patient in the treatment of a cancer, wherein a therapeutically effective amount of IL-1β binding antibody or a functional fragment thereof is administered to the patient by an auto-injector.

12. The use according to any one of the preceding claims, wherein the cancer is selected from a list consisting of lung cancer, especially NSCLC, colorectal cancer (CRC), melanoma, gastric cancer (including esophageal cancer), renal cell carcinoma (RCC), breast cancer, prostate cancer, head and neck cancer (including oral), bladder cancer, hepatocellular carcinoma (HCC), ovarian cancer, cervical cancer, endometrial cancer, pancreatic cancer, neuroendocrine cancer, hematological cancer (particularly multiple myeloma, acute myeloblastic leukemia (AML)), and biliary tract cancer.

13. The use according to any one of the preceding claims, wherein said cancer is not lung cancer or NSCLC.

14. The use according to any one of the preceding claims, wherein said cancer is a cancer having at least partial inflammatory basis.

15. The use according to any one of the preceding claims, where the IL-1β binding antibody or a functional fragment thereof is canakinumab.

16. The use according to claim 15, wherein the therapeutic effective amount of canakinumab is about 200 mg.

17. The use according to claim 16, wherein canakinumab is administered every 3 weeks or every 4 weeks.

18. The use according to any one of the claims 15 to 17, wherein canakinumab is administered is subcutaneously.

19. The use according to any one of the claims 1-14, where the IL-1β binding antibody or a functional fragment thereof is gevokizumab.

20. The use according to claim 19, wherein the therapeutic effective amount of gevokizumab is about 30-120 mg.

21. The use according to claim 20, wherein gevokizumab is administered every 3 weeks or every 4 weeks.

22. The use according to any one the claims 19 to 21, wherein gevokizumab is administered intravenously or subcutaneously.

23. The use according to any one of the preceding claims, wherein said cancer is colorectal cancer (CRC).

24. The use according to any one of the claims 1-22, wherein said cancer is renal cell carcinoma (RCC).

25. The use according to any one of the claims 1-22, wherein said cancer is breast cancer, preferably TNBC.

26. The use according to any one of the claims 1-22, wherein said cancer is gastric cancer.

27. The use according to any one of the claims 1-22, wherein said cancer is melanoma.

28. The use according to any one of the claims 1-22, wherein said cancer is pancreatic cancer.

29. The use according to any one of the claims 1-22, wherein said cancer is prostate cancer.

30. The use according to any one of the claims 1-22, wherein said cancer is bladder cancer.

31. The use according to any of the preceding claims, wherein said patient has high sensitivity C-reactive protein (hsCRP) equal to or greater than about 3.2 mg/L before first administration of said IL-1β binding antibody or functional fragment thereof.

32. The use according to any one of the preceding claims, wherein said IL-1β binding antibody or a functional fragment thereof is administered in combination with one or more therapeutic agents.

33. The use according to claim 32, wherein the one or more therapeutic agents is the standard of care agent for the cancer.

34. The use according to claim 32 or 33, wherein the one or more therapeutic agents is a check point inhibitor.

35. The use according to claim 34, wherein the check point inhibitor is selected from a list consisting of nivolumab, pembrolizumab, atezolizumab, durvalumab, avelumab, Ipilimumab and spartalizumab.

36. The use according to claim 34, wherein the check point inhibitor is pembrolizumab.

37. The use according to any one of the preceding claims, wherein said IL-1β binding antibody or a functional fragment thereof is used, alone or preferably in combination, in the prevention of recurrence or relapse of cancer having at least a partial inflammatory basis in a subject after said cancer has been surgically removed.

38. The use according to any one of the preceding claims, wherein said IL-1β binding antibody or a functional fragment thereof is used, alone or preferably in combination, as the first, second or third line treatment.

39. The use according to any one of the preceding claims, wherein said IL-1β binding antibody or a functional fragment thereof is used, alone or preferably in combination, as the first, second or third line treatment.

40. The use according to any one of the preceding claims, wherein said IL-1β binding antibody or a functional fragment thereof is used, alone or preferably in combination, for more than one lines of treatment in the same patient.
